(19) United States
(12) Patent Application Publication
(10) Pub. No.: US 2012/0129833 A1
(43) Pub. Date: May 24, 2012

(54) CYCLIC KETOENOLS FOR THERAPY

(75) Inventors: Ningshu LIU, Berlin (DE); Kai Thede, Berlin (DE); Philip Lienau, Berlin (DE); Arne Scholz, Berlin (DE); Maher Najjar, Berlin (DE); Ulf Bomer, Glienicke/Nordbahn (DE); Knut Eis, Berlin (DE); Reiner Fischer, Monheim am Rhein (DE)

(73) Assignee: BAYER PHARMA AKTIENGESELLSCHAFT, Berlin (DE)

(21) Appl. No.: 13/213,436

(22) Filed: Aug. 19, 2011

Related U.S. Application Data
(60) Provisional application No. 61/375,375, filed on Aug. 20, 2010.

(51) Int. Cl. A61K 31/553; A61K 31/407; A61P 3/04; A61P 3/10; A61P 3/06; A61P 1/16; A61P 9/00; C07D 209/54; A61K 31/403; C07D 207/38; A61K 31/4015; C07D 491/113; C07D 307/94; A61K 31/365; C07D 493/10; C07D 487/04; A61K 31/5025; C07D 498/04; A61K 31/4162; C07D 231/34; A61K 31/4152; C07D 221/20; A61K 31/438; C07D 491/107

(52) U.S. Cl. 514/211.08; 548/410; 514/409; 548/408; 548/543; 514/424; 548/551; 549/265; 514/462; 514/451; 544/235; 514/248; 540/545; 548/360.1; 514/405; 548/366.4; 514/404; 546/16; 514/278

(57) ABSTRACT

The invention relates to 5′-biphenyl-substituted cyclic ketoenols for therapeutic purposes, to pharmaceutical compositions and to their use in therapy, in particular for the prophylaxis and therapy of diabetes, obesity, hyperlipidaemia, fatty livers and of disorders of the cardiovascular system.

This application claims the benefit of the filing date of U.S. Provisional Application Ser. No. 61/375,375 filed Aug. 20, 2010, which is incorporated by reference herein.

The present invention relates to 5′-biphenyl-substituted cyclic ketoenols for therapeutic purposes, to pharmaceutical compositions and to their use in therapy, in particular for the prophylaxis and therapy of diabetes, obesity, hyperlipidaemia, fatty livers and of disorders of the cardiovascular system.

Acetyl-CoA carboxylases (ACCs) play a key role in cellular fatty acid homeostasis. ACCs are biotin-containing enzymes which catalyze the carboxylation of acetyl-CoA to malonyl-CoA in an ATP-dependent manner (Kim, 1997; Harwood, 2005; Tong, 2005). This reaction, which proceeds as two semi-reactions, a biotin carboxylase (BC) reaction and a carboxyltransferase (CT) reaction, is the first initial step in the fatty acid biosynthesis and is the rate-determining step of the pathway. Two human ACC isoforms, ACC1 and ACC2, are known, which are encoded by different genes (LuTFI ABU-ELHEIGA, et al, 1995, Jane WIDMER, et al. 1996). ACC1 is expressed in lipogenic tissue (liver, fatty tissue), is localized in the cytosol and fills the malonyl-CoA pool which serves as C2 unit donor for the de novo synthesis of long-chain fatty acids by FASN and subsequent chain elongation. ACC2 is expressed in particular in oxidative tissues (liver, heart, skeletal muscle) (Bianchi et al., 1990; Kim, 1997), is associated with the mitochondria, and regulates a second pool of malonyl-CoA. This regulates the fatty acid oxidation by inhibiting carnitinipalmitoyl transferase I, the enzyme which facilitates the import of long-chain fatty acids into the mitochondria for β-oxidation (Milgraum L Z, et al., 1997, Widmer J. et al., 1996). Both enzymes have very high sequence homology and are regulated in a similar manner by a combination of transcriptional, translational and prosttranslational mechanisms. In humans as well as in animals, the ACC activity is under the strict control of a number of dietary, hormonal and other physiological mechanisms which act through forward allosteric activation by citrate, feedback inhibition by long-chain fatty acids, reversible phosphorylation and inactivation and modulation of the enzyme production by modified gene expression.

ACC1 knockout mice are embryonally lethal (Swinnen, et al., 2006, Abu-Elheiga, et al. 2005). ACC2 knockout mice show reduced malonyl-CoA concentrations in skeletal and heart muscle, increased fatty acid oxidation in the muscle, reduced liver fat levels, reduced amounts of total body fat, increased levels of UCP3 in skeletal muscle (as a sign of increased energy output), reduced body weight, lower plasma concentrations of free fatty acids, reduced plasma glucose levels, reduced amounts of tissue glycogen, and they are protected against diet-induced diabetes and obesity (Abu-Elheiga et al., 2001, 2003; Oh et al., 2005).

In addition to being involved in the fatty acid synthesis in lipogenic tissues and the fatty acid oxidation in oxidative tissues, an upregulation of ACC and an increased lipogenesis was observed in many tumour cells (Swinnen, et al., 2004, Heemers, et al., 2000, Swinnen, et al., 2002, Rossi, et al., 2003, Milgraum, et al., 1997, Yahagi, et al., 2005). With high probability, this phenotype contributes in the development and progression of tumours; however, the associated regulatory mechanisms still have to be elucidated.

A number of substances capable of inhibiting insect-ACC have been found.

PCT patent application PCT/EPP99/01787, published as WO 99/48869, which corresponds to the European patent EP 1 066 258 B1, relates to novel arylphenyl-substituted cyclic ketoenols, to a plurality of processes for their preparation and to their use as pesticides and herbicides.

Pharmaceutical properties of 3-acylpyrrolidine-2,4-diones have been described in the prior art (S. Suzuki et al. Chem. Pharm. Bull. 15 1120 (1967)). Furthermore, N-phenylpyrrolidine-2,4-diones have been synthesized by R. Schmierer and H. Mildenberger (Liebigs Ann. Chem. 1985, 1095). A biological activity of these compounds has not been described.

EP-A-0 262 399 and GB-A-2 266 888 disclose compounds of a similar structure (3-arylpyrrolidine-2,4-diones); however, these compounds have not been known to have any herbicidal, insecticidal or acaricidal activity. Known to have herbicidal, insecticidal or acaricidal activity are unsubstituted bicyclic 3-arylpyrrolidine-2,4-dione derivatives (EP-A-355 599, EP-A-415 211 and JP-A-12-053 670), and also substituted monocyclic 3-arylpyrrolidine-2,4-dione derivatives (EP-A-377 893 and EP-A-442 077).

Also known are polycyclic 3-arylpyrrolidine-2,4-dione derivatives (EP-A-442 073) and also 1H-arylpyrrolidinedione derivatives (EP-A-456 063, EP-A-521 334, EP-A-596 298, EP-A-613 884, EP-A-613 885, WO 95/01971, WO 95/26 954, WO 95/20 572, EP-A-0 668 267, WO 96/25 395, WO 96/35 664, WO 97/01 535, WO 97/02 243, WO 97/36 868, WO 97/43275, WO 98/05638, WO 98/06721, WO 98/25928, WO 99/24437, WO 99/43649, WO 99/48869, WO 99/55673, WO 01/17972, WO 01/23354, WO 01/74770, WO 03/013249, WO 03/062244, WO 2004/007448, WO 2004/024 688, WO 04/065366, WO 04/080962, WO 04/111042, WO 05/044791, WO 05/044796, WO 05/048710, WO 05/049569, WO 05/066125, WO 05/092897, WO 06/000355, WO 06/029799, WO 06/056281, WO 06/056282, WO 06/089633, WO 07/048,545, DEA 102 00505 9892, WO 07/073,856, WO 07/096,058, WO 07/121,868, WO 07/140,881, WO 08/067,873, WO 08/067,910, WO 08/067,911, WO 08/138,551, WO 09/015,801, WO 09/039,975, WO 09/049,851, PCT/EP2008/005973, PCT/EP2008/007517, EP application numbers 08153002 and 08170489. Moreover, ketal-substituted 1-H-arylpyrrolidine-2,4-diones are known from WO 99/16748 and (spiro)-ketal-substituted N-alkoxyalkoxy-substituted arylpyrrolidinediones are known from JP-A-14 205 984 and Ito M. et al. Bioscience, Biotechnology and Biochemistry 67, 1230-1238, (2003). The addition of safeners to ketoenols is likewise known in principle from WO 03/013249. Moreover, WO 06/024411 discloses herbicidal compositions which comprise ketoenols.

It is known that certain substituted Δ³-dihydrofuran-2-one derivatives have herbicidal properties (cf. DE-A-4 014 420). The synthesis of the tetronic acid derivatives used as starting materials (such as, for example, 3-(2-methylphenyl)-4-hydroxy-5-(4-fluorophenyl)-Δ³-dihydrofuranone-(2)) is likewise described in DE-A-4 014 420. Compounds of a similar structure are known from the publication Campbell et al., J. Chem. Soc., Perkin Trans. 1, 1985, (8) 1567-76, without any insecticidal and/or acaricidal activity being stated. 3-Aryl-Δ³-dihydrofuranone derivatives having herbicidal, acaricidal and insecticidal properties are furthermore known from: EP-A-528 156, EP-A-647 637, WO 95/26 954, WO 96/20 196, WO 96/25 395, WO 96/35 664, WO 97/01 535, WO 97/02 243, WO 97/36 868, WO 98/05 638, WO 98/06 721, WO 99/16 748, WO 98/25 928, WO 99/43 649, WO 99/48 869, WO 99/55 673, WO 01/23354, WO 01/74 770, WO 01/17 972, WO 04/024 688, WO 04/080 962, WO 04/111 042, WO 05/092 897, WO 06/000 355, WO 06/029 799, WO 07/048,545, WO 07/073,856, WO 07/096,058, WO 07/121,868, WO 07/140,881, WO 08/067,911, WO 08/083,950, PCT/EP2008/005973, PCT/EP/2008/007517.

3-Aryl-Δ³-dihydrothiophenone derivatives are known from WO 95/26 345, 96/25 395, WO 97/01 535, WO 97/02 243, WO 97/36 868, WO 98/05638, WO 98/25928, WO 99/16748, WO 99/43649, WO 99/48869, WO 99/55673, WO 01/17972, WO 01/23354, WO 01/74770, WO 03/013249, WO 04/080 962, WO 04/111 042, WO 05/092897, WO 06/029799 and WO 07/096,058.

Certain phenylpyrone derivatives which are unsubstituted in the phenyl ring are already known (cf. A. M. Chirazi, T. Kappe and E. Ziegler, Arch. Pharm. 309, 558 (1976) and K.-H. Boltze and K. Heidenbluth, Chem. Ber. 91, 2849). Phenylpyrone derivatives which are substituted in the phenyl ring and have herbicidal, acaricidal and insecticidal properties are described in EP-A-588 137, WO 96/25 395, WO 96/35 664, WO 97/01 535, WO 97/02 243, WO 97/16 436, WO 97/19 941, WO 97/36 868, WO 98/05638, WO 99/43649, WO 99/48869, WO 99/55673, WO 01/17972, WO 01/74770, WO 03/013249, WO 04/080 962, WO 04/111 042, WO 05/092897, WO 06/029799 and WO 07/096,058.

Certain 5-phenyl-1,3-thiazine derivatives which are unsubstituted in the phenyl ring are already known (cf. E. Ziegler and E. Steiner, Monatsh. 95, 147 (1964), R. Ketcham, T. Kappe and E. Ziegler, J. Heterocycl. Chem. 10, 223 (1973)). 5-Phenyl-1,3-thiazine derivatives which are substituted in the phenyl ring and have herbicidal, acaricidal and insecticidal properties are described in WO 94/14 785, WO 96/25 395, WO 96/35 664, WO 97/01 535, WO 97/02 243, WO 97/02 243, WO 97/36 868, WO 99/43649, WO 99/48869, WO 99/55673, WO 01/17972, WO 01/74770, WO 03/013249, WO 04/080 962, WO 04/111 042, WO 05/092897, WO 06/029799 and WO 07/096,058.

It is known that certain substituted 2-arylcyclopentanediones have herbicidal, insecticidal and acaricidal properties (cf., for example, U.S. Pat. Nos. 4,283,348; 4,338,122; 4,436,666; 4,526,723; 4,551,547; 4,632,698; WO 96/01 798; WO 96/03 366, WO 97/14 667 and also WO 98/39281, WO 99/43649, WO99/48869, WO 99/55673, WO 01/17972, WO 01/74770, WO 03/062244, WO 04/080962, WO04/111042, WO05/092897, WO06/029799, WO07/080,066, WO07/096,058 and EP application number 08166352). Compounds having similar substitutions are also known; 3-hydroxy-5,5-di-methyl-2-phenylcyclopent-2-en-1-one from the publication Micklefield et al., Tetrahedron, (1992), 7519-26 and also the natural compound involutin (−)-cis-5-(3,4-dihydroxyphenyl)-3,4-dihydroxy-2-(4-hydroxyphenyl)cyclopent-2-enone from the publication Edwards et al., J. Chem. Soc. S, (1967), 405-9. An insecticidal or acaricidal action is not described. Moreover, 2-(2,4,6-trimethylphenyl)-1,3-indanedione is known from the publication J. Economic Entomology, 66, (1973), 584 and the laid-open application DE-A 2 361 084, with herbicidal and acaricidal activities being stated.

It is known that certain substituted 2-arylcyclohexanediones have herbicidal, insecticidal and acaricidal properties (U.S. Pat. Nos. 4,175,135, 4,256,657, 4,256,658, 4,256,659, 4,257,858, 4,283,348, 4,303,669, 4,351,666, 4,409,153, 4,436,666, 4,526,723, 4,613,617, 4,659,372, DE-A 2 813 341, and also Wheeler, T. N., J. Org. Chem. 44, 4906 (1979)), WO 99/43649, WO 99/48869, WO 99/55673, WO 01/17972, WO 01/74770, WO 03/013249, WO 04/080 962, WO 04/111 042, WO 05/092897, WO 06/029799, WO 07/096,058, WO 08/071,405, WO 08/110,307, WO 08/110,308, WO 09/074,314 and WO 08/145,336).

It is known that certain substituted 4-arylpyrazolidine-3,5-diones have acaricidal, insecticidal and herbicidal properties (cf., for example, WO 92/16 510, EP-A-508 126, WO 96/11 574, WO 96/21 652, WO 99/47525, WO 01/17 351, WO 01/17 352, WO 01/17 353, WO 01/17 972, WO 01/17 973, WO 03/028 466, WO 03/062 244, WO 04/080 962, WO 04/111 042, WO 05/005428, WO 05/016873, WO 05/092897, WO 06/029799 and WO 07/096,058).

It is known that certain tetrahydropyridones have herbicidal properties (JP 0832530). Specific 4-hydroxytetrahydropyridones having acaricidal, insecticidal and herbicidal properties are also known (JP 11152273). Furthermore, 4-hydroxytetrahydropyridones have been disclosed as pesticides and herbicides in WO 01/79204 and WO 07/096,058. 4-Hydroxyquinolones are disclosed in WO 03/01045.

It is known that certain 5,6-dihydropyrone derivatives as protease inhibitors have antiviral properties (WO 95/14012). Furthermore, 4-phenyl-6-(2-phenethyl)-5,6-dihydropyrone is known from the synthesis of kawalactone derivatives (Kappe et al., Arch. Pharm. 309, 558-564 (1976)). Moreover, 5,6-dihydropyrone derivatives are known as intermediates (White, J. D., Brenner, J. B., Deinsdale, M. J., J. Amer. Chem. Soc. 93, 281-282 (1971)). 3-Phenyl-5,6-dihydropyrone derivatives with applications in crop protection are described in WO 01/98288 and WO 07/09658.

4-Biphenyl-substituted tetronic acid derivatives for the therapy of viral disorders are disclosed in WO 2008/022725.

WO 2005/089118 and WO2007/039286 disclose, in a general manner, nitrogenous bicyclic structures for therapy, 5′-biphenyl-substituted cyclic ketoenols not being specifically mentioned.

4-Phenyl-substituted [1.2]-oxazine-3,5-diones as herbicides were initially described in WO 01/17972. Furthermore, 4-acyl-substituted [1.2]-oxazine-3,5-diones as pesticides, but especially as herbicides and growth regulators, are described, for example, in EP-A-39 48 89; WO 92/07837, U.S. Pat. No. 5,728,831, and as herbicides and pesticides in WO 03/048138.

Based on this prior art, it was an object of the present invention to provide novel structures for the therapy of disorders. The structures according to the invention should be suitable in particular for the prophylaxis and therapy of diabetes, obesity, hyperlipidaemia, fatty livers and of disorders of the cardiovascular system and have advantages compared to the structures known from the prior art.

Surprisingly, it has now been found that a specific sub-group of the aryl-substituted cyclic ketoenols described in the prior art also inhibits human ACC and is suitable for the therapy of disorders.

Here, it was unforeseeable whether and which of the structures known as insecticides or herbicides would achieve the object of the invention, that is to say to provide structures which can be used in the therapy of human disorders.

Applicant is unaware of any 5′-biphenyl-substituted cyclic ketoenols in accordance with the present invention being described in the prior art for the therapy of disorders, in particular not for the therapy of diabetes, obesity, hyperlipidaemia, fatty livers and of disorders of the cardiovascular system.

The object is achieved by compounds of the formula (I).

It has now been found that compounds of the formula (I)

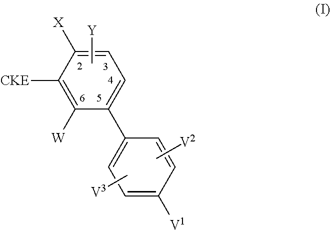

in which
X represents halogen, nitro or cyano or represents an optionally monohalogen- or polyhalogen-substituted C₁-C₆-alkyl, C₁-C₆-alkoxy, C₁-C₆-alkoxy-C₁-C₆-alkoxy, C₃-C₇-cycloalkyl or a C₃-C₇-cycloalkyl-C₁-C₆-alkoxy radical, and
W and Y independently of one another represent hydrogen, nitro, cyano or halogen or represent an optionally monohalogen- or polyhalogen-substituted C₁-C₆-alkyl, C₁-C₆-alkoxy or C₃-C₇-cycloalkyl radical, and
V¹, V² and V³ independently of one another represent hydrogen, halogen, nitro or cyano or represent a C₁-C₆-alkyl, halo-C₁-C₆-alkyl, C₁-C₆-alkoxy, halo-C₁-C₆-alkoxy, C₁-C₆-alkylthio, C₁-C₆-alkylsulphinyl, C₁-C₆-alkylsulphonyl, C₁-C₆-alkoxy-C₁-C₆-alkyl, C₃-C₁₀-cycloalkyl radical or represent a monocyclic heterocycloalkyl radical, and/or
V¹ and V² together with the carbon atoms to which they are attached form a saturated or unsaturated cycle T¹ which optionally contains at least one further heteroatom and has 4 to 7 ring atoms and whose ring-forming atoms may be mono- or polysubstituted by identical or different substituents selected from the group consisting of halogen and C₁-C₆-alkyl radicals,
CKE represents one of the groups

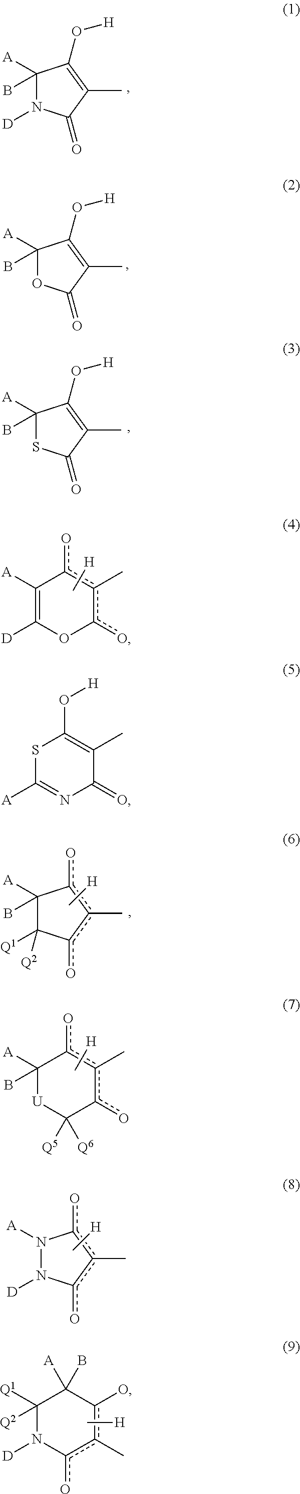

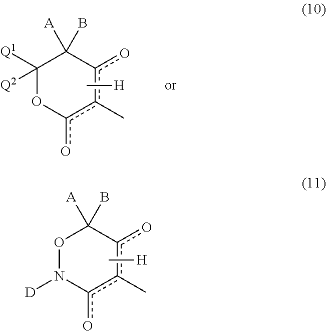

in which
U represents —S—, —S(O)—, —S(O)₂—, —O—,

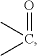

a substituted

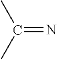

group
   or represents a C₁-C₄-alkylene group which is optionally substituted by Q³ and Q⁴, and
A represents hydrogen or
   represents an optionally monohalogen- or polyhalogen-substituted
   C₁-C₆-alkyl, C₂-C₆-alkenyl, C₁-C₆-alkoxy-C₁-C₆-alkyl or
   C₁-C₆-alkylthio-C₁-C₆-alkyl radical or
   represents a C₃-C₇-cycloalkyl or monocyclic heterocyclyl radical, each of which may be mono- or polysubstituted by identical or different substituents selected from the group consisting of halogen and C₁-C₆-alkyl radicals or
   represents an aryl, aryl-C₁-C₆-alkyl or heteroaryl radical, each of which may optionally be mono- or polysubstituted by identical or different substituents selected from the group consisting of halogen, cyano, nitro and C₁-C₆-alkyl, halo-C₁-C₆-alkyl, C₁-C₆-alkoxy and halo-C₁-C₆-alkoxy radicals and
B represents hydrogen or represents a C₁-C₆-alkyl or C₁-C₆-alkoxy-C₁-C₆-alkyl radical, or
A and B together with the carbon atom to which they are attached form a saturated or unsaturated cycle T² which optionally contains at least one heteroatom and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen, hydroxyl or cyano or
   b) represent C₁-C₆-alkyl, C₁-C₆-alkoxy, C₁-C₆-alkoxy-C₁-C₆-alkyl, C₁-C₆-alkylcarbonyl,
      C₁-C₆-alkoxycarbonyl, C₁-C₆-alkylaminocarbonyl, C₁-C₆-alkylthio, C₁-C₆-alkylsulphinyl, C₁-C₆-alkylsulphonyl, C₁-C₆-alkylaminosulphonyl, C₁-C₆-alkoxy-C₁-C₆-alkoxy, halo-C₁-C₆-alkyl or halo-C₁-C₆-alkoxy radical which is optionally hydroxyl-substituted in the alkyl moiety, or
   c) represent an aryl, arylcarbonyl, arylsulphonyl, arylamino, heteroaryl, heteroarylcarbonyl, heteroarylsulphonyl or heteroarylamino radical, or
   d) represent a C₃-C₇-cycloalkyl, C₃-C₇-cycloalkylcarbonyl, C₃-C₇-cycloalkylsulphonyl, heterocyclyl, heterocyclylcarbonyl or heterocyclylsulphonyl radical,
      where the radicals mentioned under c) and d) may optionally be mono- or polysubstituted at the ring system by identical or different substituents selected from the group consisting of halogen, hydroxyl, cyano, nitro and halo-C₁-C₆-alkoxy, C₁-C₆-alkoxy-C₁-C₆-alkyl, C₃-C₁₀-cycloalkyl and 3- to 6-membered heterocycloalkyl radicals, and/or
   e) two of the radicals R¹, R² and R³ together with the ring atom(s) of the cycle T² to which they are attached may form a further saturated or unsaturated cycle T³ which optionally contains at least one heteroatom and has 3 to 7 ring atoms and may be mono- or polysubstituted by identical or different substituents selected from the group consisting of the radicals R⁴, R⁵ and R⁶,
      where R⁴, R⁵ and R⁶ independently of one another represent a C₁-C₆-alkyl or C₁-C₆-alkoxy radical, and
D represents hydrogen or
   represents a C₁-C₆-alkyl, C₂-C₆-alkenyl, C₂-C₆-alkynyl or C₁-C₆-alkoxy-C₁-C₆-alkyl radical or
   represents a C₃-C₇-cycloalkyl or monocyclic heterocyclyl radical or
   represents an aryl, aryl-C₁-C₆-alkyl, heteroaryl or heteroaryl-C₁-C₆-alkyl radical, where the radicals mentioned may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl, C₃-C₁₀-cycloalkyl and monocyclic heterocycloalkyl radicals, or
A and D together with the atoms to which they are attached form a saturated or unsaturated cycle T⁴ which optionally contains at least one further heteroatom and has 3 to 7 ring atoms, which may be bridged and whose ring-forming atoms may be mono- or polysubstituted by identical or different substituents selected from the group consisting of the radicals R⁷, R⁸ and R⁹,
   where R⁷, R⁸ and R⁹ independently of one another represent hydroxyl, halogen or represent a C₁-C₆-alkyl or C₁-C₆-alkoxy radical, and
A and Q¹ together with the atoms to which they are attached form a saturated or unsaturated cycle T⁵ which optionally contains at least one further heteroatom and has 5 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals,
   with the proviso that B and Q² represent a bond if the cycle T⁵ formed by A and Q¹ is aromatic,
Q¹ represents hydrogen or
   represents a C₁-C₆-alkyl or C₁-C₆-alkoxy radical which is optionally mono- or polysubstituted by identical or different substituents selected from the group consisting of halogen, hydroxyl and a C₁-C₆-alkoxy radical or
   represents a C₃-C₇-cycloalkyl or monocyclic heterocyclyl radical, each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals or
   represents a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, and
Q², Q⁴, Q⁵ and Q⁶ independently of one another represent hydrogen or represent a C₁-C₆-alkyl radical, and
Q³ represents hydrogen or
   represents a C₁-C₆-alkyl or C₁-C₆-alkoxy radical which is optionally mono- or polysubstituted by identical or different substituents selected from the group consisting of halogen, hydroxyl and a C₁-C₆-alkoxy radical or
   represents a C₃-C₇-cycloalkyl or monocyclic heterocyclyl radical, each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals or
   represents a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, or
Q¹ and Q² together with the carbon atom to which they are attached form a saturated or unsaturated and cycle T⁶ which optionally contains at least one further heteroatom having 3 to 7 ring atoms,
   whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, or
Q³ and Q⁴ together with the carbon atom to which they are attached form a saturated or unsaturated cycle T⁷ which optionally contains at least one heteroatom and has 3 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals,
   are suitable for use as a medicament.

The medicaments are suitable for the prophylaxis and therapy of human or animal disorders, in particular for the prophylaxis and therapy of diabetes, obesity, hyperlipidaemia, fatty livers and of disorders of the cardiovascular system.

Accordingly, the present invention provides 5′-biphenyl-substituted ketoenols of the formula (I) for therapeutic purposes, pharmaceutical compositions and their use in therapy, in particular for the prophylaxis and therapy of diabetes, obesity, hyperlipidaemia, fatty livers and of disorders of the cardiovascular system.

The invention is based on the following definitions:

### Alkyl:

Alkyl represents a straight-chain or branched saturated monovalent hydrocarbon radical having generally 1 to 6 (C₁-C₆-alkyl), preferably 1 to 4 (C₁-C₄-alkyl), and particularly preferably 1 to 3 carbon atoms (C₁-C₃-alkyl).

The following may be mentioned by way of example and by way of preference:

methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl.

Particular preference is given to a methyl, ethyl, propyl or isopropyl radical.

### Alkylene=Alkanediyl:

Alkylene represents a straight-chain or branched saturated divalent hydrocarbon radical having generally 1 to 6 (C₁-C₆-alkylene), preferably 1 to 4 (C₁-C₄-alkylene), and particularly preferably 1 to 3 (C₁-C₃-alkylene) carbon atoms.

The following may be mentioned by way of example and by way of preference:

methylene, ethylene, propylene, butylene, pentylene, hexylene, isopropylene, isobutylene, sec-butylene, tert-butylene, isopentylene, 2-methylbutylene, 1-methylbutylene, 1-ethylpropylene, 1,2-dimethylpropylene, neo-pentylene, 1,1-dimethylpropylene, 4-methylpentylene, 3-methylpentylene, 2-methylpentylene, 1-methylpentylene, 2-ethylbutylene, 1-ethylbutylene, 3,3-dimethylbutylene, 2,2-dimethylbutylene, 1,1-dimethylbutylene, 2,3-dimethylbutylene, 1,3-dimethylbutylene, 1,2-dimethylbutylene.

Particular preference is given to methylene, ethylene or propylene.

### Alkenyl:

Alkenyl represents a straight-chain or branched monovalent hydrocarbon radical having at least one double bond and generally 2 to 6 (C₂-C₆-alkenyl), preferably 2 to 4 (C₂-C₄-alkenyl), and particularly preferably 2 or 3 (C₂-C₃-alkenyl) carbon atoms.

The following may be mentioned by way of example and by way of preference:

vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methyl-pent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, methylhexadienyl.

Particular preference is given to vinyl or allyl.

### Alkenylene=Alkenediyl:

Alkenylene represents a straight-chain or branched divalent hydrocarbon radical having at least one double bond and generally 2 to 6 (C₂-C₆-alkenylene), preferably 2 to 4 (C₂-C₄-alkenylene), and particularly preferably 2 or 3 (C₂-C₃-alkenylene) carbon atoms.

### Alkynyl:

Alkynyl represents a straight-chain or branched monovalent hydrocarbon radical having at least one triple bond and generally 2 to 6 (C₂-C₆-alkynyl), preferably 2 to 4 (C₂-C₄-alkynyl), and particularly preferably 2 or 3 (C₂-C₃-alkynyl) carbon atoms.

The following may be mentioned by way of example and by way of preference:

ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl.

Particular preference is given to ethynyl, prop-1-ynyl or prop-2-ynyl.

### Cycloalkyl:

Cycloalkyl represents a mono- or bicyclic saturated monovalent hydrocarbon radical having generally 3 to 10 (C₃-C₁₀-cycloalkyl), preferably 3 to 8 (C₃-C₈-cycloalkyl), and particularly preferably 3 to 7 (C₃-C₇-cycloalkyl) carbon atoms.

The following may be mentioned by way of example and by way of preference for monocyclic cycloalkyl radicals:

cyclopropyl, cyclobutyl, cyclopentyl, cyclohexyl and cycloheptyl.

Particular preference is given to a cyclopropyl, a cyclopentyl or a cyclohexyl radical.

The following may be mentioned by way of example for bicyclic cycloalkyl radicals:

perhydropentalenyl, decalinyl.

### Cycloalkylcarbonyl

Cycloalkylcarbonyl represents the group —C(O)-cycloalkyl.

### Cycloalkylsulphonyl

Cycloalkylsulphonyl represents the group —S(O)₂-cycloalkyl.

### Cycloalkylalkoxy:

Cycloalkylalkoxy represents an alkoxy radical which is substituted by a cyclic saturated hydrocarbon ring.

Here, Cₙ-cycloalkyl-Cₘ-alkoxy means that the alkoxy moiety has m carbon atoms and the cycloalkyl moiety has n carbon atoms.

The following may be mentioned by way of example and by way of preference:

cyclopropylmethyl, cyclobutylethyl, cyclopentylethyl.

### Alkoxy:

Alkoxy represents a straight-chain or branched saturated alkyl ether radical of the formula —O-alkyl having generally 1 to 6 (C₁-C₆-alkoxy), preferably 1 to 4 (C₁-C₄-alkoxy), and particularly preferably 1 to 3 (C₁-C₃-alkoxy) carbon atoms.

The following may be mentioned by way of example and by way of preference:

methoxy, ethoxy, n-propoxy, isopropoxy, tert-butoxy, n-pentoxy and n-hexoxy.

### Alkylthio

Alkylthio represents a straight-chain or branched saturated alkylthio ether radical of the formula —S-alkyl having generally 1 to 6 (C₁-C₆-alkylthio), preferably 1 to 4 (C₁-C₄-alkylthio), and particularly preferably 1 to 3 (C₁-C₃-alkylthio) carbon atoms.

### Alkoxyalkyl

Alkoxyalkyl represents an alkyl radical substituted by alkoxy.

Here, Cₙ-alkoxy-Cₘ-alkyl means that the alkoxy moiety has n carbon atoms and the alkyl moiety through which the radical is attached has m carbon atoms.

### Alkylthioalkyl

Alkylthioalkyl represents an alkyl radical substituted by alkylthio.

Here, Cₙ-alkylthio-Cₘ-alkyl means that the alkylthio moiety has n carbon atoms and the alkyl moiety through which the radical is attached has m carbon atoms.

### Alkoxyalkoxy

Alkoxyalkoxy represents an alkoxy radical substituted by alkoxy.

Here, Cₙ-alkoxy-Cₘ-alkoxy means that the outer alkoxy moiety has n carbon atoms and the alkoxy moiety through whose oxygen function the radical is attached has m carbon atoms.

### Alkylamino

Alkylamino represents an amino radical having one or two alkyl substituents (selected independently of one another) having generally 1 to 6, preferably 1 to 3, carbon atoms.

(C₁-C₃)-Alkylamino, for example, represents a monoalkylamino radical having 1 to 3 carbon atoms or represents a dialkylamino radical having in each case 1 to 3 carbon atoms per alkyl substituent.

The following may be mentioned by way of example:

methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

### Alkylcarbonyl

Alkylcarbonyl represents the group —C(O)-alkyl having generally 1 to 6, preferably 1 to 4, and particularly preferably 1 to 3 carbon atoms in the alkyl moiety.

The following may be mentioned by way of example:

acetyl and propanoyl.

### Alkoxycarbonyl

Alkoxycarbonyl represents the group —C(O)—O-alkyl having generally 1 to 6, preferably 1 to 4, and particularly preferably 1 to 3 carbon atoms in the alkyl moiety.

The following may be mentioned by way of example:

methoxycarbonyl, ethoxycarbonyl, propoxycarbonyl, isopropoxycarbonyl, tert-butoxycarbonyl, n-pentoxycarbonyl and n-hexoxycarbonyl.

### Alkylaminocarbonyl

Alkylaminocarbonyl represents the group —C(O)-alkylamino having one or two alkyl substituents (selected independently of one another) having generally 1 to 6, preferably 1 to 3, carbon atoms.

(C₁-C₃)-Alkylaminocarbonyl, for example, represents a monoalkylaminocarbonyl radical having 1 to 3 carbon atoms or represents a dialkylaminocarbonyl radical having in each case 1 to 3 carbon atoms per alkyl substituent.

The following may be mentioned by way of example:

methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino carbonyl and N-n-hexyl-N-methylaminocarbonyl.

### Alkylsulphinyl

Alkylsulphinyl represents a straight-chain or branched saturated radical of the formula —S(O)-alkyl having generally 1 to 6 (C₁-C₆-alkylsulphinyl), preferably 1 to 4 (C₁-C₄-alkylsulphinyl), and particularly preferably 1 to 3 (C₁-C₃-alkylsulphinyl) carbon atoms.

The following may be mentioned by way of example and by way of preference:

methylsulphinyl, ethylsulphinyl, propylsulphinyl.

### Alkylsulphonyl

Alkylsulphonyl represents a straight-chain or branched saturated radical of the formula —S(O)₂-alkyl having generally 1 to 6 (C₁-C₆-alkylsulphonyl), preferably 1 to 4 (C₁-C₄-alkylsulphonyl), and particularly preferably 1 to 3 (C₁-C₃-alkylsulphonyl) carbon atoms.

The following may be mentioned by way of example and by way of preference:

methylsulphonyl, ethylsulphonyl, propylsulphonyl.

### Alkylaminosulphonyl

Alkylaminosulphonyl represents the group —S(O)₂-alkylamino having one or two alkyl substituents (selected independently of one another) having generally 1 to 6, preferably 1 to 3, carbon atoms.

(C₁-C₃)-Alkylaminosulphonyl, for example, represents a monoalkylaminosulphonyl radical having 1 to 3 carbon atoms or represents a dialkylaminosulphonyl radical having in each case 1 to 3 carbon atoms per alkyl substituent.

The following may be mentioned by way of example:

methylaminosulphonyl, ethylaminosulphonyl, n-propylaminosulphonyl, isopropylaminosulphonyl, tert-butylaminosulphonyl, n-pentylaminosulphonyl, n-hexylaminosulphonyl, N,N-dimethyl-aminosulphonyl, N,N-diethylaminosulphonyl, N-ethyl-N-methylaminosulphonyl, N-methyl-N-n-propylaminosulphonyl, N-isopropyl-N-n-propylaminosulphonyl, N-t-butyl-N-methylaminosulphonyl, N-ethyl-N-n-pentylamino-sulphonyl and N-n-hexyl-N-methylaminosulphonyl.

### Aryl

Aryl is a monovalent aromatic mono- or bicyclic ring system without any heteroatoms having 6 or 10 carbon atoms.

The following may be mentioned by way of example and by way of preference:

phenyl (C₆-aryl), naphthyl (C₁₀-aryl).

Particular preference is given to phenyl.

### Arylcarbonyl

Arylcarbonyl represents the group —C(O)-aryl.

### Arylsulphonyl

Arylsulphonyl represents the group —S(O)₂-aryl.

### Arylamino

Arylamino represents the group —NR-aryl, where R represents hydrogen or alkyl.

### Arylalkyl

Arylalkyl represents a straight-chain or branched saturated monovalent alkyl group substituted by an aromatic mono- or bicyclic ring system without any heteroatoms.

Here, Cₙ-aryl-Cₘ-alkyl means that the alkyl moiety has m carbon atoms and the aryl moiety has n carbon atoms.

The following may be mentioned by way of example and by way of preference:

phenylmethyl, phenylethyl.

### Heteroatoms

Heteroatoms are to be understood as meaning oxygen, nitrogen or sulphur atoms.

### Heteroaryl

Heteroaryl is a monovalent mono- or bicyclic ring system having at least one heteroatom and at least one aromatic ring. The heteroatoms present may be nitrogen atoms, oxygen atoms and/or sulphur atoms. The binding valency may be at any aromatic carbon atom or at a nitrogen atom.

A monocyclic heteroaryl radical in accordance with the present invention has 5 or 6 ring atoms.

Heteroaryl radicals having 5 ring atoms include, for example, the rings:

thienyl, thiazolyl, furyl, pyrrolyl, oxazolyl, imidazolyl, pyrazolyl, isoxazolyl, isothiazolyl, oxadiazolyl, triazolyl, tetrazolyl and thiadiazolyl.

Heteroaryl radicals having 6 ring atoms include, for example, the rings:

pyridyl, pyridazinyl, pyrimidinyl, pyrazinyl and triazinyl.

A bicyclic heteroaryl radical in accordance with the present invention has 9 or 10 ring atoms.

Heteroaryl radicals having 9 ring atoms include, for example, the rings:

phthalidyl, thiophthalidyl, indolyl, isoindolyl, indazolyl, benzothiazolyl, benzofuryl, benzothienyl, benzimidazolyl, benzoxazolyl, azocinyl, indolizinyl, purinyl, indolinyl.

Heteroaryl radicals having 10 ring atoms include, for example, the rings:

isoquinolinyl, quinolinyl, quinolizinyl, quinazolinyl, quinoxalinyl, cinnolinyl, phthalazinyl, 1,7- or 1,8-naphthyridinyl, pteridinyl, chromanyl.

Monocyclic heteroaryl rings having 5 or 6 ring atoms are preferred.

### Heteroarylcarbonyl

Heteroarylcarbonyl represents the group —C(O)-heteroaryl.

### Heteroarylsulphonyl

Heteroarylsulphonyl represents the group —S(O)₂-heteroaryl.

### Heteroarylamino

Heteroarylamino represents the group —NR-heteroaryl, where R represents hydrogen or alkyl.

### Heteroarylalkyl

Heteroarylalkyl represents a straight-chain or branched saturated monovalent alkyl group substituted by an aromatic mono- or bicyclic ring system having at least one heteroatom different from carbon.

Here, monocyclic heteroaryl-Cₘ-alkyl means that the alkyl moiety has m carbon atoms and the heteroaryl moiety is monocyclic and therefore has 5 or 6 ring atoms.

### Heterocyclyl

Heterocyclyl for the purpose of the invention is a non-aromatic mono- or bicyclic ring system having at least one heteroatom or a hetero group. The heteroatoms present may be nitrogen atoms, oxygen atoms and/or sulphur atoms. The hetero groups present may be —S(O)—, —S(O)₂— or —N⁺(O⁻)—. A monocyclic heterocyclyl ring in accordance with the present invention may have 3 to 8, preferably 5 to 8, particularly preferably 5 or 6, ring atoms.

The following may be mentioned by way of example and by way of preference for monocyclic heterocyclyl radicals having 3 ring atoms:

aziridinyl.

The following may be mentioned by way of example and by way of preference for monocyclic heterocyclyl radicals having 4 ring atoms:

azetidinyl, oxetanyl.

The following may be mentioned by way of example and by way of preference for monocyclic heterocyclyl radicals having 5 ring atoms:

pyrrolidinyl, imidazolidinyl, pyrazolidinyl, pyrrolinyl, dioxolanyl and tetrahydrofuranyl.

The following may be mentioned by way of example and by way of preference for monocyclic heterocyclyl radicals having 6 ring atoms:

piperidinyl, piperazinyl, morpholinyl, dioxanyl, tetrahydropyranyl and thiomorpholinyl.

The following may be mentioned by way of example and by way of preference for monocyclic heterocyclyl radicals having 7 ring atoms:

azepanyl, oxepanyl, [1,3]-diazepanyl, [1,4]-diazepanyl.

The following may be mentioned by way of example and by way of preference for monocyclic heterocyclyl radicals having 8 ring atoms:

oxocanyl, azocanyl.

A bicyclic heterocyclyl radical in accordance with the present invention may have 5 to 12, preferably 8 to 10, ring atoms.

Preference is given to 5- to 8-membered monocyclic saturated heterocyclyl radicals having up to two heteroatoms from the group consisting of O, N and S.

Particular preference is given to morpholinyl, piperidinyl and pyrrolidinyl.

### Heterocyclylcarbonyl

Heterocyclylcarbonyl represents the group —C(O)-heterocyclyl.

### Heterocyclylsulphonyl

Heterocyclylsulphonyl represents the group —S(O)₂-heterocyclyl.

### Halogen

The term halogen includes fluorine, chlorine, bromine and iodine.

Preference is given to fluorine and chlorine.

Haloalkyl:

Haloalkyl represents an alkyl radical having at least one halogen substituent.

The following may be mentioned by way of example and by way of preference:

trifluoromethyl, 2,2,2-trifluoroethyl, pentafluoroethyl, 5,5,5,4,4-pentafluoropentyl or 5,5,5,4,4,3,3-heptafluoropentyl.

Preference is given to perfluorinated alkyl radicals such as trifluoromethyl or pentafluoroethyl.

### Haloalkoxy

Haloalkoxy represents an alkoxy radical having at least one halogen substituent.

Preference is given to fluoroalkoxy radicals.

The following may be mentioned by way of example and by way of preference:

trifluoromethoxy or 2,2,2-trifluoroethoxy.

### Cycle

Cycle includes all ring systems.

### Unsaturated Cycle

An unsaturated cycle includes ring systems having at least one double bond in the ring and aromatic ring systems.

In formula (I), X may represent:

halogen, nitro or cyano or
an optionally monohalogen- or polyhalogen-substituted C₁-C₆-alkyl, C₁-C₆-alkoxy, C₁-C₆-alkoxy-C₁-C₆-alkoxy, C₃-C₇-cycloalkyl or a C₃-C₇-cycloalkyl-C₁-C₆-alkoxy radical.

In formula (I), X may preferably represent:

halogen or an optionally monohalogen- or polyhalogen-substituted C₁-C₃-alkyl or C₁-C₃-alkoxy radical.

In formula (I), W and Y independently of one another may represent:

hydrogen, nitro, cyano or halogen or
an optionally monohalogen- or polyhalogen-substituted C₁-C₆-alkyl, C₁-C₆-alkoxy or C₃-C₇-cycloalkyl radical.

In formula (I), W and Y independently of one another may preferably represent:

hydrogen, cyano or halogen or
an optionally monohalogen- or polyhalogen-substituted C₁-C₃-alkyl or C₁-C₃-alkoxy radical.

In formula (I), W and Y independently of one another may more preferably represent:

hydrogen or halogen or an optionally monohalogen- or polyhalogen-substituted C₁-C₃-alkyl or C₁-C₃-alkoxy radical.

In formula (I), W and Y independently of one another may particularly preferably represent:

hydrogen or an optionally monohalogen- or polyhalogen-substituted C₁-C₃-alkyl radical.

In formula (I), V¹, V² and V³ independently of one another may represent:

hydrogen, halogen, nitro or cyano or a C₁-C₆-alkyl, halo-C₁-C₆-alkyl, C₁-C₆-alkoxy, halo-C₁-C₆-alkoxy, C₁-C₆-alkylthio, C₁-C₆-alkylsulphinyl, C₁-C₆-alkylsulphonyl, C₁-C₆-alkoxy-C₁-C₆-alkyl, C₃-C₁₀-cycloalkyl or a monocyclic heterocycloalkyl radical, and/or
V¹ and V² together with the carbon atoms to which they are attached form a saturated or unsaturated cycle T¹ which optionally contains at least one further heteroatom and has 4 to 7 ring atoms and whose ring-forming atoms may be mono- or polysubstituted by identical or different substituents selected from the group consisting of halogen and a C₁-C₆-alkyl radical.

In formula (I), V¹, V² and V³ independently of one another may preferably represent:

hydrogen, halogen or cyano or a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical.

In formula (I), V¹, V² and V³ independently of one another may more preferably represent:

hydrogen or halogen or represent a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical.

In formula (I), V¹, V² and V³ independently of one another may particularly preferably represent:

hydrogen, halogen or a C₁-C₃-alkyl or C₁-C₃-haloalkyl radical.

The group CKE may represent one of the following groups:

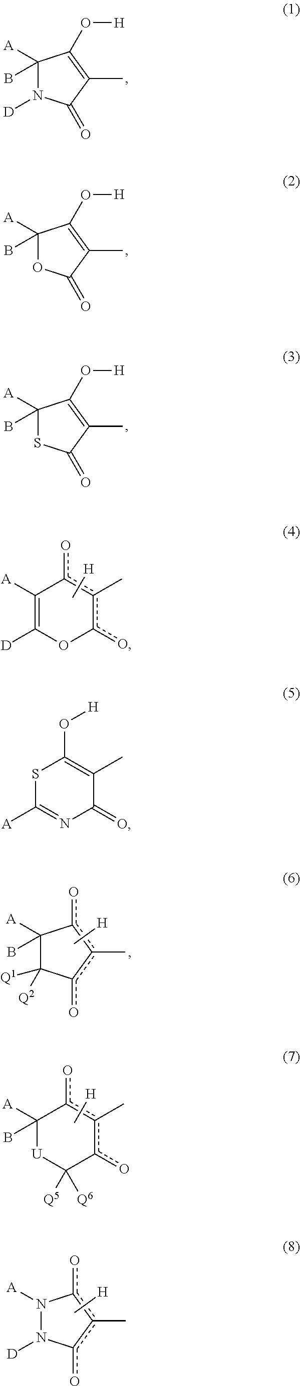

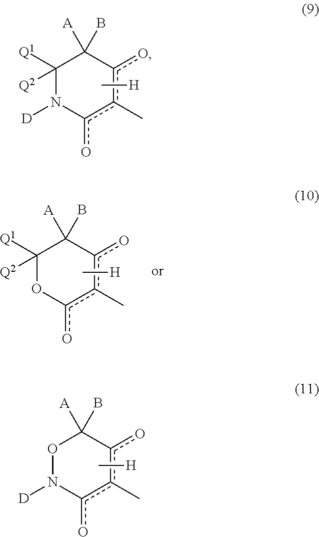

The group CKE may preferably represent one of the following groups:

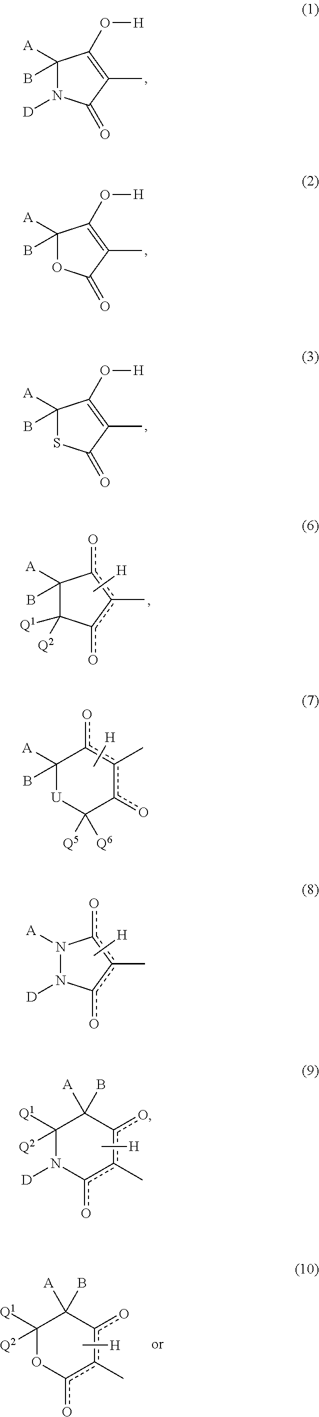

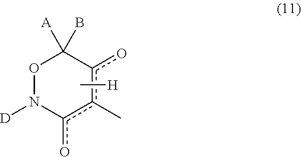

The group CKE may more preferably represent one of the following groups:

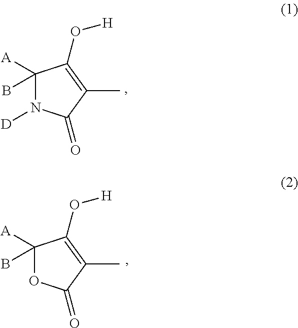

In the group CKE of the formula (I), U may represent:

### —S—, —S(O)—, —S(O)₂—, —O—,

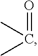

a substituted

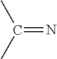

group
or an optionally Q³- and Q⁴-substituted C₁-C₄-alkylene group.

In the group CKE of the formula (I), U may preferably represent an optionally Q³- and Q⁴-substituted methylene group.

In the group CKE of the formula (I), A may represent:

hydrogen or
an optionally monohalogen- or polyhalogen-substituted C₁-C₆-alkyl, C₂-C₆-alkenyl, C₁-C₆-alkoxy-C₁-C₆-alkyl or C₁-C₆-alkylthio-C₁-C₆-alkyl radical or
a C₃-C₇-cycloalkyl or monocyclic heterocyclyl radical, each of which may be mono- or polysubstituted by identical or different substituents selected from the group consisting of halogen and a C₁-C₆-alkyl radical or
an aryl, aryl-C₁-C₆-alkyl or heteroaryl radical, each of which may optionally be mono- or polysubstituted by identical or different substituents selected from the group consisting of halogen, cyano, nitro and C₁-C₆-alkyl, halo-C₁-C₆-alkyl, C₁-C₆-alkoxy and halo-C₁-C₆-alkoxy radicals.

In the group CKE of the formula (I), A may preferably represent:

hydrogen or
an optionally monohalogen- or polyhalogen-substituted C₁-C₆-alkyl or C₁-C₆-alkoxy-C₁-C₆-alkyl radical or
a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical or
a phenyl, phenyl-C₁-C₃-alkyl or monocyclic heteroaryl radical, each of which may optionally be mono- or polysubstituted by identical or different substituents selected from the group consisting of halogen, cyano and C₁-C₃-alkyl, halo-C₁-C₃-alkyl, C₁-C₃-alkoxy and halo-C₁-C₃-alkoxy radicals.

In the group CKE of the formula (I), A may more preferably represent:

hydrogen or
an optionally monohalogen- or polyhalogen-substituted C₁-C₆-alkyl or C₁-C₆-alkoxy-C₁-C₆-alkyl radical or
a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical.

In the group CKE of the formula (I), A may particularly preferably represent:

hydrogen or
an optionally monohalogen- or polyhalogen-substituted C₁-C₆-alkyl or C₁-C₆-alkoxy-C₁-C₆-alkyl radical or
a C₃-C₆-cycloalkyl radical which may be mono- or polysubstituted by identical or different substituents selected from the group consisting of halogen and a C₁-C₃-alkyl radical.

In the group CKE of the formula (I), B may represent:

hydrogen or a C₁-C₆-alkyl or C₁-C₆-alkoxy-C₁-C₆-alkyl radical.

In the group CKE of the formula (I), B may preferably represent:

hydrogen or a C₁-C₆-alkyl or C₁-C₃-alkoxy-C₁-C₃-alkyl radical.

In the group CKE of the formula (I), A and B together with the carbon atom to which they are attached may form:

a saturated or unsaturated cycle T² which optionally contains at least one heteroatom and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen, hydroxyl or cyano or
b) represent a C₁-C₆-alkyl, C₁-C₆-alkoxy, C₁-C₆-alkoxy-C₁-C₆-alkyl, C₁-C₆-alkylcarbonyl, C₁-C₆-alkoxycarbonyl, C₁-C₆-alkylaminocarbonyl, C₁-C₆-alkylthio, C₁-C₆-alkylsulphinyl, C₁-C₆-alkylsulphonyl, C₁-C₆-alkylaminosulphonyl, C₁-C₆-alkoxy-C₁-C₆-alkoxy, halo-C₁-C₆-alkyl or halo-C₁-C₆-alkoxy radical which is optionally hydroxyl-substituted in the alkyl moiety, or
c) represent an aryl, arylcarbonyl, arylsulphonyl, arylamino, heteroaryl, heteroarylcarbonyl, heteroarylsulphonyl or heteroarylamino radical, or
d) represent a C₃-C₇-cycloalkyl, C₃-C₇-cycloalkylcarbonyl, C₃-C₇-cycloalkylsulphonyl, heterocyclyl, heterocyclylcarbonyl or heterocyclylsulphonyl radical, where the radicals mentioned under c) and d) may optionally be mono- or polysubstituted at the ring system by identical or different substituents selected from the group consisting of halogen, hydroxyl, cyano, nitro and halo-C₁-C₆-alkoxy, C₁-C₆-alkoxy-C₁-C₆-alkyl, C₃-C₁₀-cycloalkyl and 3- to 6-membered heterocycloalkyl radicals, and/or
e) two of the radicals R¹, R² and R³ together with the ring atom(s) of the cycle T² to which they are attached may form a further saturated or unsaturated cycle T³ which optionally contains at least one heteroatom and has 3 to 7 ring atoms and which may be mono- or polysubstituted by identical or different substituents selected from the group consisting of the radicals R⁴, R⁵ and R⁶, where R⁴, R⁵ and R⁶ independently of one another represent a C₁-C₆-alkyl or C₁-C₆-alkoxy radical.

In the group CKE of the formula (I), A and B together with the carbon atom to which they are attached may preferably form:

a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₅-alkyl, C₁-C₅-alkoxy, C₁-C₃-alkoxy-C₁-C₃-alkyl, C₁-C₃-alkylcarbonyl, C₁-C₃-alkoxycarbonyl, C₁-C₃-alkylaminocarbonyl, C₁-C₃-alkylthio, C₁-C₃-alkylsulphinyl, C₁-C₃-alkylsulphonyl, C₁-C₃-alkylaminosulphonyl, C₁-C₃-alkoxy-C₁-C₃-alkoxy, halo-C₁-C₃-alkyl or halo-C₁-C₃-alkoxy radical which is optionally hydroxyl-substituted in the alkyl moiety, or
c) represent a phenyl, phenylcarbonyl, phenylsulphonyl or phenylamino radical, or
d) represent a C₃-C₇-cycloalkyl, C₃-C₇-cycloalkylcarbonyl, C₃-C₇-cycloalkylsulphonyl or in each case 4- to 7-membered monocyclic heterocyclyl, heterocyclylcarbonyl or
   heterocyclylsulphonyl radical,
   where the radicals mentioned under c) and d) may optionally be mono- or polysubstituted at the ring system by identical or different substituents selected from the group consisting of halogen, hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, and/or
e) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of the radicals R⁴, R⁵ and R⁶,
   where R⁴, R⁵ and R⁶ independently of one another represent a C₁-C₃-alkyl or C₁-C₃-alkoxy radical.

In the group CKE of the formula (I), A and B together with the carbon atom to which they are attached may more preferably form:

a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety, or
c) represent a C₃-C₇-cycloalkyl or 4- to 7-membered monocyclic heterocyclyl radical, each of which is optionally mono- or polysubstituted by identical or different substituents selected from the group consisting of halogen, hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, and/or
d) two of the radicals R¹, R² and R³ together with the ring atom(s) of the cycle T² to which they are attached may form a further saturated or aromatic cycle T³ which optionally contains one or two heteroatoms and has 5 to 7 ring atoms and which may be mono- or polysubstituted by identical or different substituents selected from the group consisting of the radicals R⁴, R⁵ and R⁶,
   where R⁴, R⁵ and R⁶ independently of one another represent a C₁-C₃-alkyl or C₁-C₃-alkoxy radical.

In the group CKE of the formula (I), A and B together with the carbon atom to which they are attached may particularly preferably form:

a saturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
b) two of the radicals R¹, R² and R³ together with the ring atom(s) of the cycle T² to which they are attached may form a further saturated or aromatic cycle T³ which optionally contains at least one oxygen atom and has 5 to 7 ring atoms and which may be mono- or polysubstituted by a C₁-C₃-alkyl radical.

In the group CKE of the formula (I), D may represent:

hydrogen or
a C₁-C₆-alkyl, C₂-C₆-alkenyl, C₂-C₆-alkynyl or C₁-C₆-alkoxy-C₁-C₆-alkyl radical or
a C₃-C₇-cycloalkyl or monocyclic heterocyclyl radical or
an aryl, aryl-C₁-C₆-alkyl, heteroaryl or heteroaryl-C₁-C₆-alkyl radical,
where the radicals mentioned may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl, C₃-C₁₀-cycloalkyl and monocyclic heterocycloalkyl radicals.

In the group CKE of the formula (I), D may preferably represent:

hydrogen or
a C₁-C₆-alkyl or C₁-C₆-alkoxy-C₁-C₆-alkyl radical or
a C₃-C₇-cycloalkyl or 4- to 7-membered monocyclic heterocyclyl radical or
a phenyl or phenyl-C₁-C₃-alkyl radical,
where the radicals mentioned may optionally be mono- or polysubstituted by identical or different substituents selected from the group consisting of halogen, hydroxyl, cyano and C₁-C₃-alkyl, halo-C₁-C₃-alkyl, C₁-C₃-alkoxy, halo-C₁-C₃-alkoxy, C₁-C₃-alkoxy-C₁-C₃-alkyl and C₃-C₇-cycloalkyl radicals.

In the group CKE of the formula (I), D may more preferably represent:

hydrogen or
a C₁-C₆-alkyl or C₁-C₆-alkoxy-C₁-C₆-alkyl radical or
a C₃-C₇-cycloalkyl or 4- to 7-membered monocyclic heterocyclyl radical,
where the radicals mentioned may optionally be mono- or polysubstituted by identical or different substituents selected from the group consisting of halogen and hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals.

In the group CKE of the formula (I), D may particularly preferably represent:

hydrogen or
a C₁-C₆-alkyl or C₃-C₇-cycloalkyl radical,
where the radicals mentioned may be mono- or polysubstituted by identical or different substituents selected from the group consisting of halogen, hydroxyl and a C₁-C₃-alkyl radical.

In the group CKE of the formula (I), A and D together with the atoms to which they are attached may alternatively form:

a saturated or unsaturated cycle T⁴ which optionally contains at least one further heteroatom and has 3 to 7 ring atoms, which may be bridged and whose ring-forming atoms may be mono- or polysubstituted by identical or different substituents selected from the group consisting of the radicals R⁷, R⁸ and R⁹,
where R⁷, R⁸ and R⁹ independently of one another represent hydroxyl, halogen or a C₁-C₆-alkyl or C₁-C₆-alkoxy radical.

If the group CKE of the formula (I) is the group 8, A and D together with the atoms to which they are attached may alternatively preferably form:

a saturated or unsaturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
where R⁷, R⁸ and R⁹ independently of one another represent halogen or a C₁-C₃-alkyl or C₁-C₃-alkoxy radical.

If the group CKE of the formula (I) is the group 8, A and D together with the atoms to which they are attached may alternatively more preferably form:

a saturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
where R⁷, R⁸ and R⁹ independently of one another represent halogen or a C₁-C₃-alkyl radical.

In the group CKE of the formula (I), A and Q¹ together with the atoms to which they are attached may form:

a saturated or unsaturated cycle T⁵ which optionally contains at least one further heteroatom and has 5 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals,
with the proviso that B and Q² represent a bond if the cycle T⁵ formed by A and Q¹ is aromatic.

In the group CKE of the formula (I), A and Q¹ together with the atoms to which they are attached may preferably form:

an unsaturated cycle T⁵ which optionally contains at least one further heteroatom and has 5 to 7 ring atoms and whose ring-forming atoms may be mono- or polysubstituted by identical or different substituents selected from the group consisting of halogen and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals,
with the proviso that B and Q² represent a bond if the cycle T⁵ formed by A and Q¹ is aromatic.

In the group CKE of the formula (I), A and Q¹ together with the atoms to which they are attached may particularly preferably form:

an aromatic cycle T⁵ which has 6 ring atoms and whose ring-forming atoms may be mono- or polysubstituted by halogen,
with the proviso that in this case B and Q² represent a bond.

In the group CKE of the formula (I), Q¹ may represent:

hydrogen or
a C₁-C₆-alkyl or C₁-C₆-alkoxy radical which is optionally mono- or polysubstituted by identical or different substituents selected from the group consisting of halogen, hydroxyl and a C₁-C₆-alkoxy radical, or
a C₃-C₇-cycloalkyl or monocyclic heterocyclyl radical, each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals, or
a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals.

In the group CKE of the formula (I), Q¹ may preferably represent:

hydrogen or
a C₁-C₃-alkyl or C₁-C₃-alkoxy radical which is optionally mono- or polysubstituted by identical or different substituents selected from the group consisting of halogen, hydroxyl and a C₁-C₆-alkoxy radical, or
a C₃-C₇-cycloalkyl or 4- to 7-membered monocyclic heterocyclyl radical, each of which may optionally be mono- or polysubstituted by identical or different substituents selected from the group consisting of halogen, hydroxyl and C₁-C₃-alkyl, halo-C₁-C₃-alkyl, C₁-C₃-alkoxy, C₁-C₃-alkoxy-C₁-C₃-alkoxy, and halo-C₁-C₃-alkoxy radicals.

In the group CKE of the formula (I), Q¹ may particularly preferably represent:

hydrogen or a C₁-C₃-alkyl radical.

In the group CKE of the formula (I), Q², Q⁴Q⁵ and Q⁶ independently of one another may represent hydrogen or a C₁-C₆-alkyl radical.

In the group CKE of the formula (I), Q², Q⁴Q⁵ and Q⁶ independently of one another may preferably represent hydrogen or a C₁-C₃-alkyl radical.

In the group CKE of the formula (I), Q³ may represent:

hydrogen or
a C₁-C₆-alkyl or C₁-C₆-alkoxy radical which is optionally mono- or polysubstituted by identical or different substituents selected from the group consisting of halogen, hydroxyl and a C₁-C₆-alkoxy radical, or
a C₃-C₇-cycloalkyl or monocyclic heterocyclyl radical, each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals, or
represent a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals.

In the group CKE of the formula (I), Q³ may preferably represent:

hydrogen or
a C₁-C₃-alkyl or C₁-C₃-alkoxy radical which is optionally mono- or polysubstituted by identical or different substituents selected from the group consisting of halogen, hydroxyl and a C₁-C₆-alkoxy radical.

In the group CKE of the formula (I), Q³ may particularly preferably represent:

hydrogen or a C₁-C₃-alkyl radical.

In the group CKE of the formula (I), Q¹ and Q² together with the carbon atom to which they are attached may form a saturated or unsaturated cycle T⁶ which optionally contains at least one further heteroatom and has 3 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals.

In the group CKE of the formula (I), Q³ and Q⁴ together with the carbon atom to which they are attached may form:

a saturated or unsaturated cycle T⁷ which optionally contains at least one heteroatom and has 3 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals.

In the group CKE of the formula (I), Q³ and Q⁴ together with the carbon atom to which they are attached may preferably form:

a saturated cycle T⁷ which optionally contains at least one heteroatom and has 5 to 7 ring atoms and whose ring-forming atoms may be mono- or polysubstituted by identical or different substituents selected from the group consisting of halogen, hydroxyl and C₁-C₃-alkyl, halo-C₁-C₃-alkyl, C₁-C₆-alkoxy, halo-C₁-C₃-alkoxy and C₁-C₃-alkoxy-C₁-C₃-alkyl radicals.

In the group CKE of the formula (I), Q³ and Q⁴ together with the carbon atom to which they are attached may particularly preferably form:

a saturated cycle T⁷ which has 6 ring atoms and whose ring-forming atoms may be mono- or polysubstituted by identical or different substituents selected from the group consisting of halogen and a C₁-C₆-alkyl radical.

A preferred group of compounds of the general formula (I) is formed by compounds of the general formula (I)

in which
X represents halogen or an optionally monohalogen- or polyhalogen-substituted C₁-C₃-alkyl or C₁-C₃-alkoxy radical, and
W and Y independently of one another represent hydrogen, cyano or halogen or an optionally monohalogen- or polyhalogen-substituted C₁-C₃-alkyl or C₁-C₃-alkoxy radical, and
V¹, V² and V³ independently of one another represent hydrogen, halogen or cyano or a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
CKE represents one of the groups

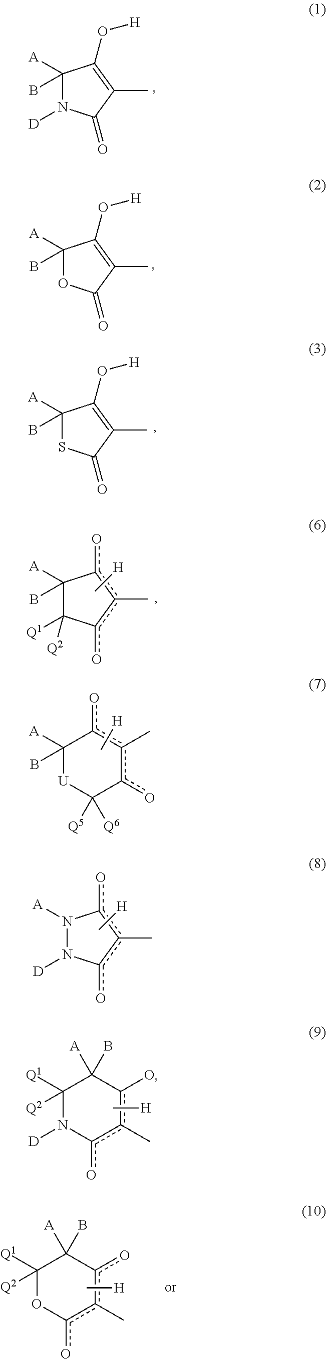

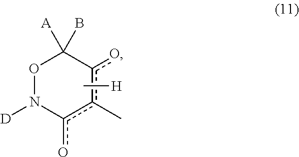

in which
U represents an optionally Q³- and Q⁴-substituted methylene group, and
A represents hydrogen or
   an optionally monohalogen- or polyhalogen-substituted C₁-C₆-alkyl or C₁-C₆-alkoxy-C₁-C₆-alkyl radical or
   a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, or
   a phenyl, phenyl-C₁-C₃-alkyl or monocyclic heteroaryl radical, each of which may be mono- or polysubstituted by identical or different substituents selected from the group consisting of halogen, cyano and C₁-C₃-alkyl, halo-C₁-C₃-alkyl, C₁-C₃-alkoxy and halo-C₁-C₃-alkoxy radicals, and
B represents hydrogen or represents a C₁-C₆-alkyl or C₁-C₆-alkoxy-C₁-C₆-alkyl radical, or
A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen or hydroxyl or
   b) represent a C₁-C₅-alkyl, C₁-C₅-alkoxy, C₁-C₃-alkoxy-C₁-C₃-alkyl, C₁-C₃-alkylcarbonyl, C₁-C₃-alkoxycarbonyl, C₁-C₃-alkylaminocarbonyl, C₁-C₃-alkylthio, C₁-C₃-alkylsulphinyl, C₁-C₃-alkylsulphonyl, C₁-C₃-alkylaminosulphonyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
   c) represent a phenyl, phenylcarbonyl, phenylsulphonyl or phenylamino radical, or
   d) represent a C₃-C₇-cycloalkyl, C₃-C₇-cycloalkylcarbonyl, C₃-C₇-cycloalkylsulphonyl or in each case 4- to 7-membered monocyclic heterocyclyl, heterocyclylcarbonyl or heterocyclylsulphonyl radical, where the radicals mentioned under c) and d) may optionally be mono- or polysubstituted at the ring system by identical or different substituents selected from the group consisting of halogen, hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, and/or
   e) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of the radicals R⁴, R⁵ and R⁶,
      where R⁴, R⁵ and R⁶ independently of one another represent a C₁-C₃-alkyl or C₁-C₃-alkoxy radical, and
D represents hydrogen or
   represents a C₁-C₆-alkyl or C₁-C₆-alkoxy-C₁-C₆-alkyl radical or
   represents a C₃-C₇-cycloalkyl or 4- to 7-membered monocyclic heterocyclyl radical or
   represents a phenyl or phenyl-C₁-C₃-alkyl radical,
   where the radicals mentioned may optionally be mono- or polysubstituted by identical or different substituents selected from the group consisting of halogen, hydroxyl, cyano and C₁-C₃-alkyl, halo-C₁-C₃-alkyl, C₁-C₃-alkoxy, halo-C₁-C₃-alkoxy, C₁-C₃-alkoxy-C₁-C₃-alkyl and C₃-C₇-cycloalkyl radicals, or
if CKE is the group 8,
A and D alternatively together with the atoms to which they are attached form a saturated or unsaturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
   where R⁷, R⁸ and R⁹ independently of one another represent halogen or a C₁-C₃-alkyl or C₁-C₃-alkoxy radical, and
A and Q¹ together with the atoms to which they are attached form an unsaturated cycle T⁵ which optionally contains at least one further heteroatom and has 5 to 7 ring atoms and whose ring-forming atoms may be mono- or polysubstituted by identical or different substituents selected from the group consisting of halogen and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals,
   with the proviso that B and Q² represent a bond if the cycle T⁵ formed by A and Q¹ is aromatic, and
Q¹ represents hydrogen or
   represents a C₁-C₃-alkyl or C₁-C₃-alkoxy radical which may optionally be mono- or polysubstituted by identical or different substituents selected from the group consisting of halogen, hydroxyl and a C₁-C₆-alkoxy radical or
   represents a C₃-C₇-cycloalkyl or 4- to 7-membered monocyclic heterocyclyl radical, each of which may optionally be mono- or polysubstituted by identical or different substituents selected from the group consisting of halogen, hydroxyl and C₁-C₃-alkyl, halo-C₁-C₃-alkyl, C₁-C₃-alkoxy, C₁-C₃-alkoxy-C₁-C₃-alkoxy and halo-C₁-C₃-alkoxy radicals, and
Q², Q⁴, Q⁵ and Q⁶ independently of one another represent hydrogen or represent a C₁-C₃-alkyl radical, and
Q³ represents hydrogen or
   represents a C₁-C₃-alkyl or C₁-C₃-alkoxy radical which may optionally be mono- or polysubstituted by identical or different substituents selected from the group consisting of halogen, hydroxyl and a C₁-C₆-alkoxy radical, or
Q³ and Q⁴ together with the carbon atom to which they are attached form a saturated cycle T⁷ which optionally contains at least one heteroatom and has 5 to 7 ring atoms and whose ring-forming atoms may be mono- or polysubstituted by identical or different substituents selected from the group consisting of halogen, hydroxyl and C₁-C₃-alkyl, halo-C₁-C₃-alkyl, C₁-C₆-alkoxy, halo-C₁-C₃-alkoxy and C₁-C₃-alkoxy-C₁-C₃-alkyl radicals.

A more preferred group of compounds of the general formula (I) is formed by compounds of the general formula (I),

in which
X represents halogen or represents an optionally monohalogen- or polyhalogen-substituted C₁-C₃-alkyl or C₁-C₃-alkoxy radical and
W and Y independently of one another represent hydrogen or halogen or represent an optionally monohalogen- or polyhalogen-substituted C₁-C₃-alkyl or C₁-C₃-alkoxy radical, and
V¹, V² and V³ independently of one another represent hydrogen or halogen or represent a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
CKE represents one of the groups

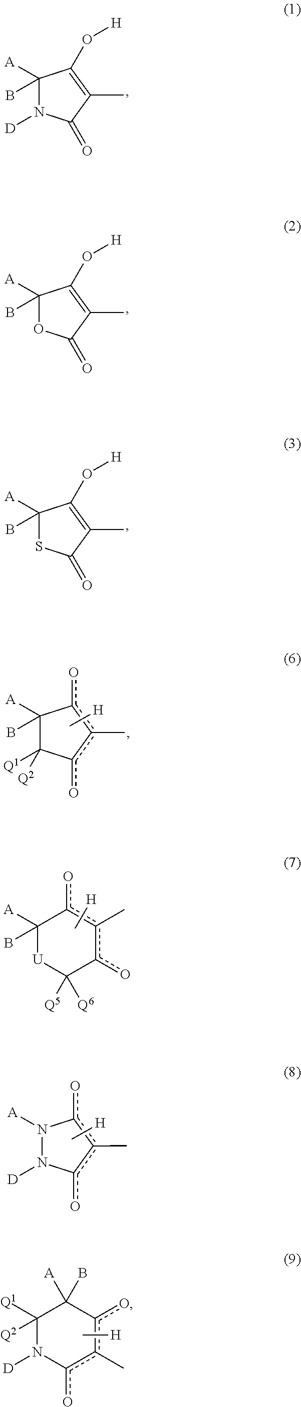

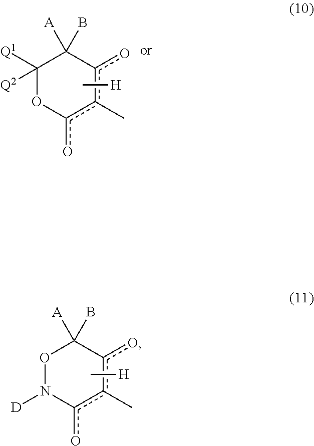

in which
U represents an optionally Q³- and Q⁴-substituted methylene group, and
A represents hydrogen or
   represents an optionally monohalogen- or polyhalogen-substituted C₁-C₆-alkyl or C₁-C₆-alkoxy-C₁-C₆-alkyl radical or represents a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, and
B represents hydrogen or represents a C₁-C₆-alkyl or C₁-C₃-alkoxy-C₁-C₃-alkyl radical, or
A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen or hydroxyl or
   b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety or
   c) represent a C₃-C₇-cycloalkyl or 4- to 7-membered monocyclic heterocyclyl radical, each of which may optionally be mono- or polysubstituted in the ring system by identical or different substituents selected from the group consisting of halogen, hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, and/or
   d) two of the radicals R¹, R² and R³ together with the ring atom(s) of the cycle T² to which they are attached may form a saturated or aromatic cycle T³ which optionally contains one or two heteroatoms and has 5 to 7 ring atoms and which may be mono- or polysubstituted by identical or different substituents selected from the group consisting of the radicals R⁴, R⁵ and R⁶,
      where R⁴, R⁵ and R⁶ independently of one another represent a C₁-C₃-alkyl or C₁-C₃-alkoxy radical, and
D represents hydrogen or
   represents a C₁-C₆-alkyl or C₁-C₆-alkoxy-C₁-C₆-alkyl radical or
   represents a C₃-C₇-cycloalkyl or 4- to 7-membered monocyclic heterocyclyl radical,
   where the radicals mentioned may optionally be mono- or polysubstituted by identical or different substituents selected from the group consisting of halogen and hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, or
if CKE is the group 8,
A and D alternatively together with the atoms to which they are attached form a saturated or unsaturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
   where R⁷, R⁸ and R⁹ independently of one another represent halogen or a C₁-C₃-alkyl or C₁-C₃-alkoxy radical, and
A and Q¹ together with the atoms to which they are attached form an unsaturated cycle T⁵ which optionally contains at least one further heteroatom and has 5 to 7 ring atoms and whose ring-forming atoms may be mono- or polysubstituted by identical or different substituents selected from the group consisting of halogen and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals,
   with the proviso that B and Q² represent a bond if the cycle T⁵ formed by A and Q¹ is aromatic, and
Q¹ represents hydrogen or
   represents a C₁-C₃-alkyl or C₁-C₃-alkoxy radical which may optionally be mono- or polysubstituted by identical or different substituents selected from the group consisting of halogen, hydroxyl and a C₁-C₆-alkoxy radical or
   represents a C₃-C₇-cycloalkyl or 4- to 7-membered monocyclic heterocyclyl radical, each of which may optionally be mono- or polysubstituted by identical or different substituents selected from the group consisting of halogen, hydroxyl and C₁-C₃-alkyl, halo-C₁-C₃-alkyl, C₁-C₃-alkoxy, C₁-C₃-alkoxy-C₁-C₃-alkoxy and halo-C₁-C₃-alkoxy radicals, and
Q², Q⁴, Q⁵ and Q⁶ independently of one another represent hydrogen or represent a C₁-C₃-alkyl radical, and
Q³ represents hydrogen or
   represents a C₁-C₃-alkyl or C₁-C₃-alkoxy radical which may optionally be mono- or polysubstituted by identical or different substituents selected from the group consisting of halogen, hydroxyl and a C₁-C₆-alkoxy radical, or
Q³ and Q⁴ together with the carbon atom to which they are attached form a saturated cycle T⁷ which optionally contains at least one heteroatom and has 5 to 7 ring atoms and whose ring-forming atoms may be mono- or polysubstituted by identical or different substituents selected from the group consisting of halogen, hydroxyl and C₁-C₃-alkyl, halo-C₁-C₃-alkyl, C₁-C₆-alkoxy, halo-C₁-C₃-alkoxy and C₁-C₃-alkoxy-C₁-C₃-alkyl radicals.

A particularly preferred sub-group is formed by compounds of the general formula (I) in which
X represents halogen or an optionally monohalogen- or polyhalogen-substituted C₁-C₃-alkyl radical, and
W and Y independently of one another represent hydrogen or an optionally monohalogen- or polyhalogen-substituted C₁-C₃-alkyl radical,
V¹, V² and V³ independently of one another represent hydrogen, halogen or a C₁-C₃-alkyl or C₁-C₃-haloalkyl radical, and
CKE represents one of the groups

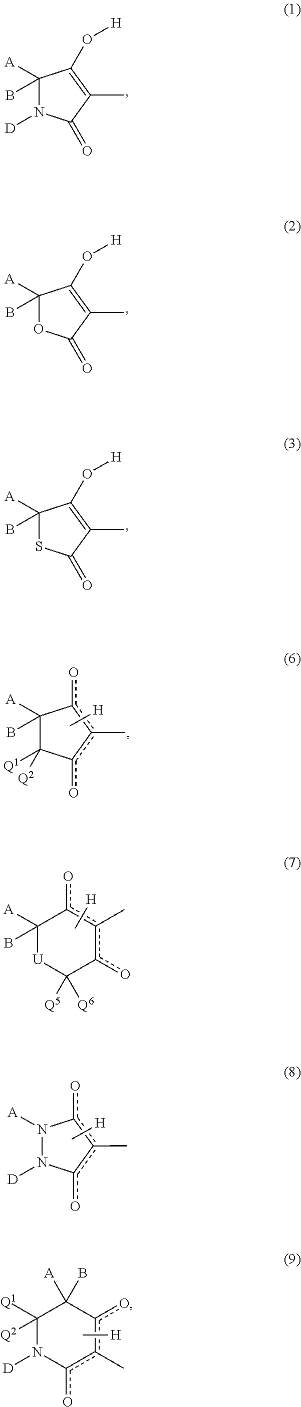

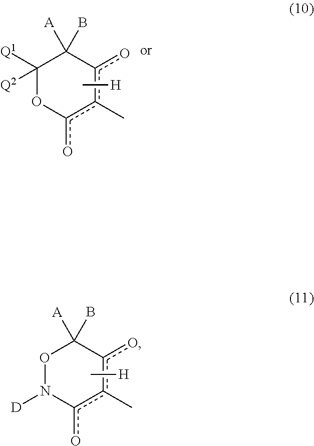

in which
U represents an optionally Q³- and Q⁴-substituted methylene group,
A represents hydrogen or
   represents an optionally monohalogen- or polyhalogen-substituted C₁-C₆-alkyl or C₁-C₆-alkoxy-C₁-C₆-alkyl radical or
   represents a C₃-C₆-cycloalkyl radical which may be mono- or polysubstituted by identical or different substituents selected from the group consisting of halogen and a C₁-C₃-alkyl radical, and
B represents hydrogen or a C₁-C₆-alkyl or C₁-C₃-alkoxy-C₁-C₃-alkyl radical, or
A and B together with the carbon atom to which they are attached form a saturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally substituted in the alkyl moiety by hydroxyl and/or
   b) two of the radicals R¹, R² and R³ together with the ring atom(s) of the cycle T² to which they are attached may form a further saturated or aromatic cycle T³ which optionally contains at least one oxygen atom and has 5 to 7 ring atoms and which may be mono- or polysubstituted by a C₁-C₃-alkyl radical, and
D represents hydrogen or a C₁-C₆-alkyl or C₃-C₇-cycloalkyl radical,
   where the radicals mentioned may be mono- or polysubstituted by identical or different substituents selected from the group consisting of halogen, hydroxyl and a C₁-C₃-alkyl radical, or
if CKE is the group 8,
A and D alternatively together with the atoms to which they are attached form a saturated or unsaturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
   where R⁷, R⁸ and R⁹ independently of one another represent halogen or a C₁-C₃-alkyl radical, or
A and Q¹ together with the atoms to which they are attached form an aromatic cycle T⁵ which has 6 ring atoms and whose ring-forming atoms may be mono- or polysubstituted by halogen,
   with the proviso that in this case B and Q² represent a bond, and
Q¹ represents hydrogen or a C₁-C₃-alkyl radical, and
Q², Q⁴, Q⁵ and Q⁶ independently of one another represent hydrogen or represent a C₁-C₃-alkyl radical, and
Q³ represents hydrogen or represents a C₁-C₃-alkyl radical, or
Q³ and Q⁴ together with the carbon atom to which they are attached form a saturated cycle T⁷ which has 6 ring atoms and whose ring-forming atoms may be mono- or polysubstituted by identical or different substituents selected from the group consisting of halogen and a C₁-C₆-alkyl radical.

Within the group of compounds of the general formula (I), depending on the CKE group, the following sub-groups result:

compounds of the general formula (I-1)

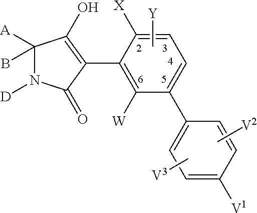

compounds of the general formula (1-2)

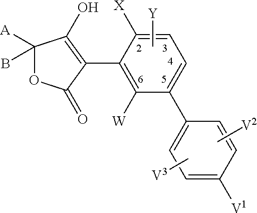

compounds of the general formula (I-3)

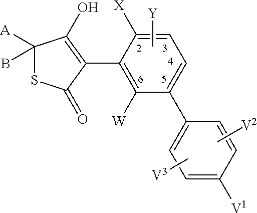

compounds of the general formula (1-4)

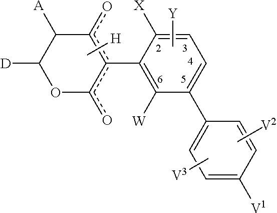

compounds of the general formula (1-5)

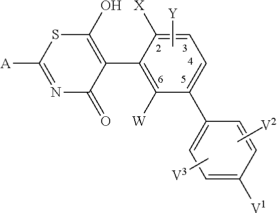

compounds of the general formula (I-6)

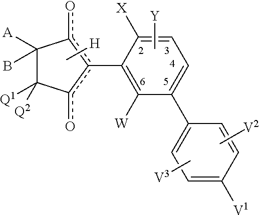

compounds of the general formula (1-7)

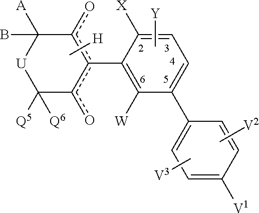

compounds of the general formula (1-8)

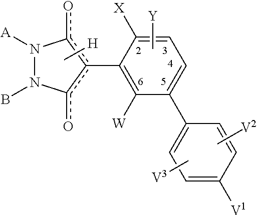

compounds of the general formula (I-9)

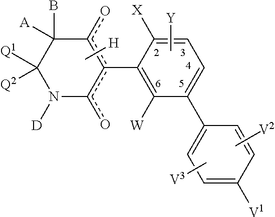

compounds of the general formula (1-10)

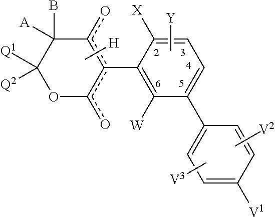

compounds of the general formula (1-11)

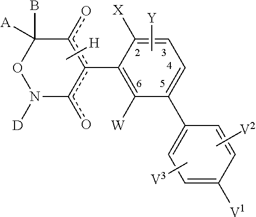

A preferred sub-group is formed by compounds of the general formula (I-1),

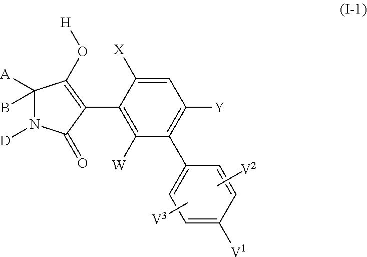

in which
X represents halogen or
   represents an optionally monohalogen- or polyhalogen-substituted C₁-C₃-alkyl or C₁-C₃-alkoxy radical, and
W and Y independently of one another represent hydrogen or halogen or represent an optionally monohalogen- or polyhalogen-substituted C₁-C₃-alkyl radical, and
V¹, V² and V³ independently of one another represent hydrogen or halogen or represent a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
A represents hydrogen or
   represents an optionally monohalogen- or polyhalogen-substituted C₁-C₆-alkyl or C₁-C₆-alkoxy-C₁-C₆-alkyl radical or
   represents a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, and
B represents hydrogen or represents a C₁-C₆-alkyl or C₁-C₃-alkoxy-C₁-C₃-alkyl radical, or
A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen or hydroxyl or
   b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
   c) two of the radicals R¹, R² and R³ together with the ring atom(s) of the cycle T² to which they are attached may form a further saturated or aromatic cycle T³ which optionally contains one or two heteroatoms and has 5 to 7 ring atoms and which may be mono- or polysubstituted by identical or different substituents selected from the group consisting of the radicals R⁴, R⁵ and R⁶,
      where R⁴, R⁵ and R⁶ independently of one another represent a C₁-C₃-alkyl or C₁-C₃-alkoxy radical, and
D represents hydrogen or
   represents a C₁-C₆-alkyl or C₁-C₆-alkoxy-C₁-C₆-alkyl radical or
   represents a C₃-C₇-cycloalkyl or 4- to 7-membered monocyclic heterocyclyl radical, where the radicals mentioned may optionally be mono- or polysubstituted by identical or different substituents selected from the group consisting of halogen and hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, or
A and D together with the atoms to which they are attached form a saturated or unsaturated cycle T⁴ which optionally contains a further heteroatom and has 5 to 7 ring atoms and whose ring-forming atoms may be mono- or polysubstituted by identical or different substituents selected from the group consisting of the radicals R⁷, R⁸ and R⁹,
   where R⁷, R⁸ and R⁹ independently of one another represent halogen or a C₁-C₃-alkyl or C₁-C₃-alkoxy radical.

In formula (I-1), X may represent:

halogen or an optionally monohalogen- or polyhalogen-substituted C₁-C₃-alkyl or C₁-C₃-alkoxy radical.

In formula (I-1), X may preferably represent:

chlorine or a methyl radical.

In formula (I-1), W and Y independently of one another may represent:

hydrogen or halogen or an optionally monohalogen- or polyhalogen-substituted C₁-C₃-alkyl radical.

In formula (I-1), W and Y independently of one another may preferably represent:

hydrogen or a methyl radical.

In formula (I-1), X, W and Y independently of one another may with extraordinary preference represent:

X represents methyl and W and Y represent hydrogen or
X and W represent methyl and Y represents hydrogen or
X and Y represent methyl and W represents hydrogen.

In formula (I-1), V¹, V² and V³ independently of one another may represent:

hydrogen or halogen or
a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,

In formula (I-1), V¹, V² and V³ independently of one another may preferably represent:

hydrogen, chlorine or fluorine or a methyl or a trifluoromethyl radical.

In formula (I-1), V¹, V² and V³ may more preferably represent:

V¹ represents hydrogen, chlorine or fluorine or represents a methyl or a trifluoromethyl radical, and
V² and V³ independently of one another represent hydrogen, chlorine or fluorine.

In formula (I-1), V¹, V² and V³ may particularly preferably represent:

V¹ represents chlorine, fluorine or a methyl radical and
V² and V³ independently of one another represent hydrogen, chlorine or fluorine.

In formula (I-1), V¹ may with extraordinary preference represent chlorine or fluorine, in particular chlorine.

In formula (I-1), A may represent:

hydrogen or
an optionally monohalogen- or polyhalogen-substituted C₁-C₆-alkyl or C₁-C₆-alkoxy-C₁-C₆-alkyl radical or
a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical.

In formula (I-1), A may preferably represent:

hydrogen or
an optionally halogen-substituted C₁-C₄-alkyl or C₁-C₃-alkoxy-C₁-C₃-alkyl radical or a C₃-C₆-cycloalkyl radical which may be mono- or polysubstituted by identical or different substituents selected from the group consisting of halogen and a C₁-C₃-alkyl radical.

In formula (I-1), A may more preferably represent:

hydrogen or
an optionally halogen-substituted C₁-C₄-alkyl or C₁-C₃-alkoxy-C₁-C₃-alkyl radical or a C₃-C₆-cycloalkyl radical.

In formula (I-1), A may particularly preferably represent:

hydrogen or
a C₁-C₄-alkyl, methoxy-C₁-C₂-alkyl or a C₃-C₆-cycloalkyl radical.

In formula (I-1), B may represent:

hydrogen or a C₁-C₆-alkyl or C₁-C₃-alkoxy-C₁-C₃-alkyl radical.

In formula (I-1), B may preferably represent:

hydrogen or a C₁-C₃-alkyl radical.

In formula (I-1), B may more preferably represent:

hydrogen or a methyl radical.

In formula (I-1), A and B may together with the carbon atom to which they are attached form:

a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
c) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of the radicals R⁴, R⁵ and R⁶,
   where R⁴, R⁵ and R⁶ independently of one another represent a C₁-C₃-alkyl or C₁-C₃-alkoxy radical.

In formula (I-1), A and B together with the carbon atom to which they are attached may preferably form:

a saturated cycle T² which optionally contains one or two heteroatoms and has 3 to 8 ring atoms and whose ring-forming atoms may be mono- or disubstituted by identical or different substituents selected from the group consisting of the radicals R¹, R² and R³, where R¹, R² and R³ independently of one another
a) represent a C₁-C₄-alkyl, C₁-C₄-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
b₁) two of the radicals R¹, R² and R³ together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
b₂) two of the radicals R¹, R² and R³ together with the adjacent ring atoms of the cycle T² to which they are attached may form a further aromatic cycle T³ which has 6 ring atoms and may be mono- or disubstituted by a C₁-C₃-alkyl radical.

In formula (I-1), A and B together with the carbon atom to which they are attached may more preferably form:

a saturated cycle T² which optionally contains one heteroatom and has 3 to 8 ring atoms and whose ring-forming atoms may be mono- or disubstituted by identical or different substituents selected from the group consisting of the radicals R¹, R² and R³, where R¹, R² and R³ independently of one another
a) represent a C₁-C₄-alkyl, C₁-C₄-alkoxy, C₁-C₃-alkoxy-C₁-C₃-alkyl, C₁-C₃-alkoxy-C₁-C₃-alkoxy or halo-C₁-C₃-alkoxy radical, and/or
b₁) two of the radicals R¹, R² and R³ together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
b₂) two of the radicals R¹, R² and R³ together with the adjacent ring atoms of the cycle T² to which they are attached may form a further aromatic cycle T³ which has 6 ring atoms.

In formula (I-1), A and B together with the carbon atom to which they are attached may likewise more preferably form:

a saturated cycle T² which optionally contains one oxygen atom and has 3 to 8 ring atoms and whose ring-forming atoms may be mono- or disubstituted by identical or different substituents selected from the group consisting of the radicals R¹, R² and R³, where R¹, R² and R³ independently of one another
a) represent a C₁-C₃-alkyl, C₁-C₄-alkoxy, methoxy-C₁-C₃-alkyl, C₁-C₂-alkoxyethoxy, 2,2,2-trifluoroethoxy radical and/or
b₁) two of the radicals R¹, R² and R³ together with the ring atom of the cycle T² to which they are attached may form a further saturated cycle T³ which optionally contains one or two oxygen atoms and has 5 or 6 ring atoms and may be mono- or disubstituted by a methyl radical, or
b₁) two of the radicals R¹, R² and R³ together with the adjacent ring atoms of the cycle T² to which they are attached may form a further aromatic cycle T³ which has 6 ring atoms.

In formula (I-1), A and B together with the carbon atom to which they are attached may particularly preferably form:

a saturated cycle T² which optionally contains one oxygen atom and has 5 to 6 ring atoms and whose ring-forming atoms may be mono- or disubstituted by identical or different substituents selected from the group consisting of the radicals R¹, R² and R³, where R¹, R² and R³ independently of one another
a) represent a C₁-C₃-alkyl, C₁-C₄-alkoxy, methoxy-C₁-C₂-alkyl, 2,2,2-trifluoroethoxy radical and/or
b) two of the radicals R¹, R² and R³ together with the ring atom of the cycle T² to which they are attached may form a further saturated cycle T³ which optionally contains one or two oxygen atoms and has 5 or 6 ring atoms and may be mono- or disubstituted by a methyl radical.

In formula (I-1), A and B together with the carbon atom to which they are attached may with extraordinary preference form:

a saturated cycle T² which optionally contains one oxygen atom and has 6 ring atoms and whose ring-forming atoms may be mono- or disubstituted by identical or different substituents selected from the group consisting of the radicals R¹, R² and R³,
where R¹, R² and R³ independently of one another represent a C₁-C₃-alkyl, C₁-C₄-alkoxy, methoxy-C₁-C₂-alkyl, 2,2,2-trifluoroethoxy radical.

In formula (I-1), A and B together with the carbon atom to which they are attached may very preferably form a cyclohexane ring or tetrahydropyran ring.

Any substituents R¹, R² and R³ of the cycle T² formed by A and B very preferably independently of one another represent a C₁-C₃-alkyl, C₁-C₂-alkoxy, methoxy-C₁-C₂-alkyl or 2,2,2-trifluoroethoxy radical.

Particular preference is given to only one substituent, where R¹ represents a C₁-C₃-alkyl, C₁-C₂-alkoxy, methoxy-C₁-C₂-alkyl or 2,2,2-trifluoroethoxy radical.

In formula (I-1), D may represent:

hydrogen or
a C₁-C₆-alkyl or C₁-C₆-alkoxy-C₁-C₆-alkyl radical or
a C₃-C₇-cycloalkyl or 4- to 7-membered monocyclic heterocyclyl radical,
where the radicals mentioned may optionally be mono- or polysubstituted by identical or different substituents selected from the group consisting of halogen and/or hydroxyl and C₁-C₃-alkyl, halo-C₁-C₃-alkyl, C₁-C₃-alkoxy, halo-C₁-C₃-alkoxy or C₁-C₃-alkoxy-C₁-C₃-alkyl radicals.

In formula (I-1), D may preferably represent:

hydrogen or a C₁-C₆-alkyl or C₃-C₇-cycloalkyl radical.

In formula (I-1), D may more preferably represent:

hydrogen or a C₁-C₄-alkyl or C₃-C₆-cycloalkyl radical.

In formula (I-1), D may particularly preferably represent:

hydrogen.

In formula (I-1), A and D together with the atoms to which they are attached may form:

a saturated or unsaturated cycle T⁴ which optionally contains a further heteroatom and has 5 to 7 ring atoms and whose ring-forming atoms may be mono- or polysubstituted by identical or different substituents selected from the group consisting of the radicals R⁷, R⁸ and R⁹, where R⁷, R⁸ and R⁹ independently of one another represent halogen or a C₁-C₃-alkyl or C₁-C₃-alkoxy radical.

In formula (I-1), A and D together with the atoms to which they are attached may preferably form:

a saturated cycle T⁴ which optionally contains a further heteroatom and has 5 to 7 ring atoms.

In formula (I-1), A and D together with the atoms to which they are attached may more preferably form:

a saturated cycle T⁴ which optionally contains sulphur as a further heteroatom and has 5 to 7 ring atoms.

In formula (I-1), A and D together with the atoms to which they are attached may particularly preferably form:

a saturated cycle T⁴ which optionally contains sulphur as a further heteroatom and has 6 ring atoms.

A preferred sub-group of the compound of the general formula (I-1) is formed by compounds in which
X represents chlorine or represents a methyl radical, and
W and Y independently of one another represent hydrogen or represent a methyl radical,
V¹, V² and V³ independently of one another represent hydrogen, chlorine or fluorine or represent a methyl or a trifluoromethyl radical, and
A represents hydrogen or represents an optionally halogen-substituted C₁-C₄-alkyl or C₁-C₃-alkoxy-C₁-C₃-alkyl radical or
   represents a C₃-C₆-cycloalkyl radical which may be mono- or polysubstituted by identical or different substituents selected from the group consisting of halogen and a C₁-C₃-alkyl radical, and
B represents hydrogen or represents a C₁-C₃-alkyl radical, or
A and B together with the carbon atom to which they are attached form a saturated cycle T² which optionally contains one or two heteroatoms and has 3 to 8 ring atoms and whose ring-forming atoms may be mono- or disubstituted by identical or different substituents selected from the group consisting of the radicals R¹, R² and R³, where R¹, R² and R³ independently of one another
   a) represent a C₁-C₄-alkyl, C₁-C₄-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
   b₁) two of the radicals R¹, R² and R³ together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
   b₂) two of the radicals R¹, R² and R³ together with the adjacent ring atoms of the cycle T² to which they are attached may form a further aromatic cycle T³ which has 6 ring atoms and may be mono- or disubstituted by a C₁-C₃-alkyl radical, and
D represents hydrogen or represents a C₁-C₆-alkyl or C₃-C₇-cycloalkyl radical.

A more preferred sub-group is formed by compounds of the general formula (I-1), in which
X represents chlorine or represents a methyl radical, and
W and Y independently of one another represent hydrogen or represent a methyl radical,
V¹, V² and V³ independently of one another represent hydrogen, chlorine or fluorine or represent a methyl or a trifluoromethyl radical, and
A represents hydrogen or represents an optionally halogen-substituted C₁-C₄-alkyl or C₁-C₃-alkoxy-C₁-C₃-alkyl radical or
   represents a C₃-C₆-cycloalkyl radical, and
B represents hydrogen or represents a C₁-C₃-alkyl radical, or
A and B together with the carbon atom to which they are attached form a saturated cycle T² which optionally contains one heteroatom and has 3 to 8 ring atoms and whose ring-forming atoms may be mono- or disubstituted by identical or different substituents selected from the group consisting of the radicals R¹, R² and R³, where R¹, R² and R³ independently of one another
   a) represent a C₁-C₄-alkyl, C₁-C₄-alkoxy, C₁-C₃-alkoxy-C₁-C₃-alkyl, C₁-C₃-alkoxy-C₁-C₃-alkoxy or halo-C₁-C₃-alkoxy radical, and/or
   b₁) two of the radicals R¹, R² and R³ together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
   b₂) two of the radicals R¹, R² and R³ together with the adjacent ring atoms of the cycle T² to which they are attached may form a further aromatic cycle T³ which has 6 ring atoms, and
D represents hydrogen or represents a C₁-C₆-alkyl or C₃-C₇-cycloalkyl radical.

A particularly preferred sub-group is formed by compounds of the general formula (I-1), in which
X represents chlorine or represents a methyl radical, and
W and Y independently of one another represent hydrogen or represent a methyl radical,
V¹ represents hydrogen, chlorine or fluorine or represents a methyl or a trifluoromethyl radical, and
V² and V³ independently of one another represent hydrogen, chlorine or fluorine,
A represents hydrogen or represents a C₁-C₄-alkyl, methoxy-C₁-C₂-alkyl or C₃-C₆-cycloalkyl radical,
B represents hydrogen or represents a methyl radical, or
A and B together with the carbon atom to which they are attached form a saturated cycle T² which optionally contains one oxygen atom and has 3 to 8 ring atoms and whose ring-forming atoms may be mono- or disubstituted by identical or different substituents selected from the group consisting of the radicals R¹, R² and R³, where R¹, R² and R³ independently of one another
   a) represent a C₁-C₃-alkyl, C₁-C₄-alkoxy, methoxy-C₁-C₃-alkyl, C₁-C₂-alkoxy ethoxy, 2,2,2-trifluoroethoxy radical and/or
   b₁) two of the radicals R¹, R² and R³ together with the ring atom of the cycle T² to which they are attached may form a further saturated cycle T³ which optionally contains one or two oxygen atoms and has 5 or 6 ring atoms and may be mono- or disubstituted by a methyl radical, or
   b₁) two of the radicals R¹, R² and R³ together with the adjacent ring atoms of the cycle T² to which they are attached may form a further aromatic cycle T³ which has 6 ring atoms, and
D represents hydrogen or represents a C₁-C₄-alkyl or C₃-C₆-cycloalkyl radical.

A particularly preferred sub-group is formed by compounds of the general formula (I-1) in which
X represents chlorine or represents a methyl radical, and
W and Y independently of one another represent hydrogen or represent a methyl radical,
V¹ represents chlorine, fluorine or a methyl radical, and
V² and V³ independently of one another represent hydrogen, chlorine or fluorine,
A and B together with the carbon atom to which they are attached form a saturated cycle T² which optionally contains one oxygen atom and has 5 to 6 ring atoms and whose ring-forming atoms may be mono- or disubstituted by identical or different substituents selected from the group consisting of the radicals R¹, R² and R³,
   where R¹, R² and R³ independently of one another
   a) represent a C₁-C₃-alkyl, C₁-C₄-alkoxy, methoxy-C₁-C₂-alkyl, 2,2,2-trifluoroethoxy radical and/or
   b) two of the radicals R¹, R² and R³ together with the ring atom of the cycle T² to which they are attached may form a further saturated cycle T³ which optionally contains one or two oxygen atoms and has 5 or 6 ring atoms and may be mono- or disubstituted by a methyl radical, and
D represents hydrogen.

A particularly preferred sub-group is formed by compounds of the general formula (I-1), in which
X represents chlorine or represents a methyl radical, and
W and Y independently of one another represent hydrogen or represent a methyl radical,
V¹ represents chlorine, fluorine or a methyl radical, and
V² and V³ independently of one another represent hydrogen, chlorine or fluorine,
A and B together with the carbon atom to which they are attached form a saturated cycle T² which optionally contains one oxygen atom and has 6 ring atoms and whose ring-forming atoms may be mono- or disubstituted by identical or different substituents selected from the group consisting of the radicals R¹, R² and R³, where R¹, R² and R³ independently of one another
   represent a C₁-C₃-alkyl, C₁-C₄-alkoxy, methoxy-C₁-C₂-alkyl, 2,2,2-trifluoroethoxy radical, and
D represents hydrogen.

Extraordinary preference is also given to the following compounds of the formula I-1:
3-(4′-chloro-2,4-dimethylbiphenyl-3-yl)-4-hydroxy-8-oxa-1-azaspiro[4.5]dec-3-en-2-one (Ex. 1-1),
(5s,8s)-3-(4′-chloro-4-methylbiphenyl-3-yl)-4-hydroxy-8-methoxy-1-azaspiro[4.5]dec-3-en-2-one (Ex. 1-2),
3-(4′-chloro-4-methylbiphenyl-3-yl)-4-hydroxy-7-methyl-8-oxa-1-azaspiro[4.5]dec-3-en-2-one (Ex. 1-3),
3-(4′-chloro-2,4-dimethylbiphenyl-3-yl)-4-hydroxy-8-methyl-1-azaspiro[4.5]dec-3-en-2-one (Ex. 1-4),
3-(4′-chloro-4-methylbiphenyl-3-yl)-4-hydroxy-7-oxa-1-azaspiro[4.5]dec-3-en-2-one (Ex. 1-5),
3-(4′-chloro-4-methylbiphenyl-3-yl)-4-hydroxy-8-oxa-1-azaspiro[4.5]dec-3-en-2-one (Ex. 1-6),
3-(4′-chloro-4,6-dimethylbiphenyl-3-yl)-4-hydroxy-8-methoxy-1-azaspiro[4.5]dec-3-en-2-one (Ex. 1-7),
3-(4′-chloro-4,6-dimethylbiphenyl-3-yl)-4-hydroxy-7-oxa-1-azaspiro[4.5]dec-3-en-2-one (Ex. 1-8),
3-(4′-chloro-2,4,6-trimethylbiphenyl-3-yl)-4-hydroxy-8-methoxy-1-azaspiro[4.5]dec-3-en-2-one (Ex. 1-9),
3-(4′-chloro-2,4-dimethylbiphenyl-3-yl)-4-hydroxy-7-oxa-1-azaspiro[4.5]dec-3-en-2-one (Ex. 1-10),
3-(4,4′-dichlorobiphenyl-3-yl)-4-hydroxy-8-methoxy-1-azaspiro[4.5]dec-3-en-2-one (Ex. 1-11),
4-hydroxy-3-[4-methyl-4′-(trifluoromethyl)biphenyl-3-yl]-1-azaspiro[4.5]dec-3-en-2-one (Ex. 1-12),
3-(4,4′-dimethylbiphenyl-3-yl)-4-hydroxy-1-azaspiro[4.5]dec-3-en-2-one (Ex. 1-13),
3-(4,4′-dimethylbiphenyl-3-yl)-4-hydroxy-7-oxa-1-azaspiro[4.5]dec-3-en-2-one (Ex. 1-14),
3-(4,4′-dimethylbiphenyl-3-yl)-4-hydroxy-8-oxa-1-azaspiro[4.5]dec-3-en-2-one (Ex. 1-15),
3-(4′-chloro-4-methylbiphenyl-3-yl)-4-hydroxy-1-methyl-1,5-dihydro-2H-pyrrol-2-one (Ex. 1-16),
3-(4′-chloro-4-methylbiphenyl-3-yl)-4-hydroxy-1-(propan-2-yl)-1,5-dihydro-2H-pyrrol-2-one (Ex. 1-17),
3-(4′-chloro-4-methylbiphenyl-3-yl)-1-cyclopropyl-4-hydroxy-1,5-dihydro-2H-pyrrol-2-one (Ex. 1-18),
3-(4′-chloro-4-methylbiphenyl-3-yl)-4-hydroxy-5-methyl-1-(propan-2-yl)-1,5-dihydro-2H-pyrrol-2-one (Ex. 1-19),
3-(4′-chloro-4-methylbiphenyl-3-yl)-4-hydroxy-8,8-dimethyl-1-azaspiro[4.5]dec-3-en-2-one (Ex. 1-20),
7-(4′-chloro-4-methylbiphenyl-3-yl)-8-hydroxy-1,3,4,8a-tetrahydro-6H-pyrrolo[2,1-c][1,4]thiazin-6-one (Ex. 1-21),
3-(4′-chloro-4-methylbiphenyl-3-yl)-1-cyclohexyl-4-hydroxy-1,5-dihydro-2H-pyrrol-2-one (Ex. 1-22),
3-(4′-chloro-4-methylbiphenyl-3-yl)-4-hydroxy-1-azaspiro[4.7]dodec-3-en-2-one (Ex. 1-23),
3-(4′-chloro-4-methylbiphenyl-3-yl)-4-hydroxy-8-(propan-2-yl)-1-azaspiro[4.5]dec-3-en-2-one (Ex. 1-24),
4′-(4′-chloro-4-methylbiphenyl-3-yl)-3′-hydroxy-1,3-dihydrospiro[indene-2,2′-pyrrol]-5′(1′H)-one (Ex. 1-25),
3-(4′-chloro-4-methylbiphenyl-3-yl)-4-hydroxy-1-azaspiro[4.6]undec-3-en-2-one (Ex. 1-26),
3-(4′-chloro-4-methylbiphenyl-3-yl)-4-hydroxy-1-(2-methylpropyl)-1,5-dihydro-2H-pyrrol-2-one (Ex. 1-27),
3-(4′-chloro-4-methylbiphenyl-3-yl)-8-ethoxy-4-hydroxy-1-azaspiro[4.5]dec-3-en-2-one (Ex. 1-28),
(5s,8s)-3-(4,4′-dichlorobiphenyl-3-yl)-4-hydroxy-8-(2,2,2-trifluoroethoxy)-1-azaspiro[4.5]dec-3-en-2-one (Ex. 1-29),
(5s,8s)-3-(4′-chloro-2,4-dimethylbiphenyl-3-yl)-4-hydroxy-8-(2,2,2-trifluoroethoxy)-1-azaspiro[4.5]dec-3-en-2-one (Ex. 1-30),
3-(4′-fluoro-2,4-dimethylbiphenyl-3-yl)-4-hydroxy-8-(2,2,2-trifluoroethoxy)-1-azaspiro[4.5]dec-3-en-2-one (Ex. 1-31),
3-(4′-fluoro-4-methylbiphenyl-3-yl)-4-hydroxy-8-(2,2,2-trifluoroethoxy)-1-azaspiro[4.5]dec-3-en-2-one (Ex. 1-32),
3-(4′-chloro-4-methylbiphenyl-3-yl)-4-hydroxy-8-(2,2,2-trifluoroethoxy)-1-azaspiro[4.5]dec-3-en-2-one (Ex. 1-33),
11-(4′-chloro-2,4-dimethylbiphenyl-3-yl)-12-hydroxy-1-oxa-9-azadispiro[4.2.4.2]tetradec-11-en-10-one (Ex. 1-34),
11-(3′,4′-difluoro-4-methylbiphenyl-3-yl)-12-hydroxy-1-oxa-9-azadispiro[4.2.4.2]tetradec-11-en-10-one (Ex. 1-35),
4-hydroxy-3-(3′,4′,5′-trifluoro-4-methylbiphenyl-3-yl)-8-oxa-1-azaspiro[4.5]dec-3-en-2-one (Ex. 1-36),
3-(3′,4′-difluoro-4-methylbiphenyl-3-yl)-4-hydroxy-8-oxa-1-azaspiro[4.5]dec-3-en-2-one (Ex. 1-37),
3-(4-chloro-3′,4′,5′-trifluorobiphenyl-3-yl)-4-hydroxy-8-oxa-1-azaspiro[4.5]dec-3-en-2-one (Ex. 1-38),
3-(4-chloro-3′-fluoro-4′-methylbiphenyl-3-yl)-4-hydroxy-8-oxa-1-azaspiro[4.5]dec-3-en-2-one (Ex. 1-39),
3-(4′-fluoro-2,4-dimethylbiphenyl-3-yl)-4-hydroxy-1-azaspiro[4.5]dec-3-en-2-one (Ex. 1-40),
3-(4-chloro-4′-fluorobiphenyl-3-yl)-4-hydroxy-8-oxa-1-azaspiro[4.5]dec-3-en-2-one (Ex. 1-41),
(5s,8s)-3-(4′-fluoro-2,4,6-trimethylbiphenyl-3-yl)-4-hydroxy-8-methoxy-1-azaspiro[4.5]dec-3-en-2-one (Ex. 1-42),
(5s,8s)-3-(3′-chloro-4′-fluoro-4-methylbiphenyl-3-yl)-4-hydroxy-8-methoxy-1-azaspiro[4.5]dec-3-en-2-one (Ex. 1-43),
(5s,8s)-3-(4-chloro-4′-fluorobiphenyl-3-yl)-4-hydroxy-8-methoxy-1-azaspiro[4.5]dec-3-en-2-one (Ex. 1-44),
(5s,8s)-3-(4′-fluoro-2,4-dimethylbiphenyl-3-yl)-4-hydroxy-8-methoxy-1-azaspiro[4.5]dec-3-en-2-one (Ex. 1-45),
3-(4′-chloro-4-methylbiphenyl-3-yl)-4-hydroxy-5-(methoxymethyl)-5-methyl-1,5-dihydro-2H-pyrrol-2-one (Ex. 1-46),
3-(4′-chloro-4-methylbiphenyl-3-yl)-4-hydroxy-5-(2-methoxyethyl)-5-methyl-1,5-dihydro-2H-pyrrol-2-one (Ex. 1-47),
3-(4′-chloro-2,4-dimethylbiphenyl-3-yl)-4-hydroxy-7-methoxy-1-azaspiro[4.4]non-3-en-2-one (Ex. 1-48),
rel-(5R,7R)-3-(4′-chloro-4-methylbiphenyl-3-yl)-4-hydroxy-7-(2-methylpropoxy)-1-azaspiro[4.4]non-3-en-2-one (Ex. 1-49),
rel-(5R,7S)-3-(4′-chloro-4-methylbiphenyl-3-yl)-7-(2-ethoxyethoxy)-4-hydroxy-1-azaspiro[4.4]non-3-en-2-one (Ex. 1-50),
3-(4′-chloro-4-methylbiphenyl-3-yl)-4-hydroxy-7-(methoxymethyl)-1-azaspiro[4.5]dec-3-en-2-one (Ex. 1-51),
3-(4,4′-dichlorobiphenyl-3-yl)-4-hydroxy-7-(methoxymethyl)-1-azaspiro[4.5]dec-3-en-2-one (Ex. 1-52),
3-(4′-chloro-2,4-dimethylbiphenyl-3-yl)-4-hydroxy-8-(methoxymethyl)-1-azaspiro[4.5]dec-3-en-2-one (Ex. 1-53),
3-(4,4′-dichlorobiphenyl-3-yl)-4-hydroxy-8-(methoxymethyl)-1-azaspiro[4.5]dec-3-en-2-one (Ex. 1-54),
3-(4′-chloro-2,4-dimethylbiphenyl-3-yl)-4-hydroxy-7-(2-methoxyethyl)-1-azaspiro[4.5]dec-3-en-2-one (Ex. 1-55),
3-(4′-chloro-4-methylbiphenyl-3-yl)-4-hydroxy-8-(methoxymethyl)-1-azaspiro[4.5]dec-3-en-2-one (Ex. 1-56),
3-(4′-chloro-2,4-dimethylbiphenyl-3-yl)-4-hydroxy-8-(2-methoxyethyl)-1-azaspiro[4.5]dec-3-en-2-one (Ex. 1-57),
3-(4,4′-dichlorobiphenyl-3-yl)-4-hydroxy-9,13-dioxa-1-azadispiro[4.2.5.2]pentadec-3-en-2-one (Ex. 1-58),
3-(4′-chloro-4-methylbiphenyl-3-yl)-4-hydroxy-9,13-dioxa-1-azadispiro[4.2.5.2]pentadec-3-en-2-one (Ex. 1-59),
11-(4′-chloro-4-methylbiphenyl-3-yl)-12-hydroxy-1,4-dioxa-9-azadispiro[4.2.4.2]tetradec-11-en-10-one (Ex. 1-60),
11-(4,4′-dichlorobiphenyl-3-yl)-12-hydroxy-1,4-dioxa-9-azadispiro[4.2.4.2]tetradec-11-en-10-one (Ex. 1-61),
11-(4′-chloro-2,4-dimethylbiphenyl-3-yl)-12-hydroxy-2-methyl-1,4-dioxa-9-azadispiro[4.2.4.2]tetradec-11-en-10-one (Ex. 1-62),
11-(4′-chloro-4-methylbiphenyl-3-yl)-12-hydroxy-2-methyl-1,4-dioxa-9-azadispiro[4.2.4.2]tetradec-11-en-10-one (Ex. 1-63),
11-(4′-chloro-4-methylbiphenyl-3-yl)-12-hydroxy-2,3-dimethyl-1,4-dioxa-9-azadispiro[4.2.4.2]tetradec-11-en-10-one (Ex. 1-64),
3-(4,4′-dichlorobiphenyl-3-yl)-4-hydroxy-11,11-dimethyl-9,13-dioxa-1-azadispiro[4.2.5.2]pentadec-3-en-2-one (Ex. 1-65),
3-(4′-chloro-4-methylbiphenyl-3-yl)-4-hydroxy-1′-methyl-9,13-dioxa-1-azadispiro[4.2.5.2]pentadec-3-en-2-one (Ex. 1-66),
3-(4′-chloro-4-methylbiphenyl-3-yl)-4-hydroxy-11,11-dimethyl-9,13-dioxa-1-azadispiro[4.2.5.2]pentadec-3-en-2-one (Ex. 1-67),
11-(4,4′-dichlorobiphenyl-3-yl)-12-hydroxy-2-methyl-1,4-dioxa-9-azadispiro[4.2.4.2]tetradec-11-en-10-one (Ex. 1-68),
3-(4,4′-dichlorobiphenyl-3-yl)-4-hydroxy-1′-methyl-9,13-dioxa-1-azadispiro[4.2.5.2]pentadec-3-en-2-one (Ex. 1-69),
11-(4′-chloro-2,4-dimethylbiphenyl-3-yl)-12-hydroxy-1,4-dioxa-9-azadispiro[4.2.4.2]tetradec-11-en-10-one (Ex. 1-70),
11-(4,4′-dichlorobiphenyl-3-yl)-12-hydroxy-2,3-dimethyl-1,4-dioxa-9-azadispiro[4.2.4.2]tetradec-11-en-10-one (Ex. 1-71),
3-(4′-chloro-2,4-dimethylbiphenyl-3-yl)-4-hydroxy-7-methoxy-1-azaspiro[4.5]dec-3-en-2-one (Ex. 1-72),
3-(4′-chloro-2,4-dimethylbiphenyl-3-yl)-7-ethoxy-4-hydroxy-1-azaspiro[4.5]dec-3-en-2-one (Ex. 1-73),
rel-(5R,7R)-3-(4,4′-dichlorobiphenyl-3-yl)-4-hydroxy-7-(2-methoxyethoxy)-1-azaspiro[4.5]dec-3-en-2-one (Ex. 1-74),
3-(4′-chloro-2,4,6-trimethylbiphenyl-3-yl)-8-ethoxy-4-hydroxy-1-azaspiro[4.5]dec-3-en-2-one (Ex. 1-75),
3-(4′-chloro-4-methylbiphenyl-3-yl)-4-hydroxy-8-methyl-1-azaspiro[4.5]dec-3-en-2-one (Ex. 1-76),
3-(4′-chloro-4-methylbiphenyl-3-yl)-4-hydroxy-1-azaspiro[4.5]dec-3-en-2-one (Ex. 1-77),
3-(4′-chloro-4-methylbiphenyl-3-yl)-4-hydroxy-5-methyl-5-(propan-2-yl)-1,5-dihydro-2H-pyrrol-2-one (Ex. 1-78),
3-(3′-chloro-4-methylbiphenyl-3-yl)-8-ethoxy-4-hydroxy-1-azaspiro[4.5]dec-3-en-2-one (Ex. 1-79),
3-(2′,5′-difluoro-4-methylbiphenyl-3-yl)-4-hydroxy-8-methoxy-1-azaspiro[4.5]dec-3-en-2-one (Ex. 1-80),
3-(3′,4′-dichloro-4-methylbiphenyl-3-yl)-4-hydroxy-8-methoxy-1-azaspiro[4.5]dec-3-en-2-one (Ex. 1-81),
3-(4′-chloro-4-methylbiphenyl-3-yl)-4-hydroxy-7-methyl-1-azaspiro[4.5]dec-3-en-2-one (Ex. 1-82),
3-(4′-chloro-4-methylbiphenyl-3-yl)-4-hydroxy-8-propyl-1-azaspiro[4.5]dec-3-en-2-one (Ex. 1-83),
6-(4′-chloro-4-methylbiphenyl-3-yl)-7-hydroxy-4-azaspiro[2.4]hept-6-en-5-one (Ex. 1-84),
3-(4′-chloro-4-methylbiphenyl-3-yl)-4-hydroxy-1-azaspiro[4.4]non-3-en-2-one (Ex. 1-85),
3-(4′-chloro-4-methylbiphenyl-3-yl)-5-cyclopropyl-4-hydroxy-5-methyl-1,5-dihydro-2H-pyrrol-2-one (Ex. 1-86),
(5r,8r)-3-(4′-chloro-2,4-dimethylbiphenyl-3-yl)-4-hydroxy-8-methoxy-8-methyl-1-azaspiro[4.5]dec-3-en-2-one (Ex. 1-87),
(5r,8r)-3-(4′-chloro-2,4-dimethylbiphenyl-3-yl)-8-ethyl-4-hydroxy-8-methoxy-1-azaspiro[4.5]dec-3-en-2-one (Ex. 1-88),
(5r,8r)-8-ethyl-3-(4′-fluoro-2,4-dimethylbiphenyl-3-yl)-4-hydroxy-8-methoxy-1-azaspiro[4.5]dec-3-en-2-one (Ex. 1-89).

A preferred sub-group is also formed by compounds of the general formula (I-2),

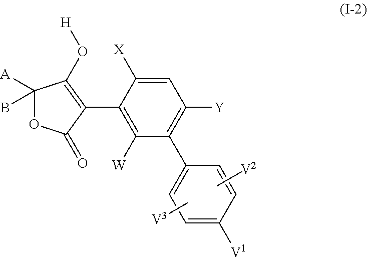

in which
X represents halogen or
   represents an optionally monohalogen- or polyhalogen-substituted C₁-C₃-alkyl or C₁-C₃-alkoxy radical, and
W and Y independently of one another represent hydrogen or halogen or represent an optionally monohalogen- or polyhalogen-substituted C₁-C₃-alkyl radical, and
V¹, V² and V³ independently of one another represent hydrogen or halogen or represent a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
A represents hydrogen or
   represents an optionally monohalogen- or polyhalogen-substituted C₁-C₆-alkyl or C₁-C₆-alkoxy-C₁-C₆-alkyl radical or
   represents a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, and
B represents hydrogen or represents a C₁-C₆-alkyl or C₁-C₃-alkoxy-C₁-C₃-alkyl radical, or
A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen or hydroxyl or
   b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
   c) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of R⁴, R⁵ and R⁶,
      where R⁴, R⁵ and R⁶ independently of one another represent a C₁-C₃-alkyl or C₁-C₃-alkoxy radical.

In formula (I-2), X may represent:

halogen or an optionally monohalogen- or polyhalogen-substituted C₁-C₃-alkyl or C₁-C₃-alkoxy radical.

In formula (I-2), X may preferably represent:

chlorine or a methyl radical.

In formula (I-2), W and Y may independently of one another represent:

hydrogen or halogen or an optionally monohalogen- or polyhalogen-substituted C₁-C₃-alkyl radical.

In formula (I-2), W and Y independently of one another may preferably represent:

hydrogen or a methyl radical.

In formula (I-2), X, W and Y independently of one another may with extraordinary preference represent:

X represents methyl and W and Y represent hydrogen or
X and W represent methyl and Y represents hydrogen or
X and Y represent methyl and W represents hydrogen.

In formula (I-2), V¹, V² and V³ independently of one another may represent:

hydrogen or halogen or
a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical.

In formula (I-2), V¹, V² and V³ independently of one another may preferably represent:

hydrogen, chlorine or fluorine or a methyl or a trifluoromethyl radical.

In formula (I-2), V¹, V² and V³ may more preferably represent:

V¹ represents hydrogen, chlorine or fluorine or represents a methyl or a trifluoromethyl radical, and
V² and V³ independently of one another represent hydrogen, chlorine or fluorine.

In formula (I-2), V¹, V² and V³ independently of one another may particularly preferably represent:

hydrogen, chlorine or fluorine.

In formula (I-2), V¹ may with extraordinary preference represent chlorine or fluorine, in particular chlorine.

In formula (I-2), A may represent:

hydrogen or
an optionally monohalogen- or polyhalogen-substituted C₁-C₆-alkyl or C₁-C₆-alkoxy-C₁-C₆-alkyl radical or a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical.

In formula (I-2), A may preferably represent:

hydrogen or
an optionally halogen-substituted C₁-C₄-alkyl or C₁-C₃-alkoxy-C₁-C₃-alkyl radical or a C₃-C₆-cycloalkyl radical which may be mono- or polysubstituted by identical or different substituents selected from the group consisting of halogen and a C₁-C₃-alkyl radical.

In formula (I-2), A may more preferably represent:

hydrogen or
an optionally halogen-substituted C₁-C₄-alkyl or C₁-C₃-alkoxy-C₁-C₃-alkyl radical or a C₃-C₆-cycloalkyl radical.

In formula (I-2), A may particularly preferably represent:

hydrogen or
a C₁-C₄-alkyl, methoxy-C₁-C₂-alkyl or a C₃-C₆-cycloalkyl radical.

In formula (I-2), B may represent:

hydrogen or a C₁-C₆-alkyl or C₁-C₃-alkoxy-C₁-C₃-alkyl radical.

In formula (I-2), B may preferably represent:

hydrogen or a C₁-C₃-alkyl radical.

In formula (I-2), B may more preferably represent:

hydrogen or a methyl radical.

In formula (I-2), A and B together with the carbon atom to which they are attached may form:

a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
c) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of the radicals R⁴, R⁵ and R⁶,
   where R⁴, R⁵ and R⁶ independently of one another represent a C₁-C₃-alkyl or C₁-C₃-alkoxy radical.

In formula (I-2), A and B together with the carbon atom to which they are attached may preferably form:

a saturated cycle T² which optionally contains one oxygen atom and has 3 to 8 ring atoms and whose ring-forming atoms may be mono- or disubstituted by identical or different substituents selected from the group consisting of the radicals R¹, R² and R³, where R¹, R² and R³ independently of one another
a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
b₁) two of the radicals R¹, R² and R³ together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
b₂) two of the radicals R¹, R² and R³ together with the adjacent ring atoms of the cycle T² to which they are attached may form a further aromatic cycle T³ which has 6 ring atoms and may be mono- or disubstituted by a C₁-C₃-alkyl radical.

In formula (I-2), A and B together with the carbon atom to which they are attached may more preferably form:

a saturated cycle T² which optionally contains one oxygen or sulphur atom and has 5 to 7 ring atoms and whose ring-forming atoms may be mono- or disubstituted by identical or different substituents selected from the group consisting of the radicals R¹, R² and R³,
where R¹, R² and R³ independently of one another
a) represent a C₁-C₃-alkyl, C₁-C₃-alkoxy, C₁-C₃-alkoxy-C₁-C₃-alkyl or halo-C₁-C₃-alkoxy radical and/or
b) two of the radicals R¹, R² and R³ together with the ring atom of the cycle T² to which they are attached may form a further saturated cycle T³ which optionally contains one or two oxygen atoms and has 5 or 6 ring atoms.

In formula (I-2), A and B together with the carbon atom to which they are attached may particularly preferably form:

a saturated cycle T² which optionally contains one oxygen atom and has 5 to 6 ring atoms and whose ring-forming atoms may be mono- or disubstituted by identical or different substituents selected from the group consisting of the radicals R¹, R² and R³, where R¹, R² and R³ independently of one another
a) represent a C₁-C₃-alkyl, C₁-C₄-alkoxy, methoxy-C₁-C₂-alkyl, 2,2,2-trifluoroethoxy radical and/or
b) two of the radicals R¹, R² and R³ together with the ring atom of the cycle T² to which they are attached may form a further saturated cycle T³ which optionally contains one or two oxygen atoms and has 5 or 6 ring atoms and may be mono- or disubstituted by a methyl radical.

In formula (I-2), A and B together with the carbon atom to which they are attached may with extraordinary preference form:

a saturated cycle T² which optionally contains one oxygen atom and has 6 ring atoms and whose ring-forming atoms may be mono- or disubstituted by identical or different substituents selected from the group consisting of the radicals R¹, R² and R³, where R¹, R² and R³ independently of one another
represent a C₁-C₃-alkyl, C₁-C₄-alkoxy, methoxy-C₁-C₂-alkyl, 2,2,2-trifluoroethoxy radical.

In formula (I-2), A and B together with the carbon atom to which they are attached may very preferably form a cyclohexane ring or tetrahydropyran ring.

Any substituents R¹, R² and R³ of the cycle T² formed by A and B very preferably independently of one another represent a C₁-C₃-alkyl, C₁-C₂-alkoxy, methoxy-C₁-C₂-alkyl or 2,2,2-trifluoroethoxy radical.

Particular preference is given to only one substituent, where R¹ represents a C₁-C₃-alkyl, C₁-C₂-alkoxy, methoxy-C₁-C₂-alkyl or 2,2,2-trifluoroethoxy radical.

A particularly preferred sub-group is formed by compounds of the general formula (I-2), in which
X represents chlorine or represents a methyl radical, and
W and Y independently of one another represent hydrogen or represent a methyl radical,
V¹, V² and V³ independently of one another represent hydrogen, chlorine or fluorine or represent a methyl or a trifluoromethyl radical, and
A represents hydrogen or represents an optionally halogen-substituted C₁-C₃-alkyl or C₁-C₃-alkoxy-C₁-C₃-alkyl radical or
   represents a C₃-C₆-cycloalkyl radical which may be mono- or polysubstituted by identical or different substituents selected from the group consisting of halogen and a C₁-C₃-alkyl radical, and
B represents hydrogen or represents a C₁-C₃-alkyl radical, or
A and B together with the carbon atom to which they are attached form a saturated cycle T² which optionally contains one oxygen atom and has 3 to 8 ring atoms and whose ring-forming atoms may be mono- or disubstituted by identical or different substituents selected from the group consisting of the radicals R¹, R² and R³, where R¹, R² and R³ independently of one another
   a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
   b₁) two of the radicals R¹, R² and R³ together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
   b₂) two of the radicals R¹, R² and R³ together with the adjacent ring atoms of the cycle T² to which they are attached may form a further aromatic cycle T³ which has 6 ring atoms and may be mono- or disubstituted by a C₁-C₃-alkyl radical.

A particularly preferred sub-group within the group of the compounds of the formula (I-2) is formed by compounds of the formula (I-2) in which
X represents chlorine or represents a methyl radical, and
W and Y independently of one another represent hydrogen or represent a methyl radical,
V¹ represents hydrogen, chlorine or fluorine, and
V² and V³ independently of one another represent hydrogen, chlorine or fluorine,
A represents hydrogen or represents a C₁-C₃-alkyl or C₃-C₆-cycloalkyl radical, and
B represents hydrogen or represents a methyl radical, or
A and B together with the carbon atom to which they are attached form a saturated cycle T² which optionally contains one oxygen or sulphur atom and has 5 to 7 ring atoms and whose ring-forming atoms may be mono- or disubstituted by identical or different substituents selected from the group consisting of the radicals R¹, R² and R³,
   where R¹, R² and R³ independently of one another
   a) represent a C₁-C₃-alkyl, C₁-C₃-alkoxy, C₁-C₃-alkoxy-C₁-C₃-alkyl or halo-C₁-C₃-alkoxy radical and/or
   b) two of the radicals R¹, R² and R³ together with the ring atom of the cycle T² to which they are attached may form a further saturated cycle T³ which optionally contains one or two oxygen atoms and has 5 or 6 ring atoms.

Extraordinary preference is given to the following compounds of the formula I-2:
3-(4′-chloro-4-methylbiphenyl-3-yl)-8-ethyl-4-hydroxy-1-oxaspiro[4.5]dec-3-en-2-one (Ex. 2-1),
3-(4′-chloro-2,4-dimethylbiphenyl-3-yl)-4-hydroxy-1-oxaspiro[4.4]non-3-en-2-one (Ex. 2-2),
3-(4′-chloro-4-methylbiphenyl-3-yl)-4-hydroxy-5,5-dimethylfuran-2(5H)-one (Ex. 2-3),
3-(4′-chloro-2,4-dimethylbiphenyl-3-yl)-4-hydroxy-1-oxaspiro[4.5]dec-3-en-2-one (Ex. 2-4),
3-(4′-chloro-2,4,6-trimethylbiphenyl-3-yl)-4-hydroxy-5,5-dimethylfuran-2(5H)-one (Ex. 2-5),
3-(4′-chloro-2,4,6-trimethylbiphenyl-3-yl)-4-hydroxy-1-oxaspiro[4.5]dec-3-en-2-one (Ex. 2-6),
3-(2′-chloro-4-methylbiphenyl-3-yl)-4-hydroxy-8-methoxy-1-oxaspiro[4.5]dec-3-en-2-one (Ex. 2-7),
6-(4′-chloro-4-methylbiphenyl-3-yl)-7-hydroxy-4-oxaspiro[2.4]hept-6-en-5-one (Ex. 2-8),
3-(4′-chloro-4-methylbiphenyl-3-yl)-4-hydroxy-1,7-dioxaspiro[4.5]dec-3-en-2-one (Ex. 2-9),
3-(4′-chloro-4-methylbiphenyl-3-yl)-4-hydroxy-1-oxaspiro[4.5]dec-3-en-2-one (Ex. 2-10),
3-(4′-chloro-4,6-dimethylbiphenyl-3-yl)-4-hydroxy-7-methoxy-1-oxaspiro[4.5]dec-3-en-2-one (Ex. 2-11),
3-(4′-chloro-2,4,6-trimethylbiphenyl-3-yl)-4-hydroxy-7-methoxy-1-oxaspiro[4.5]dec-3-en-2-one (Ex. 2-12),
3-(4,4′-dichlorobiphenyl-3-yl)-4-hydroxy-7-methoxy-1-oxaspiro[4.5]dec-3-en-2-one (Ex. 2-13),
3-(4′-chloro-4-methylbiphenyl-3-yl)-4-hydroxy-7-methoxy-1-oxaspiro[4.5]dec-3-en-2-one (Ex. 2-14),
11-(4′-chloro-4-methylbiphenyl-3-yl)-12-hydroxy-1,4,9-trioxadispiro[4.2.4.2]tetradec-11-en-10-one (Ex. 2-15),
3-(4,4′-dichlorobiphenyl-3-yl)-4-hydroxy-8-(methoxymethyl)-1-oxaspiro[4.5]dec-3-en-2-one (Ex. 2-16),
3-(4′-chloro-2,4-dimethylbiphenyl-3-yl)-4-hydroxy-8-(methoxymethyl)-1-oxaspiro[4.5]dec-3-en-2-one (Ex. 2-17),
3-(4′-chloro-4-methylbiphenyl-3-yl)-4-hydroxy-7-(2-methoxyethyl)-1-oxaspiro[4.5]dec-3-en-2-one (Ex. 2-18),
3-(4,4′-dichlorobiphenyl-3-yl)-4-hydroxy-7-(2-methoxyethyl)-1-oxaspiro[4.5]dec-3-en-2-one (Ex. 2-19),
3-(4′-chloro-4-methylbiphenyl-3-yl)-4-hydroxy-8-(methoxymethyl)-1-oxaspiro[4.5]dec-3-en-2-one (Ex. 2-20),
3-(4′-fluoro-4-methylbiphenyl-3-yl)-4-hydroxy-1-oxaspiro[4.5]dec-3-en-2-one (Ex. 2-21),
3-(3′,4′-difluoro-4-methylbiphenyl-3-yl)-4-hydroxy-1-oxaspiro[4.4]non-3-en-2-one (Ex. 2-22),
3-(3′-chloro-4′-fluoro-4-methylbiphenyl-3-yl)-4-hydroxy-1-oxaspiro[4.4]non-3-en-2-one (Ex. 2-23),
3-(4-chloro-3′,4′,5′-trifluorobiphenyl-3-yl)-4-hydroxy-1-oxaspiro[4.4]non-3-en-2-one (Ex. 2-24),
3-(4-chloro-3′,4′-difluorobiphenyl-3-yl)-4-hydroxy-1-oxaspiro[4.5]dec-3-en-2-one (Ex. 2-25),
(5s,8r)-3-(4′-chloro-2,4-dimethylbiphenyl-3-yl)-4-hydroxy-1,9-dioxadispiro[4.2.4.2]tetradec-3-en-2-one (Ex. 2-26),
3-(4,4′-dichlorobiphenyl-3-yl)-4-hydroxy-1,9-dioxadispiro[4.2.4.2]tetradec-3-en-2-one (Ex. 2-27),
(5r,8s)-3-(4′-fluoro-4-methylbiphenyl-3-yl)-4-hydroxy-1,9-dioxadispiro[4.2.4.2]tetradec-3-en-2-one (Ex. 2-28),
3-(4′-chloro-4-methylbiphenyl-3-yl)-4-hydroxy-1,9-dioxadispiro[4.2.4.2]tetradec-3-en-2-one (Ex. 2-29),
(5r,8s)-3-(4′-chloro-4,6-dimethylbiphenyl-3-yl)-4-hydroxy-1,9-dioxadispiro[4.2.4.2]tetradec-3-en-2-one (Ex. 2-30),
(5r,8s)-3-(4′-chloro-2,4,6-trimethylbiphenyl-3-yl)-4-hydroxy-1,9-dioxadispiro[4.2.4.2]tetradec-3-en-2-one (Ex. 2-31),
3-(4′-chloro-2,4-dimethylbiphenyl-3-yl)-4-hydroxy-8-(2,2,2-trifluoroethoxy)-1-oxaspiro[4.5]dec-3-en-2-one (Ex. 2-32),
3-(4′-chloro-4-methylbiphenyl-3-yl)-4-hydroxy-8-(2,2,2-trifluoroethoxy)-1-oxaspiro[4.5]dec-3-en-2-one (Ex. 2-33),
3-(4′-chloro-2,4-dimethylbiphenyl-3-yl)-4-hydroxy-8-methoxy-1-oxaspiro[4.5]dec-3-en-2-one (Ex. 2-34),
5-tert-butyl-3-(4′-chloro-4-methylbiphenyl-3-yl)-4-hydroxyfuran-2(5H)-one (Ex. 2-35),
3-(4′-chloro-4-methylbiphenyl-3-yl)-4-hydroxy-8-propyl-1-oxaspiro[4.5]dec-3-en-2-one (Ex. 2-36),
3-(4′-chloro-4-methylbiphenyl-3-yl)-4-hydroxy-1,8-dioxaspiro[4.5]dec-3-en-2-one (Ex. 2-37),
3-(4′-chloro-4-methylbiphenyl-3-yl)-4-hydroxy-1-oxaspiro[4.6]undec-3-en-2-one (Ex. 2-38),
3-(4′-chloro-4-methylbiphenyl-3-yl)-4-hydroxy-8-methoxy-1-oxaspiro[4.5]dec-3-en-2-one (Ex. 2-39),
3-(4′-chloro-4-methylbiphenyl-3-yl)-5-cyclohexyl-4-hydroxy-5-methylfuran-2(5H)-one (Ex. 2-40),
3-(4′-chloro-4,6-dimethylbiphenyl-3-yl)-4-hydroxy-1,8-dioxaspiro[4.5]dec-3-en-2-one (Ex. 2-41),
3-(4′-chloro-4,6-dimethylbiphenyl-3-yl)-4-hydroxy-1-oxa-8-thiaspiro[4.5]dec-3-en-2-one (Ex. 2-42),
3-(4′-chloro-4,6-dimethylbiphenyl-3-yl)-4-hydroxy-1-oxaspiro[4.6]undec-3-en-2-one (Ex. 2-43),
3-(4′-chloro-4,6-dimethylbiphenyl-3-yl)-4-hydroxy-8-methoxy-1-oxaspiro[4.5]dec-3-en-2-one (Ex. 2-44),
3-(4′-chloro-4,6-dimethylbiphenyl-3-yl)-4-hydroxy-5-methylfuran-2(5H)-one (Ex. 2-45),
3-(4′-chloro-4,6-dimethylbiphenyl-3-yl)-4-hydroxy-1,7-dioxaspiro[4.5]dec-3-en-2-one (Ex. 2-46),
3-(4′-chloro-4,6-dimethylbiphenyl-3-yl)-4-hydroxy-8-propoxy-1-oxaspiro[4.5]dec-3-en-2-one (Ex. 2-47),
3-(4′-chloro-2,4,6-trimethylbiphenyl-3-yl)-4-hydroxy-1-oxaspiro[4.6]undec-3-en-2-one (Ex. 2-48),
3-(4′-chloro-2,4,6-trimethylbiphenyl-3-yl)-4-hydroxy-1-oxaspiro[4.4]non-3-en-2-one (Ex. 2-49),
3-(3′-chloro-4-methylbiphenyl-3-yl)-4-hydroxy-1-oxaspiro[4.6]undec-3-en-2-one (Ex. 2-50).

Extraordinary preference is given to the following compounds of the formulae I-3, I-6, I-7, I-8, I-9, I-10 and I-11:
3-(4′-chloro-4-methylbiphenyl-3-yl)-4-hydroxy-8-methoxy-1-thiaspiro[4.5]dec-3-en-2-one (Ex. 3-1)
3-(4′-chloro-4-methylbiphenyl-3-yl)-4-hydroxy-1-thiaspiro[4.5]dec-3-en-2-one (Ex. 3-2)
3-(4′-chloro-4,6-dimethylbiphenyl-3-yl)-4-hydroxy-1-thiaspiro[4.5]dec-3-en-2-one (Ex. 3-3)
3-(4′-chloro-2,4,6-trimethylbiphenyl-3-yl)-4-hydroxy-1-thiaspiro[4.5]dec-3-en-2-one (Ex. 3-4)
3-(4,4′-dimethylbiphenyl-3-yl)-4-hydroxyspiro[4.5]dec-3-en-2-one (Ex. 6-1)
3-(2′,4′-dichloro-4-methylbiphenyl-3-yl)-4-hydroxyspiro[4.5]dec-3-en-2-one (Ex. 6-2)
3-(3′-chloro-4-methylbiphenyl-3-yl)-4-hydroxyspiro[4.5]dec-3-en-2-one (Ex. 6-3)
3-(4′-chloro-4-methylbiphenyl-3-yl)-4-hydroxyspiro[4.4]non-3-en-2-one (Ex. 6-4)
3-(4′-chloro-4-methylbiphenyl-3-yl)-8-ethyl-4-hydroxyspiro[4.5]dec-3-en-2-one (Ex. 6-5)
3-(4′-chloro-4-methylbiphenyl-3-yl)-4-hydroxy-8-propylspiro[4.5]dec-3-en-2-one (Ex. 6-6)
3-(4′-chloro-4-methylbiphenyl-3-yl)-4-hydroxyspiro[4.6]undec-3-en-2-one (Ex. 6-7)
3-(4′-chloro-4,6-dimethylbiphenyl-3-yl)-4-hydroxyspiro[4.5]dec-3-en-2-one (Ex. 6-8)
3-(4′-chloro-2,4,6-trimethylbiphenyl-3-yl)-4-hydroxyspiro[4.5]dec-3-en-2-one (Ex. 6-9)
2-(4′-chloro-4-methylbiphenyl-3-yl)-3-hydroxy-5-methylcyclohex-2-en-1-one (Ex. 7-1)
3-(4′-chloro-4-methylbiphenyl-3-yl)-4-hydroxyspiro[5.5]undec-3-en-2-one (Ex. 7-2)
2-(4′-chloro-4,6-dimethylbiphenyl-3-yl)-3-hydroxy-5,5-dimethylcyclohex-2-en-1-one (Ex. 7-3)
2-(4′-chloro-4,6-dimethylbiphenyl-3-yl)-3-hydroxy-4,4-dimethylcyclohex-2-en-1-one (Ex. 7-4)
2-(4′-chloro-4-methylbiphenyl-3-yl)-3-hydroxy-5,5-dimethylcyclohex-2-en-1-one (Ex. 7-5)
2-(4′-fluoro-2,4-dimethylbiphenyl-3-yl)tetrahydro-1H-pyrazolo[1,2-a]pyridazine-1,3(2H)-dione (Ex. 8-1)
2-(4,4′-dichlorobiphenyl-3-yl)tetrahydro-1H-pyrazolo[1,2-a]pyridazine-1,3(2H)-dione (Ex. 8-2)
2-(4′-chloro-4-methylbiphenyl-3-yl)tetrahydro-1H-pyrazolo[1,2-a]pyridazine-1,3(2H)-dione (Ex. 8-3)
2-(3′,4-dichloro-4′-fluorobiphenyl-3-yl)tetrahydro-1H-pyrazolo[1,2-a]pyridazine-1,3(2H)-dione (Ex. 8-4)
8-(2′,4′-difluoro-4-methylbiphenyl-3-yl)tetrahydro-7H-pyrazolo[1,2-d][1,4,5]oxadiazepine-7,8(8H)-dione (Ex. 8-5)
8-(4′-chloro-4-methylbiphenyl-3-yl)tetrahydro-7H-pyrazolo[1,2-d][1,4,5]oxadiazepine-7,9(8H)-dione (Ex. 8-6)
8-(4′-fluoro-2,4-dimethylbiphenyl-3-yl)tetrahydro-7H-pyrazolo[1,2-d][1,4,5]oxadiazepine-7,9(8H)-dione (Ex. 8-7)
8-(4′-chloro-2,4-dimethylbiphenyl-3-yl)tetrahydro-7H-pyrazolo[1,2-d][1,4,5]oxadiazepine-7,9(8H)-dione (Ex. 8-8)
8-(2′,4,4′-trichlorobiphenyl-3-yl)tetrahydro-7H-pyrazolo[1,2-d][1,4,5]oxadiazepine-7,9(8H)-dione (Ex. 8-9)
8-(3′,4,4′-trichlorobiphenyl-3-yl)tetrahydro-7H-pyrazolo[1,2-d][1,4,5]oxadiazepine-7,9(8H)-dione (Ex. 8-10)
8-(4-chloro-2′,4′-difluorobiphenyl-3-yl)tetrahydro-7H-pyrazolo[1,2-d][1,4,5]oxadiazepine-7,9(8H)-dione (Ex. 8-11)
2-(4′-chloro-4-methylbiphenyl-3-yl)-6-fluoro-6-methyldihydro-1H,5H-pyrazolo[1,2-a]pyrazole-1,3(2H)-dione (Ex. 8-12)
2-(4,4′-dichlorobiphenyl-3-yl)-6-fluoro-6-methyldihydro-1H,5H-pyrazolo[1,2-a]pyrazole-1,3(2H)-dione (Ex. 8-13)
4-(4′-chloro-4-methylbiphenyl-3-yl)-1,2-dimethyl-1H-pyrazole-3,5(2H,4H)-dione (Ex. 8-14)
4-(4,4′-dichlorobiphenyl-3-yl)-1,2-dimethyl-1H-pyrazole-3,5(2H,4H)-dione (Ex. 8-15)
2-(4′-chloro-4-methylbiphenyl-3-yl)tetrahydro-1H-5,8-methanopyrazolo[1,2-a]pyridazine-1,3(2H)-dione (Ex. 8-16)
2-(4,4′-dichlorobiphenyl-3-yl)tetrahydro-1H-5,8-methanopyrazolo[1,2-a]pyridazine-1,3(2H)-dione (Ex. 8-17)
3-(4′-chloro-4-methylbiphenyl-3-yl)-4-hydroxy-6,6-dimethyl-5,6-dihydropyridin-2(1H)-one (Ex. 9-1)
3-(4′-chloro-4-methylbiphenyl-3-yl)-4-hydroxy-5,5-dimethyl-5,6-dihydropyridin-2(1H)-one (Ex. 9-2)
4-(4′-chloro-4-methylbiphenyl-3-yl)-5-hydroxy-2-azaspiro[5.5]undec-4-en-3-one (Ex. 9-3)
3-(4′-chloro-4-methylbiphenyl-3-yl)-4-hydroxy-1,6,6-trimethyl-5,6-dihydropyridin-2(1H)-one (Ex. 9-4) 9
3-(4′-chloro-4-methylbiphenyl-3-yl)-4-hydroxyquinolin-2(1H)-one (Ex. 9-5)
7-chloro-4-hydroxy-3-[4-methyl-4′-(trifluoromethyl)biphenyl-3-yl]quinolin-2(1H)-one (Ex. 9-6)
7-chloro-3-(3′,4′-dichloro-4-methylbiphenyl-3-yl)-4-hydroxyquinolin-2(1H)-one (Ex. 9-7)
3-(4′-chloro-4-methylbiphenyl-3-yl)-7-fluoro-4-hydroxyquinolin-2(1H)-one (Ex. 9-8)
7-fluoro-3-(4′-fluoro-4-methylbiphenyl-3-yl)-4-hydroxyquinolin-2(1H)-one (Ex. 9-9)
7-fluoro-4-hydroxy-3-[4-methyl-4′-(trifluoromethyl)biphenyl-3-yl]quinolin-2(1H)-one (Ex. 9-10)
7-chloro-3-(4′-chloro-4-methylbiphenyl-3-yl)-4-hydroxyquinolin-2(1H)-one (Ex. 9-11)
7-chloro-3-(4′-chloro-4,6-dimethylbiphenyl-3-yl)-4-hydroxyquinolin-2(1H)-one (Ex. 9-12)
7-chloro-3-(4,4′-dichlorobiphenyl-3-yl)-4-hydroxyquinolin-2(1H)-one (Ex. 9-13)
3-(4′-chloro-4-methylbiphenyl-3-yl)-4-hydroxy-5,5,6,6-tetramethyl-5,6-dihydropyridin-2(1H)-one (Ex. 9-14)
3-(4,4′-dichlorobiphenyl-3-yl)-4-hydroxy-5,5,6,6-tetramethyl-5,6-dihydro-2H-pyran-2-one (Ex. 10-1)
3-(4′-chloro-4-methylbiphenyl-3-yl)-4-hydroxy-5,5,6,6-tetramethyl-5,6-dihydro-2H-pyran-2-one (Ex. 10-2)
4-(4′-chloro-4-methylbiphenyl-3-yl)-2,6,6-trimethyl-1,2-oxazinane-3,5-dione (Ex. 11-1)

Saturated or unsaturated hydrocarbon radicals such as alkyl, alkanediyl or alkenyl may in each case be straight-chain or branched as far as this is possible, also in combination with heteroatoms, such as, for example, in alkoxy.

Unless indicated otherwise, optionally substituted radicals may be mono- or polysubstituted, where in the case of polysubstitution the substituents may be identical or different.

The present invention also comprises all compounds resulting from all possible combinations of the abovementioned possible, preferred and particularly preferred meanings of the substituents.

Particular embodiments of the invention additionally consist of compounds resulting from combinations of the substituent meanings disclosed directly in the examples.

The present invention likewise embraces the use of the physiologically acceptable salts of the compounds.

Physiologically acceptable salts of the compounds according to the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

The present invention furthermore provides medicaments comprising at least one compound according to the invention and at least one or more active compounds, in particular for the treatment and/or prophylaxis of diabetes, obesity, hyperlipidaemia, fatty livers and of disorders of the cardiovascular system.

The compounds according to the invention can act systemically and/or locally. For this purpose, they can be administered in a suitable manner, such as, for example, orally, parenterally, pulmonarily, nasally, sublingually, lingually, buccally, rectally, dermally, transdermally, conjunctivally, otically, as or as an implant or stent.

For these administration routes, the compounds according to the invention can be administered in suitable administration forms.

Suitable for oral administration are administration forms working according to the prior art, which release the compounds according to the invention rapidly and/or in modified form and comprise the compounds according to the invention in crystalline and/or amorphized and/or dissolved form, such as, for example, tablets (non-coated or coated tablets, for example coated with enteric, slowly dissolving or insoluble coats which control the release of the compound according to the invention), tablets which decompose rapidly in the oral cavity or films/wafers, films/lyophylizates, capsules (for example hard gelatin capsules or soft gelatin capsules), sugar-coated tablets, granules, pellets, powders, emulsions, suspensions, aerosols or solutions.

Parenteral administration can take place with circumvention of an absorption step (for example intravenous, intraarterial, intracardiac, intraspinal or intralumbar) or with involvement of an absorption (for example intramuscular, subcutaneous, intracutaneous, percutaneous or intraperitoneal). For parenteral administration, suitable administration forms are, inter alia, injection and infusion preparations in the form of solutions, suspensions, emulsions, lyophilizates or sterile powders.

Suitable for the other administration routes are, for example, pharmaceutical forms for inhalation (inter alia powder inhalers, nebulizers), nasal drops, nasal solutions, nasal sprays; tablets, films/wafers or capsules to be applied lingually, sublingually or buccally, suppositories, ear or eye preparations, vaginal capsules, aqueous suspensions (lotions, shake lotions), lipophilic suspensions, ointments, creams, transdermal therapeutic systems (such as, for example, patches), milk, pastes, foams, dusting powders, implants or stents.

The compounds according to the invention can be converted into the administration forms mentioned. This may take place in a manner known per se by mixing with inert non-toxic, pharmaceutically acceptable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and taste and/or odour corrigents.

The present invention furthermore provides medicaments comprising at least one compound according to the invention, usually together with one or more inert non-toxic, pharmaceutically suitable auxiliaries, and their use for the purposes mentioned above.

Formulation of the compounds according to the invention to give pharmaceutical products takes place in a manner known per se by converting the active ingredient(s) with the excipients customary in pharmaceutical technology into the desired administration form.

Excipients which can be employed in this connection are, for example, carrier substances, fillers, disintegrants, binders, humectants, lubricants, absorbents and adsorbents, diluents, solvents, cosolvents, emulsifiers, solubilizers, masking flavours, colorants, preservatives, stabilizers, wetting agents, salts to alter the osmotic pressure or buffers.

Reference should be made in this connection to Remington's Pharmaceutical Science, 15th ed. Mack Publishing Company, East Pennsylvania (1980).

The pharmaceutical formulations may be

in solid form, for example as tablets, coated tablets, pills, suppositories, capsules, transdermal systems or
in semisolid form, for example as ointments, creams, gels, suppositories, emulsions or
in liquid form, for example as solutions, tinctures, suspensions or emulsions.

Excipients in the context of the invention may be, for example, salts, saccharides (mono-, di-, tri-, oligo-, and/or polysaccharides), proteins, amino acids, peptides, fats, waxes, oils, hydrocarbons and derivatives thereof, where the excipients may be of natural origin or may be obtained by synthesis or partial synthesis.

Suitable for oral or peroral administration are in particular tablets, coated tablets, capsules, pills, powders, granules, pastilles, suspensions, emulsions or solutions. Suitable for parenteral administration are in particular suspensions, emulsions and especially solutions.

The present invention relates to the use of the compounds of the formulae (I), (I-1) and (I-2) for the prophylaxis and therapy of human disorders, in particular of diabetes, obesity, hyperlipidaemia, fatty livers and of disorders of the cardiovascular system.

These disorders are well-characterized in man, but also exist in other mammals.

The invention provides the compounds of the general formula (I) according to the invention, in particular also of the formulae (I-1) and (1-2), for use as a medicament.

The invention furthermore provides the use of the compounds of the general formula (I) according to the invention, in particular also of the formulae (I-1) and (1-2), for preparing a medicament.

The invention furthermore provides the use of the compounds according to the invention for treating disorders associated with proliferative processes.

The compounds according to the invention can be employed by themselves or, if required, in combination with one or more other pharmacologically active substances, as long as this combination does not lead to unwanted and unacceptable side effects. Accordingly, the present invention furthermore provides medicaments comprising at least one of the compounds according to the invention and one or more further active compounds, in particular for treatment and/or prevention of the abovementioned diseases.

### COMPARATIVE EXAMPLES

Table C.1 lists related structures of the prior art and indicates which patent discloses the preparation.

TABLE C.1

| Ex. | Structure/Name | disclosed in |
|---|---|---|
| C-1 | 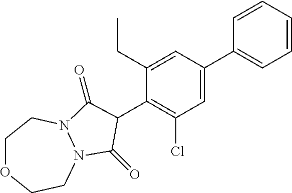 | WO 01/17973 |
| C-2 | 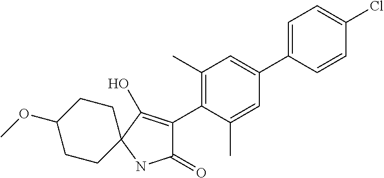 | WO 99/43649 I-1-a-2 |
| C-3 | 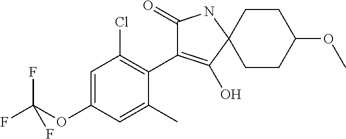 | WO 08/067873 I-a-39 |
| C-4 | 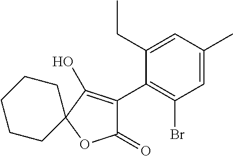 | WO 97/02243 I-a-27 |

### Examples

The compounds which form part of the subject matter of the present application and also their preparation are disclosed in the prior art.

Table 1 lists the structures of the formula I-1 of the prior art and indicates which patent discloses the preparation.

TABLE 1

| | | | Analysis ¹H-NMR: δ [ppm] |
|---|---|---|---|
| Ex. | Structure/Name | disclosed in | retention time, [M + H]⁺, Method |
| 1-1 | 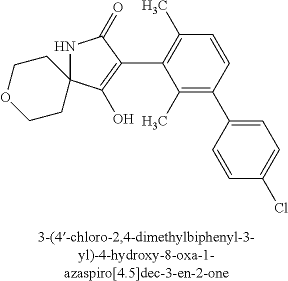 | WO 99/48869 I-1-a-15 | (300 MHz, DMSO-d₆): 1.23- 1.36 (m, 2H), 1.98 (s, 3H), 2.03- 2.19 (m, 5H), 3.62-3.76 (m, 2H), 3.80-3.91 (m, 2H), 7.05 (d, 1H), 7.13 (d, 1H), 7.26-7.35 (m, 2H), 7.45-7.53 (m, 2H), 8.40 (s, 1H), 10.89 (br. s., 1H). 1.14 min, 384, Method 1 |
| 1-2 | 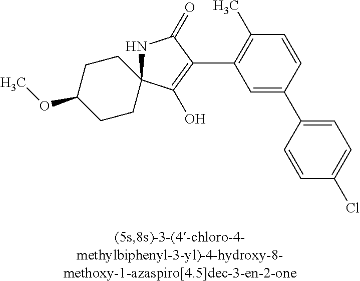 | WO 99/48869 I-1-a-16 | (300 MHz, DMSO-d₆): 1.39- 1.62 (m, 4H), 1.84-2.05 (m, 4H), 2.18 (s, 3H), 3.07-3.20 (m, 1H), 3.26 (s, 3H), 7.30 (d, 1H), 7.34 (d, 1H), 7.45-7.53 (m, 3H), 7.62-7.68 (m, 2H), 8.18 (br. s, 1H), 10.82 (br. s, 1H). 1.20 min, 398, Method 1 |
| 1-3 | 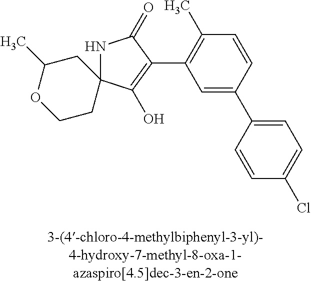 | WO 99/48869 I-1-a-17 | |
| 1-4 | 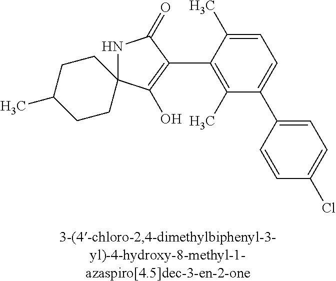 | WO 99/48869 I-1-a-2 | |
| 1-5 | 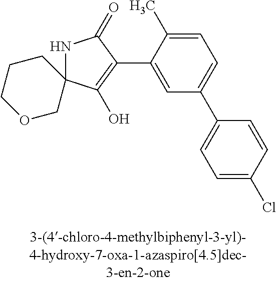 | WO 99/48869 I-1-a-21 | (300 MHz, DMSO-d₆): 1.47- 1.63 (m, 2H), 1.85-2.03 (m, 1H), 2.05-2.21 (m, 4H), 3.27- 3.40 (m, 1H), 3.44-3.52 (m, 1H), 3.67-3.74 (m, 1H), 3.80- 3.89 (m, 1H), 7.28 (d, 1H), 7.36 (d, 1H), 7.42-7.52 (m, 3H), 7.60- 7.68 (m, 2H), 7.95 (br. s., 1H), 11.08 (s, 1H). 1.14 min, 370, Method 1 |
| 1-6 | 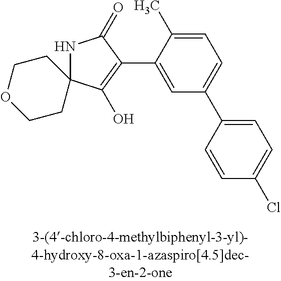 | WO 99/48869 I-1-a-22 | (300 MHz, DMSO-d₆): 1.25- 1.35 (m, 2H), 2.06-2.23 (m, 5H), 3.63-3.76 (m, 2H), 3.81- 3.91 (m, 2H), 7.31 (d, 1H), 7.36 (d, 1H), 7.46-7.53 (m, 3H), 7.62- 7.69 (m, 2H), 8.46 (s, 1H), 10.98 (br. s., 1H). 1.11 min, 370, Method 1 |
| 1-7 | 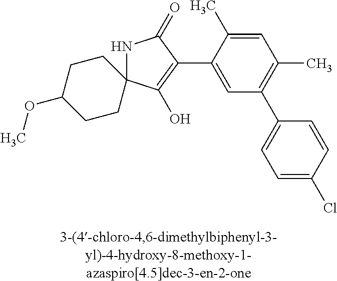 | WO 99/48869 I-1-a-25 | (300 MHz, DMSO-d₆): 1.36- 1.59 (m, 4H), 1.82-2.02 (m, 4H), 2.15 (s, 3H), 2.21 (s, 3H), 3.05-3.18 (m, 1H), 3.25 (s, 3H), 6 89 (s, 1H), 7.14 (s, 1H), 7.31- 7.38 (m, 2H), 7.44-7.52 (m, 2H), 8.14 (s, 1H), 10.73 (br. s., 1H). 1.26 min, 412, Method 1 |
| 1-8 | 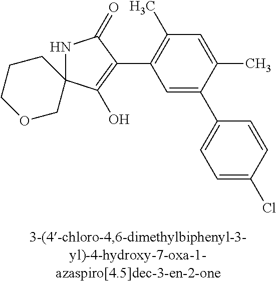 | WO 99/48869 I-1-a-27 | (400 MHz, DMSO-d₆): 1.47- 1.62 (m, 2H), 1.88-2.01 (m, 1H), 2.07-2.13 (m, 1H), 2.14 (s, 3H), 2.21 (s, 3H), 3.29-3.38 (m, 1H), 3.47 (dd, 1H), 3.71 (d, 1H), 3.81-3.88 (m, 1H), 6.90 (s, 1H), 7.15 (s, 1H), 7.33-7.37 (m, 2H), 7.46-7.51 (m, 2H), 8.14 (s, 1H), 10.86 (br. s., 1H). 1.20 min, 384, Method 1 |
| 1-9 | 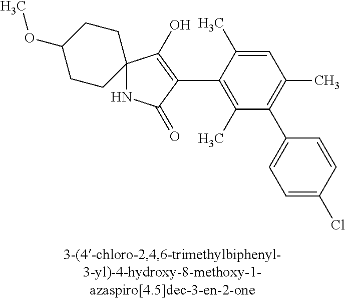 | WO 99/48869 I-1-a-3 | |
| 1-10 | 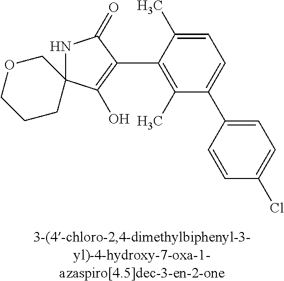 | WO 99/48869 I-1-a-7 | (400 MHz, DMSO-d₆): 1.49- 1.65 (m, 2H), 1.91-2.03 (m, 4H), 2.07-2.19 (m, 4H), 3.29- 3.40 (m, 1H), 3.45-3.53 (m, 1H), 3.68-3.75 (m, 1H), 3.82- 3.90 (m, 1H), 7.05 (d, 1H), 7.13 (d, 1H), 7.27-7.33 (m, 2H), 7.46- 7.51 (m, 2H), 8.16 (br. s., 1H), 10.89 (br. s., 1H). 1.16, 1.17 min, 384, Method 2 |
| 1-11 | 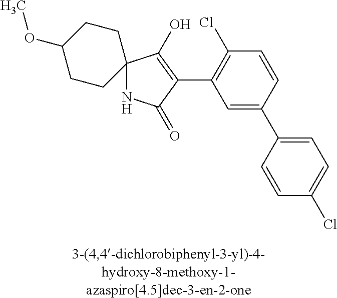 | WO 99/48869 I-1-a-9 | (300 MHz, DMSO-d₆): 1.39- 1.61 (m, 4H), 1.82-2.03 (m, 4H), 3.07-3.20 (m, 1H), 3.26 (s, 3H), 7.46-7.57 (m, 4H), 7.58- 7.64 (m, 1H), 7.65-7.74 (m, 2H), 8.22 (br. s., 1H), 11.10 (br. s., 1H). 1.21 min, 418, Method 1 |
| 1-12 | 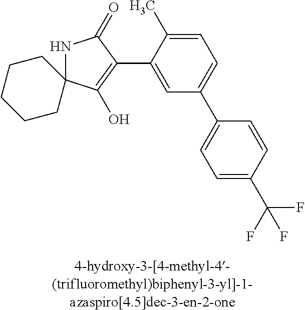 | WO 99/48869 T13 | |
| 1-13 | 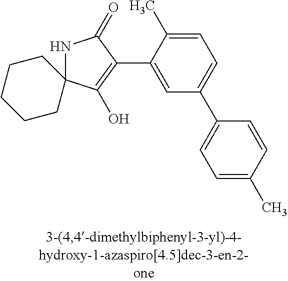 | WO 99/48869 T16 | |
| 1-14 | 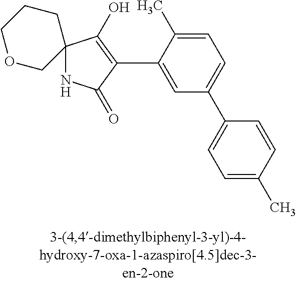 | WO 99/48869 T16 | |
| 1-15 | 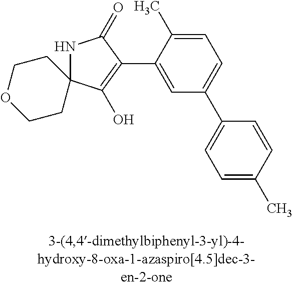 | WO 99/48869 T16 | |
| 1-16 | 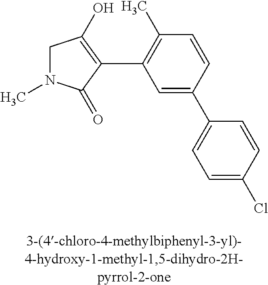 | WO 99/48869 T3 | |
| 1-17 | 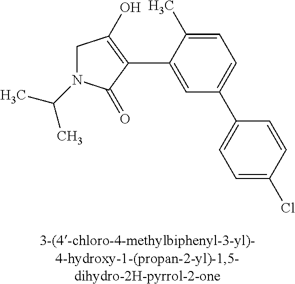 | WO 99/48869 T3 | |
| 1-18 | 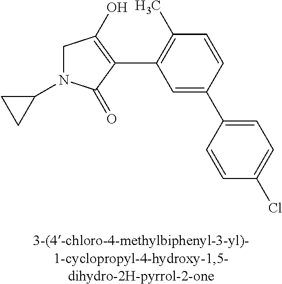 | WO 99/48869 T3 | |
| 1-19 | 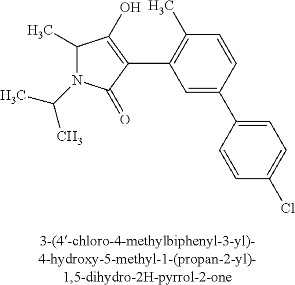 | WO 99/48869 T3 | |
| 1-20 | 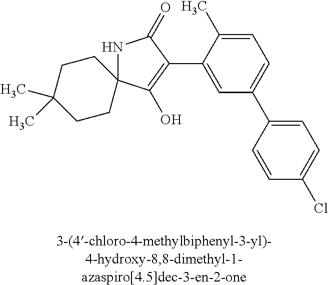 | WO 99/48869 T4 | (400 MHz, DMSO-d₆): 0.94 (s, 3H), 0.96 (s, 3H), 1.20-1.35 (m, 4H), 1.56-1.67 (m, 2H), 1.98- 2.08 (m, 2H), 2.19 (s, 3H), 7.30 (d, 1H), 7.35 (d, 1H), 7.46-7.52 (m, 3H), 7.62-7.67 (m, 2H), 8.19 (s, 1H), 10.78 (s, 1H). 1.40, 396, Method 2 |
| 1-21 | 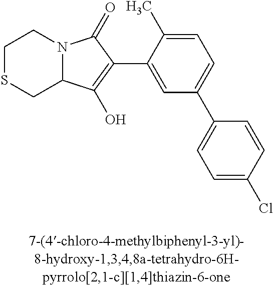 | WO 99/48869 T4 | (300 MHz, DMSO-d₆): 2.21 (s, 3H), 2.41-2.67 (m, 3H), 2.93- 3.05 (m, 1H), 3.08-3.17 (m, 1H), 4.09 (dd, 1H), 4.35-4.44 (m, 1H), 7.31 (d, 1H), 7.39 (d, 1H), 7.46-7.53 (m, 3H), 7.61- 7.68 (m, 2H), 11.25 (br. s., 1H). 1.23 min, 372, Method 1 |
| 1-22 | 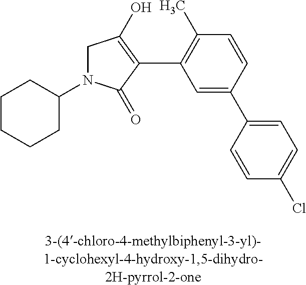 | WO 99/48869 T4 | |
| 1-23 | 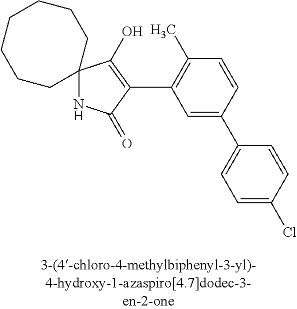 | WO 99/48869 T4 | |
| 1-24 | 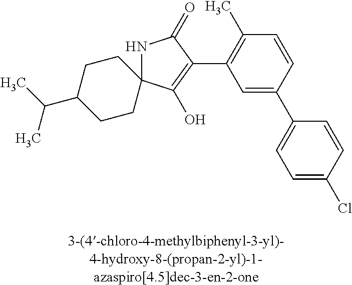 | WO 99/48869 T4 | (300 MHz, DMSO-d₆): 0.87 (s, 3H), 0.90 (s, 3H), 0.97-1.12 (m, 1H), 1.30-1.50 (m, 5H), 1.59- 1.72 (m, 2H), 1.81-1.96 (m, 2H), 2.19 (s, 3H), 7.30 (d, 1H), 7.34 (d, 1H), 7.46-7.52 (m, 3H), 7.62-7.68 (m, 2H), 8.17 (s, 1H), 10.73 (s, 1H). 1.45 min, 410, Method 1 |
| 1-25 | 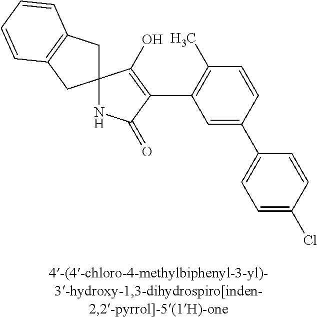 | WO 99/48869 T4 | |
| 1-26 | 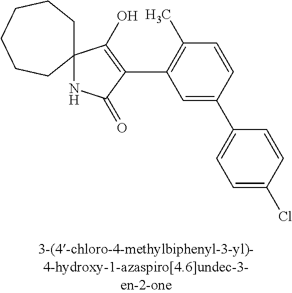 | WO 99/48869 T4 | |
| 1-27 | 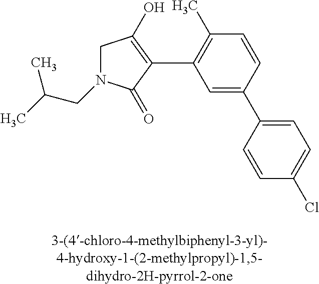 | WO 99/48869 T4 | |
| 1-28 | 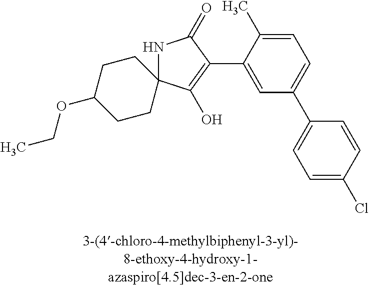 | WO 99/48869 T4 | |
| 1-29 | 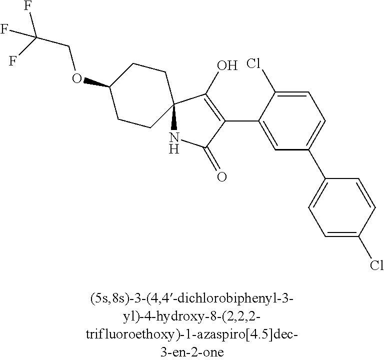 | WO 09/039975 I-1-a-18 | (400 MHz, DMSO-d₆): 1.44- 1.53 (m, 2H), 1.56-1.69 (m, 2H), 1.85-2.05 (m, 4H), 3.42- 3.52 (m, 1H), 4.10 (q, 2H), 7.49- 7.56 (m, 4H), 7.61 (dd, 1H), 7.67- 7.71 (m, 2H), 8.26 (br. s., 1H), 11.13 (s, 1H). 1.32 min, 486, Method 1 |
| 1-30 | 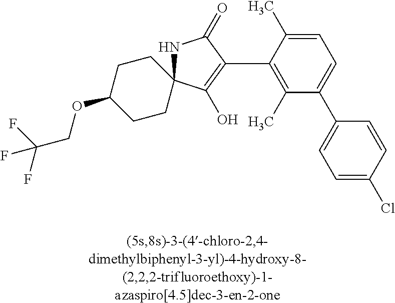 | WO 09/039975 I-1-a-25 | (300 MHz, DMSO-d₆): 1.39- 1.52 (m, 2H), 1.54-1.72 (m, 2H), 1.84-2.06 (m, 7H), 2.12 (s, 3H), 3.40-3.54 (m, 1H), 4.10 (q, 2H), 7.04 (d, 1H), 7.12 (d, 1H), 7.27-7.33 (m, 2H), 7.45-7.52 (m, 2H), 8.16 (s, 1H), 10.76 (s, 1H). 1.35 min, 480, Method 2 |
| 1-31 | 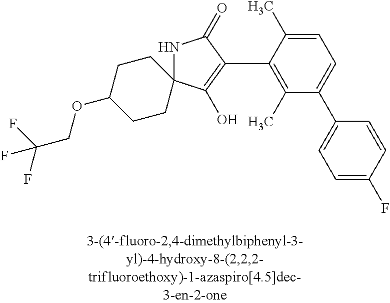 | WO 09/039975 I-1-a-3 | |
| 1-32 | 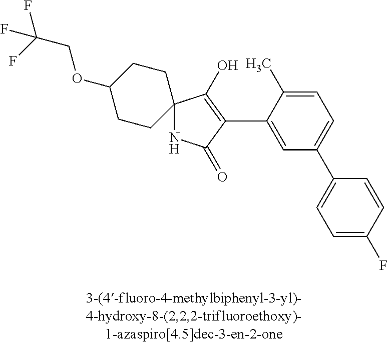 | WO 09/039975 I-1-a-31 | (300 MHz, DMSO-d₆): 1.16- 1.28 (m, 0.7H), 1.41-1.53 (m, 1.3H), 1.59-1.71 (m, 1.3H), 1.75- 2.14 (m, 4.7H), 2.18 (s, 3H), 3.40-3.53 (m, 0.7H), 3.72-3.81 (m, 0.3H), 4.00-4.17 (m, 2H), 7.22-7.33 (m, 4H), 7.43-7.49 (m, 1H), 7.61-7.69 (m, 2H), 8.20 (br. s., 1H), 10.83 (br. s., 1H). 1.32 min, 466, Method 1 |
| 1-33 | 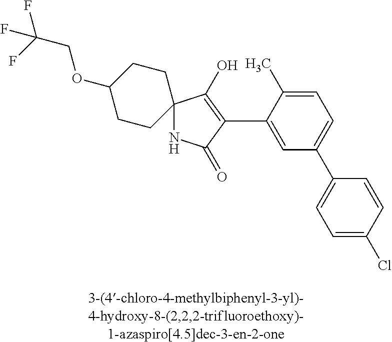 | WO 09/039975 T1 | (300 MHz, DMSO-d₆): 1.14- 1.24 (m, 0.7H), 1.40-1.53 (m, 1.3H), 1.55-1.73 (m, 1.3H), 1.79- 2.14 (m, 4.7H), 2.18 (s, 3H), 3.41-3.54 (m, 0.7H), 3.73-3.80 (m, 0.3H), 4.00-4.17 (m, 2H), 7.27-7.36 (m, 2H), 7.45-7.58 (m, 3H), 7.61-7.68 (m, 2H), 8.19 (br. s., 1H), 10.87 (br. s., 1H). 1.37 min, 480, Method 1 |
| 1-34 | 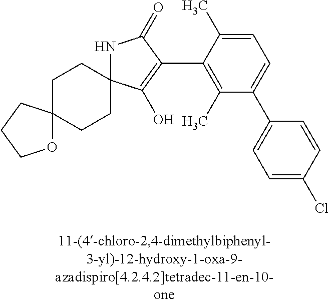 | WO 09/015801 I-1-a-1 | (300 MHz, DMSO-d₆): 1.19- 1.31 (m, 2H), 1.56-1.67 (m, 4H), 1.70-1.80 (m, 2H), 1.80- 1.92 (m, 2H), 1.98 (s, 3H), 2.09- 2.24 (m, 5H), 3.72 (t, 2H), 7.04 (d, 1H), 7.12 (d, 1H), 7.27-7.34 (m, 2H), 7.46-7.52 (m, 2H), 8.13 (s, 1H), 10.70 (s, 1H). 1.29 min, 438, Method 1 |
| 1-35 | 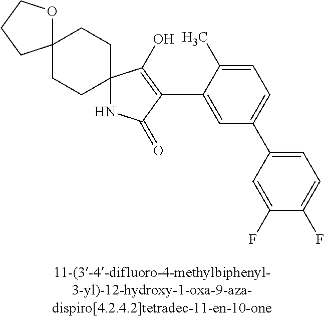 | WO 09/015801 I-1-a-45 | (300 MHz, DMSO-d₆): 1.19- 1.31 (m, 1.6H), 1.39-1.50 (m, 0.4H), 1.55-1.68 (m, 4H), 1.70- 1.92 (m, 4H), 2.10-2.24 (m, 5H), 3.67-3.77 (m, 2H), 7.29 (d, 1H), 7.35 (d, 1H), 7.44-7.53 (m, 3H), 7.64-7.74 (m, 1H), 8.10 (br. s., 1H), 10.80 (br. s., 1H). 1.23 min, 426, Method 1 |
| 1-36 | 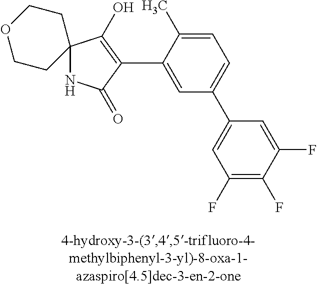 | WO 08/067911 I-1-a-28 | |
| 1-37 | 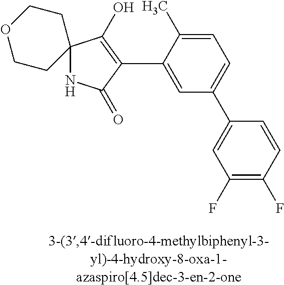 | WO 08/067911 I-1-a-3 | |
| 1-38 | 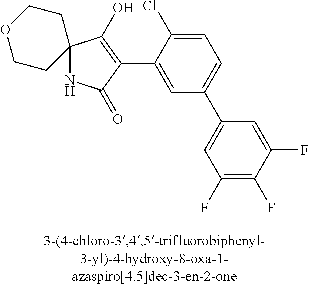 | WO 08/067911 I-1-a-30 | |
| 1-39 | 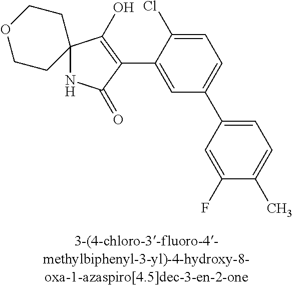 | WO 08/067911 I-1-a-45 | |
| 1-40 | 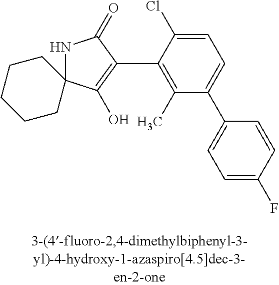 | WO 08/067911 I-1-a-53 | (300 MHz, DMSO-d₆): 1.12- 1.28 (m, 1H), 1.31-1.44 (m, 2H), 1.55-1.72 (m, 5H), 1.75- 1.92 (m, 2H), 1.97 (s, 3H), 2.12 (s, 3H), 7.03 (d, 1H), 7.10 (d, 1H), 7.20-7.34 (m, 4H), 8.06 (br. s., 1H), 10.65 (br. s., 1H). 1.25 min, 366, Method 1 |
| 1-41 | 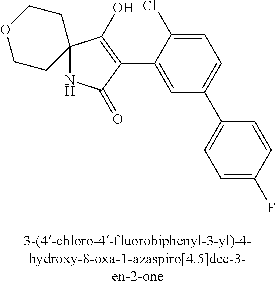 | WO 08/067911 T1 and T2 | |
| 1-42 | 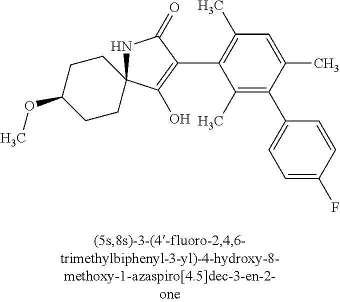 | WO 08/067910 I-1-a-5 | |
| 1-43 | 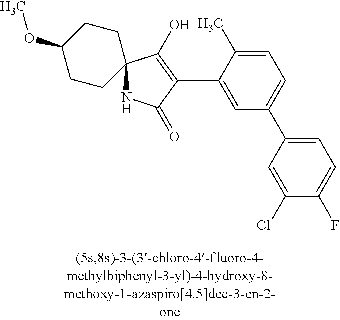 | WO 08/067910 I-a-14 | |
| 1-44 | 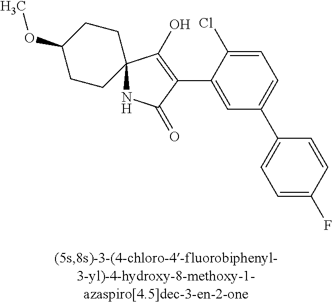 | WO 08/067910 I-a-9 | |
| 1-45 | 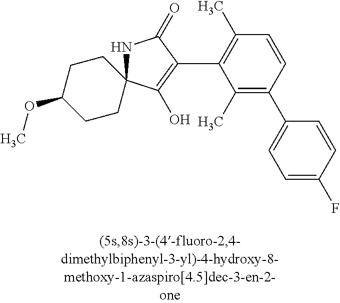 | WO 08/067910 I-a-2 | |
| 1-46 | 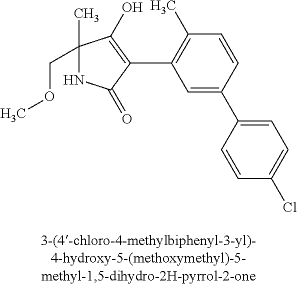 | WO 07/140881 I-a-1 | |
| 1-47 | 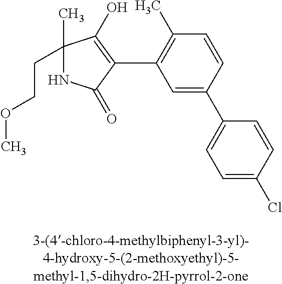 | WO 07/140881 I-a-21 | |
| 1-48 | 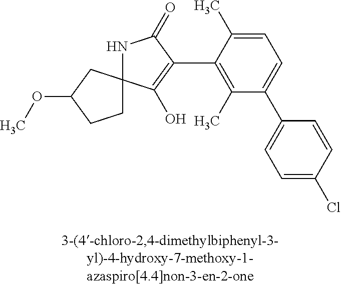 | WO 07/073856 T1 | |
| 1-49 | 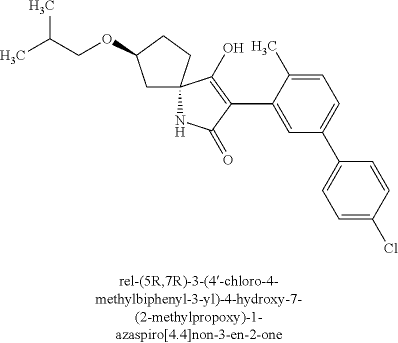 | WO 07/073856 T5 | |
| 1-50 | 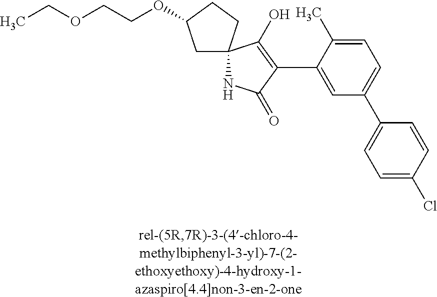 | WO 07/073856 T9 | |
| 1-51 | 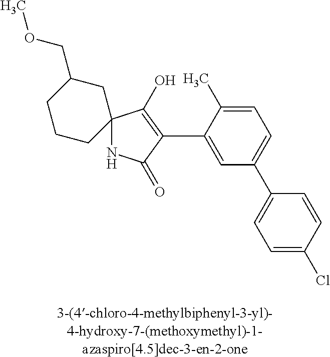 | WO 07/048545 I-1-a-20 | |
| 1-52 | 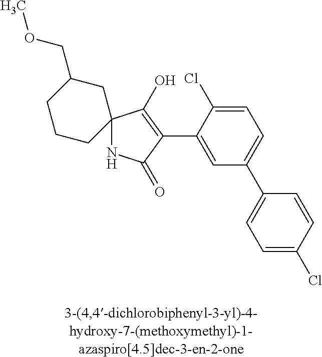 | WO 07/048545 I-1-a-24 | |
| 1-53 | 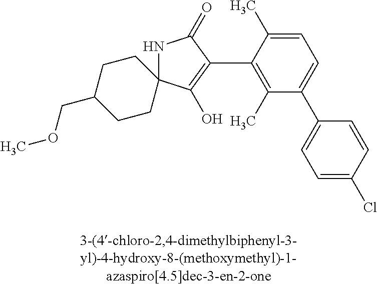 | WO 07/048545 I-1-a-28 | (300 MHz, DMSO-d₆): 1.21- 1.46 (m, 3.7H), 1.48-1.77 (m, 1.3H), 1.64-1.77 (m, 2H), 1.78- 1.96 (m, 2H), 1.98 (s, 3H), 2.13 (s, 3H), 3.16 (d, 1.4H), 3.24 (s, 2.1H), 3.26 (s, 0.9H), 3.36 (d, 0.6H), 7.04 (d, 1H), 7.12 (d, 1H), 7.26-7.34 (m, 2H), 7.45-7.52 (m, 2H), 7.98 (s, 0.3H), 8.08 (s, 0.7H), 10.68 (br. s., 1H). 1.27 min, 426, Method 1 |
| 1-54 | 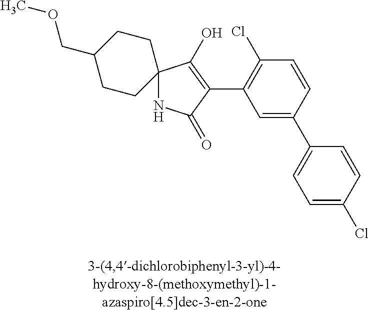 | WO 07/048545 I-1-a-34 | (400 MHz, DMSO-d₆): 1.22- 1.46 (m, 3.8H), 1.50-1.59 (m, 1.2H), 1.59-1.75 (m, 2H), 1.75- 1.93 (m, 2H), 3.16 (d, 1.6H), 3.24 (s, 2.4H), 3.26 (s, 0.6H), 3.33- 3.38 (m, 0.4H), 7.49-7.56 (m, 4H), 7.61 (dd, 1H), 7.67-7.72 (m, 2H), 8.08 (s, 0.2H), 8.18 (s, 0.8H), 11.01 (s, 0.2H), 11.05 (s, 0.8H). 1.25 min, 432, Method 1 |
| 1-55 | 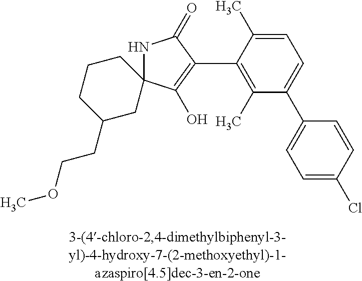 | WO 07/048545 I-1-a-42 | |
| 1-56 | 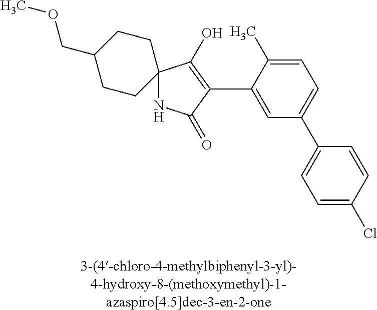 | WO 07/048545 I-1-a-5 | (300 MHz, DMSO-d₆): 1.22- 1.46 (m, 3.8H), 1.48-1.63 (m, 1.2H), 1.65-1.76 (m, 2H), 1.81- 1.96 (m, 2H), 2.19 (s, 3H), 3.16 (d, 1.6H), 3.24 (s, 2.4H), 3.27 (s, 0.6H), 3.38 (d, 0.4H), 7.30 (d, 1H), 7.34 (d, 1H), 7.45-7.53 (m, 3H), 7.61-7.68 (m, 2H), 8.05 (s, 0.2H), 8.13 (s, 0.8H), 10.77 (br. s., 1H). 1.25 min, 412, Method 1 |
| 1-57 | 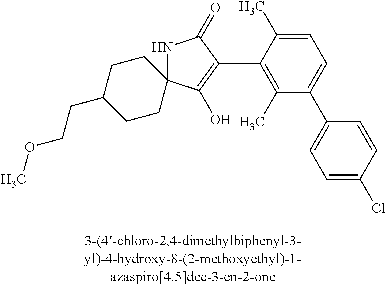 | WO 07/048545 I-1-a-53 | (400 MHz, DMSO-d₆): 1.21- 1.49 (m, 6H), 1.50-1.72 (m, 3H), 1.74-1.93 (m, 2H), 1.98 (s, 2.4H), 2.05 (s, 0.6H), 2.12 (s, 2.4H), 2.16 (s, 0.6H), 3.22 (s, 2.4H), 3.23 (s, 0.6H), 3.32-3.39 (m, 2H), 7.04 (d, 1H), 7.12 (d, 1H), 7.27-7.34 (m, 2H), 7.45- 7.52 (m, 2H), 7.97 (br. s., 0.2H), 8.09 (br. s., 0.8H), 10.64 (br. s., 0.8H), 10.75 (br. s., 0.2H). 1.31 min, 440, Method 1 |
| 1-58 | 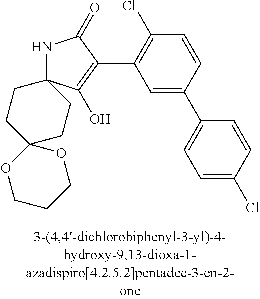 | WO 06/089633 I-1-a-15 | |
| 1-59 | 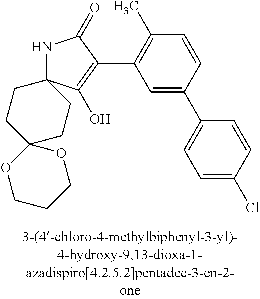 | WO 06/089633 I-1-a-16 | |
| 1-60 | 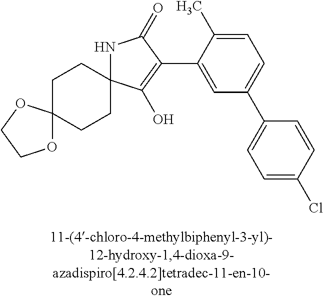 | WO 06/089633 I-1-a-27 | (300 MHz, DMSO-d₆): 1.38- 1.49 (m, 2H), 1.65-1.76 (m, 2H), 1.82-2.03 (m, 3H), 2.06- 2.22 (m, 4H), 3.89 (s, 4H), 7.27- 7.36 (m, 2H), 7.46-7.54 (m, 3H), 7.61-7.69 (m, 2H), 8.25 (br. s., 1H), 10.90 (br. s., 1H). 1.20, 426, Method 2 |
| 1-61 | 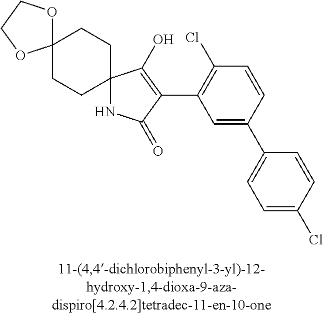 | WO 06/089633 I-1-a-49 | |
| 1-62 | 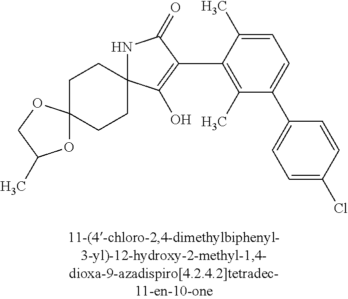 | WO 06/089633 I-1-a-51 | |
| 1-63 | 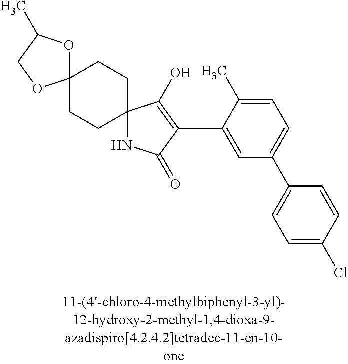 | WO 06/089633 I-1-a-52 | |
| 1-64 | 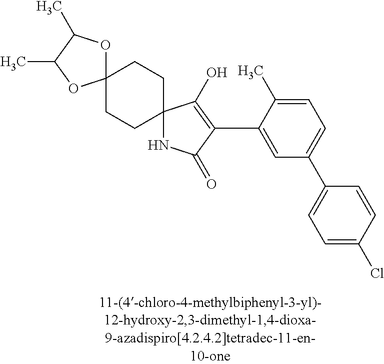 | WO 06/089633 I-1-a-54 | |
| 1-65 | 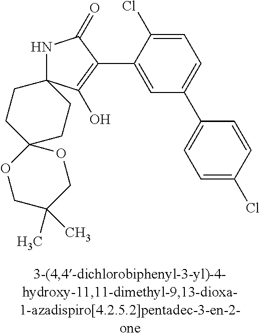 | WO 06/089633 I-1-a-64 | |
| 1-66 | 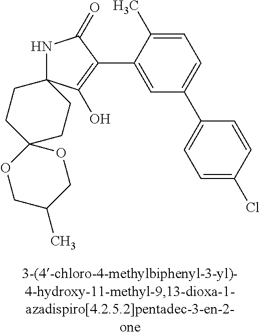 | WO 06/089633 I-1-a-65 | |
| 1-67 | 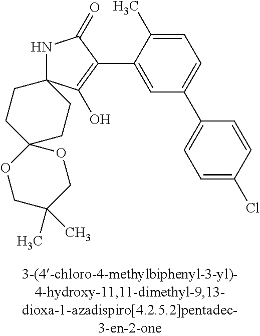 | WO 06/089633 I-1-a-67 | |
| 1-68 | 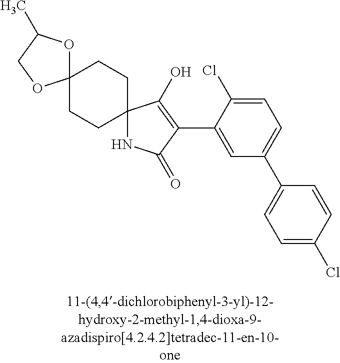 | WO 06/089633 I-1-a-76 | |
| 1-69 | 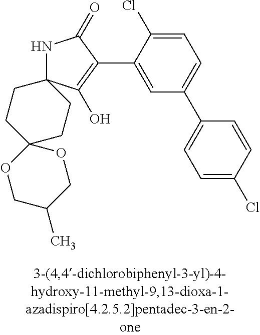 | WO 06/089633 I-1-a-79 | |
| 1-70 | 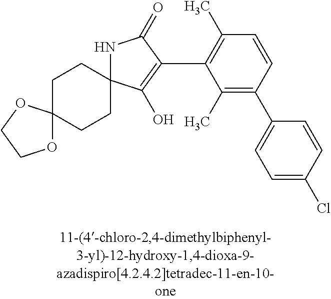 | WO 06/089633 T1 | |
| 1-71 | 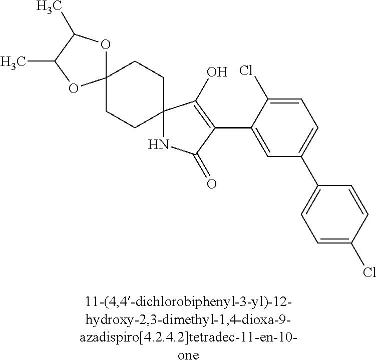 | WO 06/089633 T3 | |
| 1-72 | 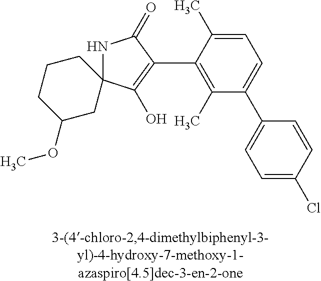 | WO 06/000355 T1 | |
| 1-73 | 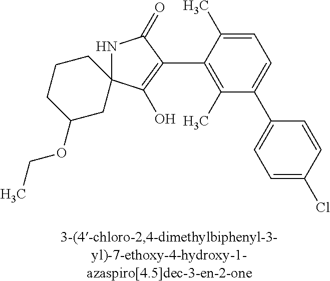 | WO 06/000355 T2 | |
| 1-74 | 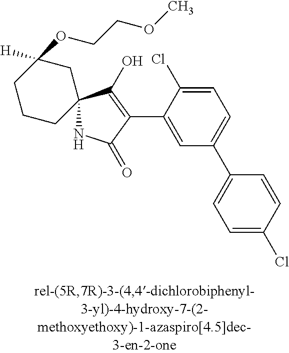 | WO 06/000355 T8 | |
| 1-75 | 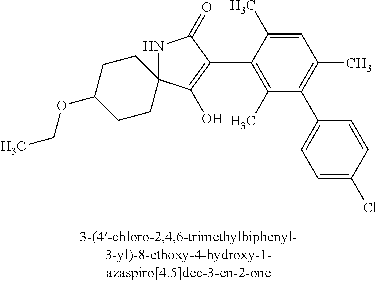 | WO 03/059065 I-1-a-16 | |
| 1-76 | 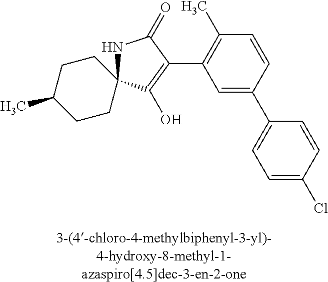 | WO 03/059065 I-1-a-17 | (300 MHz, DMSO-d₆): 0.91 (d, 3H), 1.29-1.44 (m, 5H), 1.55- 1.68 (m, 2H), 1.82-1.98 (m, 2H), 2.19 (s, 3H), 7.30 (d, 1H), 7.34 (d, 1H), 7.45-7.53 (m, 3H), 7.61-7.69 (m, 2H), 8.13 (s, 1H), 10.75 (br. s., 1H). 1.34 min, 382, Method 1 |
| 1-77 | 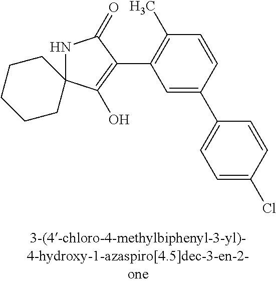 | WO 03/059065 I-1-a18 | (300 MHz, DMSO-d₆): 1.10- 1.29 (m, 1H), 1.33-1.43 (m, 2H), 1.52-1.73 (m, 5H), 1.78- 1.92 (m, 2H), 2.19 (s, 3H), 7.29 (d, 1H), 7.35 (d, 1H), 7.45-7.52 (m, 3H), 7.62-7.68 (m, 2H), 8.10 (br. s., 1H), 10.82 (br. s, 1H). 1.32 min, 368, Method 1 |
| 1-78 | 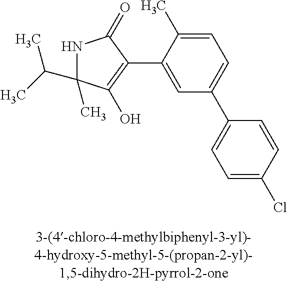 | WO 03/059065 I-1-a-19 | |
| 1-79 | 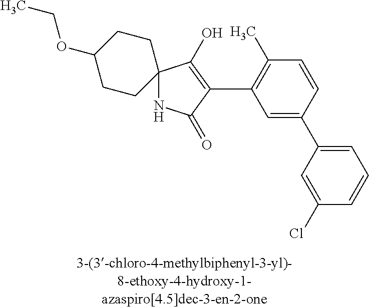 | WO 03/059065 I-1-a-22 | |
| 1-80 | 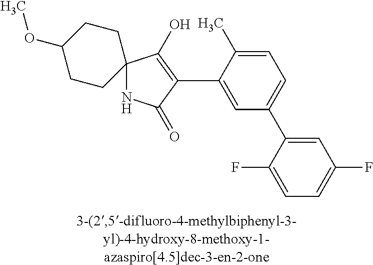 | WO 03/059065 I-1-a-29 | |
| 1-81 | 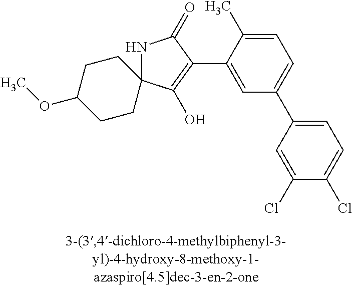 | WO 03/059065 I-1-a-31 | |
| 1-82 | 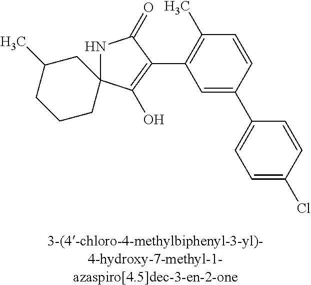 | WO 03/059065 I-1-a-33 | |
| 1-83 | 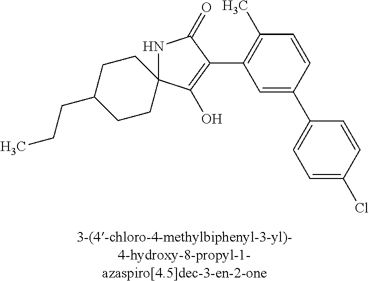 | WO 03/059065 I-1-a-35 | |
| 1-84 | 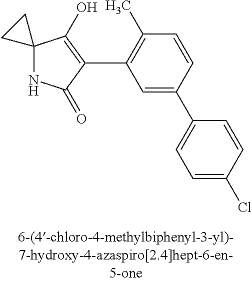 | WO 03/059065 I-1-a-36 | |
| 1-85 | 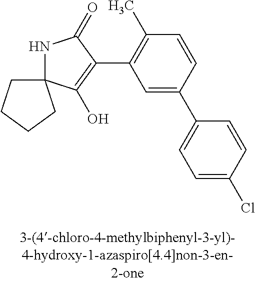 | WO 03/059065 I-1-a-37 | (300 MHz, DMSO-d₆): 1.54- 1.90 (m, 6H), 1.99-2.13 (m, 2H), 2.19 (s, 3H), 7.31 (d, 1H), 7.35 (d, 1H), 7.45-7.52 (m, 3H), 7.62-7.69 (m, 2H), 7.92 (s, 1H), 10.82 (br. s., 1H). 1.25 min, 354, Method 1 |
| 1-86 | 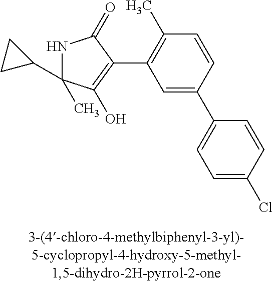 | WO 03/059065 I-1-a-39 | |
| 1-87 | 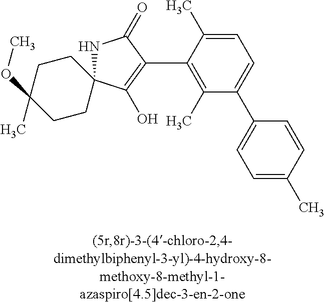 | PCT/EP/2009 008260 I-a-10 | (300 MHz, DMSO-d₆): 1.08 (s, 3H), 1.10-1.21 (m, 2H), 1.57- 1.81 (m, 4H), 1.98 (s, 3H), 2.01- 2.11 (m, 2H), 2.12 (s, 3H), 3.08 (s, 3H), 7.04 (d, 1H), 7.12 (d, 1H), 7.27-7.33 (m, 2H), 7.46- 7.52 (m, 2H), 8.14 (s, 1H), 10.72 (s, 1H). 1.27 min, 426, Method 1 |
| 1-88 | 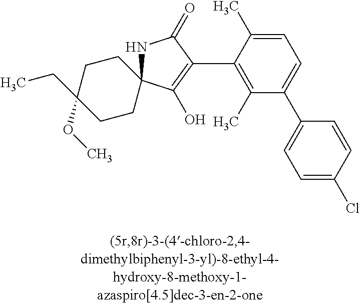 | PCT/EP/2009 008260 I-a-34 | |
| 1-89 | 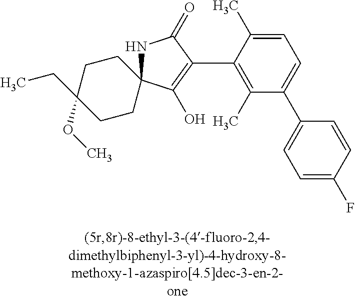 | PCT/EP/2009 008260 T3 | |

Method 1 (UPLC-MS): instrument: Waters Acquity UPLC-MS SQD 3001; column: Acquity UPLC BEH C18 1.7 50×2.1 mm; mobile phase A: water+0.1% formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nM.

Method 2 (UPLC-MS): instrument: Waters Acquity UPLC-MS ZQ4000; column: Acquity UPLC BEH C18 1.7 50×2.1 mm; mobile phase A: water+0.05% formic acid, mobile phase B: acetonitrile+0.05% formic acid; gradient: 0-1.6 min 1-99% B, 1.6-2.0 min 99% B; flow rate 0.8 ml/min; temperature: 60° C.; injection: 2 μl; DAD scan: 210-400 nM.

Table 2 lists the structures of the formula I-2 of the prior art and indicates which patent discloses the preparation.

TABLE 2

| | | | Analysis |
|---|---|---|---|
| | | | ¹H-NMR: δ [ppm] |
| Ex. | Structure/Name | disclosed in | retention time, [M + H]⁺, Method |
| 2-1 | 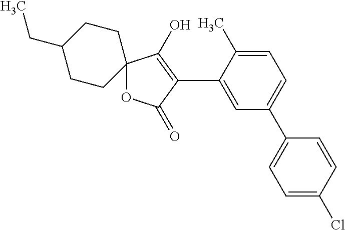 | WO 03/059065 I-2-a-12 | |
| | 3-(4′-chloro-4-methylbiphenyl-3-yl)- | | |
| | 8-ethyl-4-hydroxy-1- | | |
| | oxaspiro[4.5]dec-3-en-2-one | | |
| 2-2 | 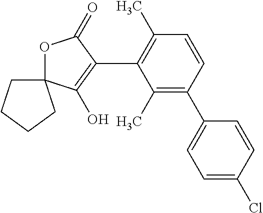 | WO 03/059065 I-2-a-19 | |
| | 3-(4′-chloro-2,4-dimethylbiphenyl-3- | | |
| | yl)-4-hydroxy-1-oxaspiro[4.4]non- | | |
| | 32--en-2-one | | |
| 2-3 | 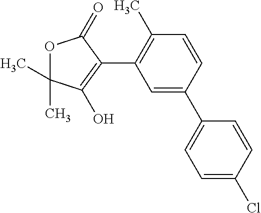 | WO 03/059065 I-2-a-2 | |
| | 3-(4′-chloro-4-methylbiphenyl-3-yl)- | | |
| | 4-hydroxy-5,5-dimethylfuran-2(5H)- | | |
| | one | | |
| 2-4 | 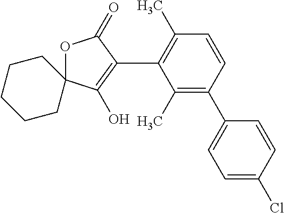 | WO 03/059065 I-2-a-20 | |
| | 3-(4′-chloro-2,4-dimethylbiphenyl-3- | | |
| | yl)-4-hydroxy-1-oxaspiro[4.5]dec-3- | | |
| | en-2-one | | |
| 2-5 | 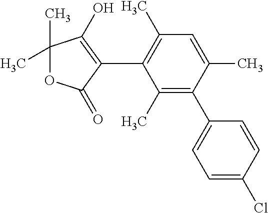 | WO 03/059065 I-2-a-21 | |
| | 3-(4′-chloro-2,4,6-trimethylbiphenyl- | | |
| | 3-yl)-4-hydroxy-5,5-dimethylfuran- | | |
| | 2(5H)-one | | |
| 2-6 | 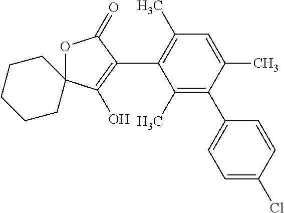 | WO 03/059065 I-2-a-22 | |
| | 3-(4′-chloro-2,4,6-trimethylbiphenyl- | | |
| | 3-yl)-4-hydroxy-1-oxaspiro[4.5]dec- | | |
| | 3-en-2-one | | |
| 2-7 | 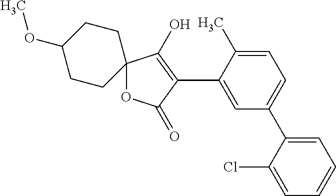 | WO 03/059065 I-2-a-32 | |
| | 3-(2′-chloro-4-methylbiphenyl-3-yl)- | | |
| | 4-hydroxy-8-methoxy-1- | | |
| | oxaspiro[4.5]dec-3-en-2-one | | |
| 2-8 | 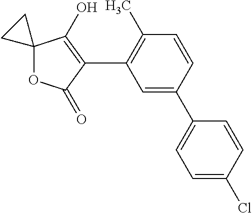 | WO 03/059065 I-2-a-6 | |
| | 6-(4′-chloro-4-methylbiphenyl-3-yl)- | | |
| | 7-hydroxy-4-oxaspiro[2.4]hept-6-en- | | |
| | 5-one | | |
| 2-9 | 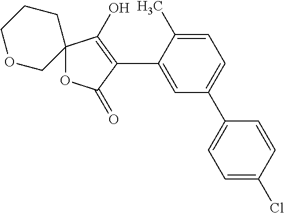 | WO 03/059065 I-2-a-7 | |
| | 3-(4′-chloro-4-methylbiphenyl-3-yl)- | | |
| | 4-hydroxy-1,7-dioxaspiro[4.5]dec-3- | | |
| | en-2-one | | |
| 2-10 | 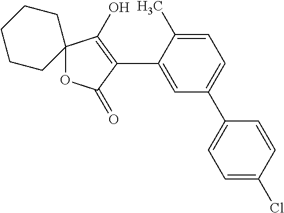 | WO 03/059065 I-2-a-8 | |
| | 3-(4′-chloro-4-methylbiphenyl-3-yl)- | | |
| | 4-hydroxy-1-oxaspiro [4.5]dec-3-en- | | |
| | 2-one | | |
| 2-11 | 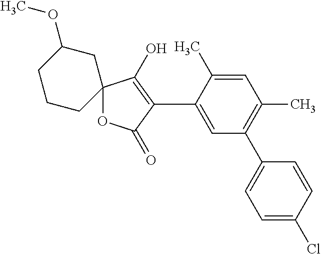 | WO 06/000355 I-2-a-2 | |
| | 3-(4′-chloro-4,6-dimethylbiphenyl-3- | | |
| | yl)-4-hydroxy-7-methoxy-1- | | |
| | oxaspiro[4.5]dec-3-en-2-one | | |
| 2-12 | 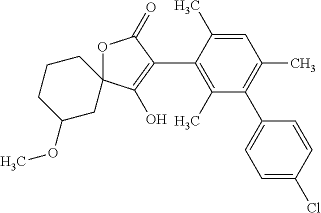 | WO 06/000355 I-2-a-30 | |
| | 3-(4′-chloro-2,4,6-trimethylbiphenyl- | | |
| | 3-yl)-4-hydroxy-7-methoxy-1- | | |
| | oxaspiro[4.5]dec-3-en-2-one | | |
| 2-13 | 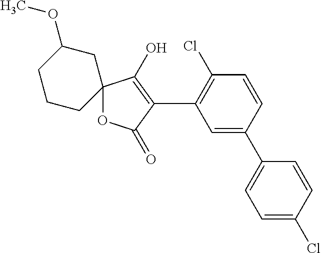 | WO 06/000355 I-2-a-31 | |
| | 3-(4,4′-dichlorobiphenyl-3-yl)-4- | | |
| | hydroxy-7-methoxy-1- | | |
| | oxaspiro[4.5]dec-3-en-2-one | | |
| 2-14 | 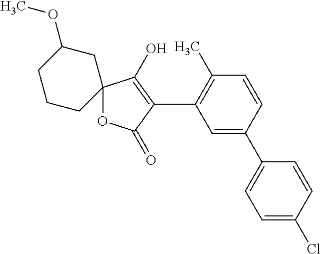 | WO 06/000355 I-2-a-32 | |
| | 3-(4′-chloro-4-methylbiphenyl-3-yl)- | | |
| | 4-hydroxy-7-methoxy-1- | | |
| | oxaspiro[4.5]dec-3-en-2-one | | |
| 2-15 | 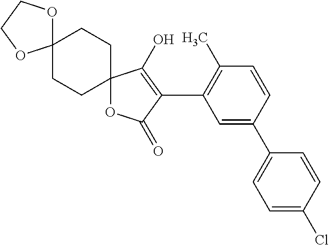 | WO 06/089633 I-2-a-8 | |
| | 11-(4′-chloro-4-methylbiphenyl-3-yl)- | | |
| | 12-hydroxy-1,4,9-trioxadi- | | |
| | spiro[4.2.4.2]tetradec-11-en-10-one | | |
| 2-16 | 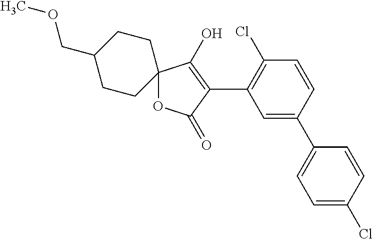 | WO 07/048545 I-2-a-16 | |
| | 3-(4,4′-dichlorobiphenyl-3-yl)-4- | | |
| | hydroxy-8-(methoxymethyl)-1- | | |
| | oxaspiro[4.5]dec-3-en-2-one | | |
| 2-17 | 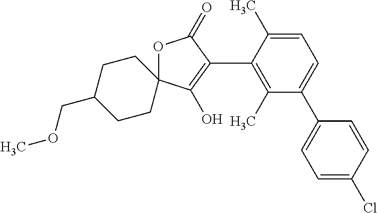 | WO 07/048545 I-2-a-17 | |
| | 3-(4′-chloro-2,4-dimethylbiphenyl-3- | | |
| | yl)-4-hydroxy-8-(methoxymethyl)-1- | | |
| | oxaspiro[4.5]dec-3-en-2-one | | |
| 2-18 | 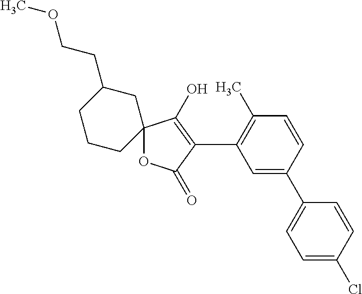 | WO 07/048545 I-2-a-18 | |
| | 3-(4′-chloro-4-methylbiphenyl-3-yl)- | | |
| | 4-hydroxy-7-(2-methoxyethyl)-1- | | |
| | oxaspiro[4.5]dec-3-en-2-one | | |
| 2-19 | 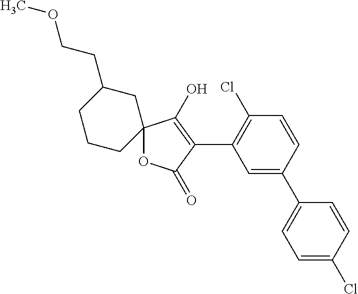 | WO 07/048545 I-2-a-26 | |
| | 3-(4,4′-dichlorobiphenyl-3-yl)-4- | | |
| | hydroxy-7-(2-methoxyethyl)-1- | | |
| | oxaspiro[4.5]dec-3-en-2-one | | |
| 2-20 | 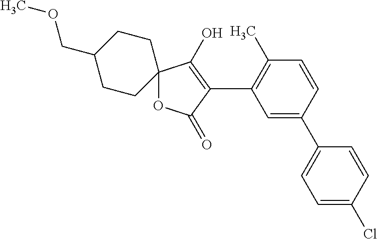 | WO 07/048545 I-2-a-8 | |
| | 3-(4′-chloro-4-methylbiphenyl-3-yl)- | | |
| | 4-hydroxy-8-(methoxymethyl)-1- | | |
| | oxaspiro[4.5]dec-3-en-2-one | | |
| 2-21 | 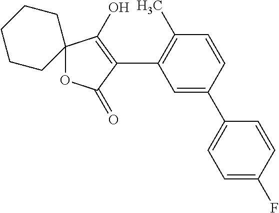 | WO 08/067911 I-2-a-15 | |
| | 3-(4′-fluoro-4-methylbiphenyl-3-yl)- | | |
| | 4-hydroxy-1-oxaspiro[4.5]dec-3-en- | | |
| | 2-one | | |
| 2-22 | 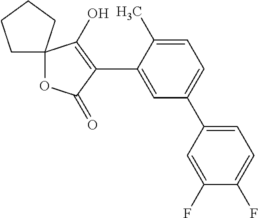 | WO 08/067911 I-2-a-3 | |
| | 3-(3′,4′-difluoro-4-methylbiphenyl-3- | | |
| | yl)-4-hydroxy-1-oxaspiro[4.4]non-3- | | |
| | en-2-one | | |
| 2-23 | 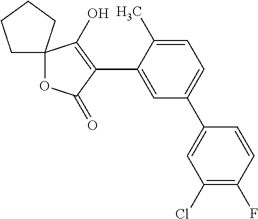 | WO 08/067911 I-2-a-4 | |
| | 3-(3′-chloro-4′-fluoro-4- | | |
| | methylbiphenyl-3-yl)-4-hydroxy-1- | | |
| | oxaspiro[4.4]non-3-en-2-one | | |
| 2-24 | 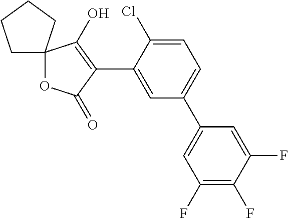 | WO 08/067911 I-2-a-6 | |
| | 3-(4-chloro-3′,4′,5′-trifluorobiphenyl- | | |
| | 3-yl)-4-hydroxy-1-oxaspiro[4.4]non- | | |
| | 3-en-2-one | | |
| 2-25 | 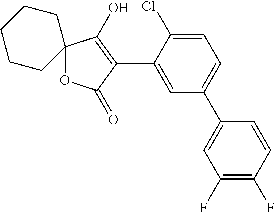 | WO 08/067911 I-2-a-8 | |
| | 3-(4-chloro-3′,4′-difluorobiphenyl-3- | | |
| | yl)-4-hydroxy-1-oxaspiro[4.5]dec-3- | | |
| | en-2-one | | |
| 2-26 | 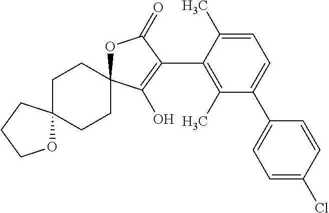 | WO 09/015801 I-2-a-19 | |
| | (5s,8r)-3-(4′-chloro-2,4- | | |
| | dimethylbiphenyl-3-yl)-4-hydroxy- | | |
| | 1,9-dioxadispiro[4.2.4.2]tetradec-3- | | |
| | en-2-one | | |
| 2-27 | 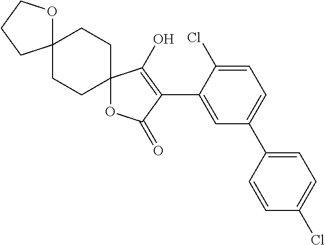 | WO 09/015801 I-2-a-21 | |
| | 3-(4,4′-dichlorobiphenyl-3-yl)-4- | | |
| | hydroxy-1,9-dioxadi- | | |
| | spiro[4.2.4.2]tetradec-3-en-2-one | | |
| 2-28 | 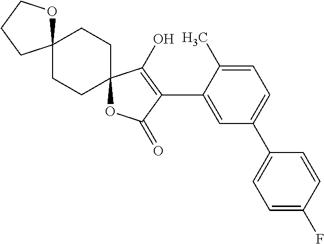 | WO 09/015801 I-2-a-22 | |
| | (5r,8s)-3-(4′-fluoro-4- | | |
| | methylbiphenyl-3-yl)-4-hydroxy-1,9- | | |
| | dioxadi-spiro[4.2.4.2]tetradec-3-en-2- | | |
| | one | | |
| 2-29 | 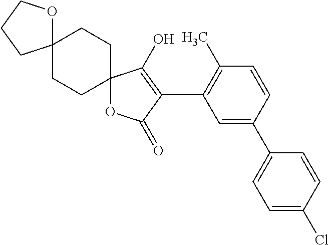 | WO 09/015801 I-2-a-25 | |
| | 3-(4′-chloro-4-methylbiphenyl-3-yl)- | | |
| | 4-hydroxy-1,9-dioxadi- | | |
| | spiro[4.2.4.2]tetradec-3-en-2-one | | |
| 2-30 | 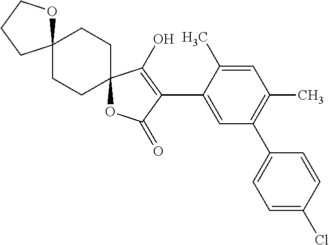 | WO 09/015801 I-2-a-27 | (300 MHz, DMSO-d₆): 1.58- 1.81 (m, 8H), 1.83-2.07 (m, 4H), 2.16 (s, 3H), 2.22 (s, 3H), 3.73 (t, 2H), 6.95 (s, 1H), 7.18 (s, 1H), 7.32-7.38 (m, 2H), 7.45- 7.52 (m, 2H), 12.19 (br. s., 1H). 1.36 min, 439, Method 1 |
| | (5r,8s)-3-(4′-chloro-4,6-dimethyl- | | |
| | biphenyl-3-yl)-4-hydroxy-1,9-dioxa- | | |
| | dispiro[4.2.4.2]tetradec-3-en-2-one | | |
| 2-31 | 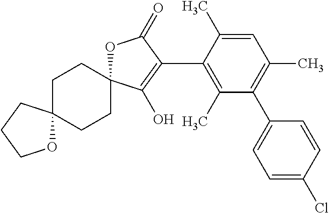 | WO 09/015801 I-2-a-42 | |
| | (5r,8s)-3-(4′-chloro-2,4,6- | | |
| | trimethylbiphenyl-3-yl)-4-hydroxy- | | |
| | 1,9-dioxadispiro[4.2.4.2]tetradec-3- | | |
| | en-2-one | | |
| 2-32 | 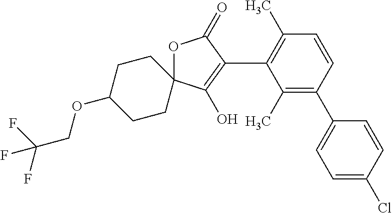 | WO 09/039975 I-2-a-6 | 1.43, 1.46 min, 481, Method 1 |
| | 3-(4′-chloro-2,4-dimethylbiphenyl-3- | | |
| | yl)-4-hydroxy-8-(2,2,2- | | |
| | trifluoroethoxy)-1-oxaspiro[4.5]dec- | | |
| | 3-en-2-one | | |
| 2-33 | 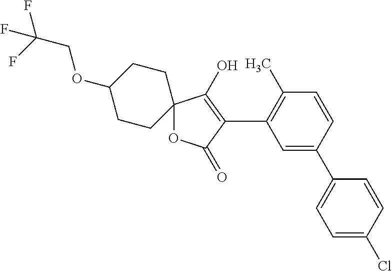 | WO 09/039975 I-2-a-7 | (300 MHz, DMSO-d₆): 1.43- 1.62 (m, 2H), 1.63-1.85 (m, 2H), 1.92-2.03 (m, 2H), 2.04- 2.24 (m, 5H), 3.82-3.90 (m, 0.5H) 3.98-4.19 (m, 2.5H), 7.32- 7.37 (m, 1H), 7.42 (d, 1H), 7.47- 7.58 (m, 3H), 7.64-7.70 (m, 2H), 12.35 (br. s., 1H). 1.40, 1.42 min, 467 Method 1 |
| | 3-(4′-chloro-4-methylbiphenyl-3-yl)- | | |
| | 4-hydroxy-8-(2,2,2-trifluoroethoxy)- | | |
| | 1-oxaspiro[4.5]dec-3-en-2-one | | |
| 2-34 | 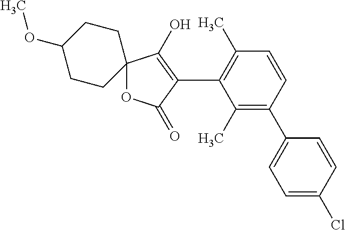 | WO 99/48869 I-2-a-2 | |
| | 3-(4′-chloro-2,4-dimethylbiphenyl-3- | | |
| | yl)-4-hydroxy-8-methoxy-1- | | |
| | oxaspiro[4.5]dec-3-en-2-one | | |
| 2-35 | 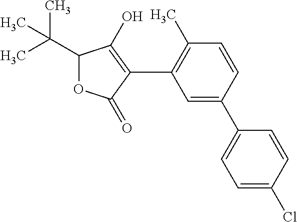 | WO 99/48869 T 25 | |
| | 5-tert-butyl-3-(4′-chloro-4- | | |
| | methylbiphenyl-3-yl)-4- | | |
| | hydroxyfuran-2(5H)-one | | |
| 2-36 | 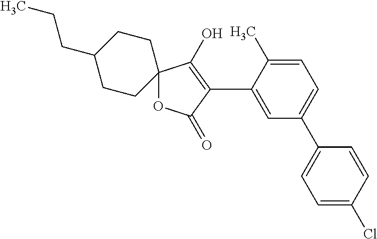 | WO 99/48869 T 25 | |
| | 3-(4′-chloro-4-methylbiphenyl-3-yl)- | | |
| | 4-hydroxy-8-propyl-1- | | |
| | oxaspiro[4.5]dec-3-en-2-one | | |
| 2-37 | 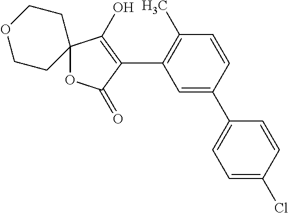 | WO 99/48869 T 25 | |
| | 3-(4′-chloro-4-methylbiphenyl-3-yl)- | | |
| | 4-hydroxy-1,8-dioxaspiro[4.5]dec-3- | | |
| | en-2-one | | |
| 2-38 | 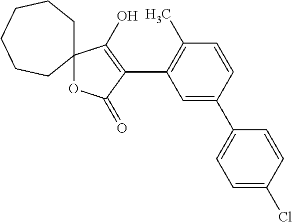 | WO 99/48869 T 25 | |
| | 3-(4′-chloro-4-methylbiphenyl-3-yl)- | | |
| | 4-hydroxy-1-oxaspiro[4.6]undec-3- | | |
| | en-2-one | | |
| 2-39 | 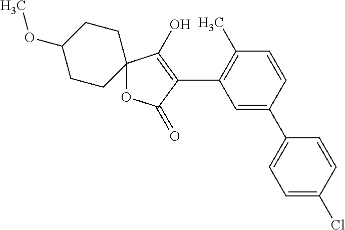 | WO 99/48869 T 25 | |
| | 3-(4′-chloro-4-methylbiphenyl-3-yl)- | | |
| | 4-hydroxy-8-methoxy-1- | | |
| | oxaspiro[4.5]dec-3-en-2-one | | |
| 2-40 | 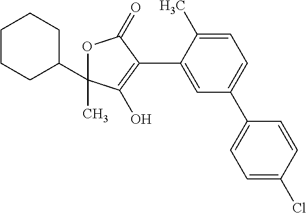 | WO 99/48869 T 25 | |
| | 3-(4′-chloro-4-methylbiphenyl-3-yl)- | | |
| | 5-cyclohexyl-4-hydroxy-5- | | |
| | methylfuran-2(5H)-one | | |
| 2-41 | 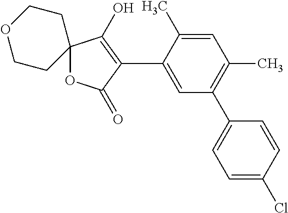 | WO 99/48869 T 26 | |
| | 3-(4′-chloro-4,6-dimethylbiphenyl-3- | | |
| | yl)-4-hydroxy-1,8- | | |
| | dioxaspiro[4.5]dec-3-en-2-one | | |
| 2-42 | 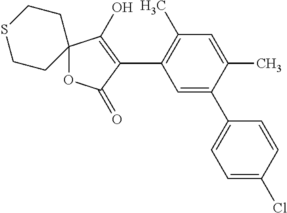 | WO 99/48869 T 26 | |
| | 3-(4′-chloro-4,6-dimethylbiphenyl-3- | | |
| | yl)-4-hydroxy-1-oxa-8- | | |
| | thiaspiro[4.5]dec-3-en-2-one | | |
| 2-43 | 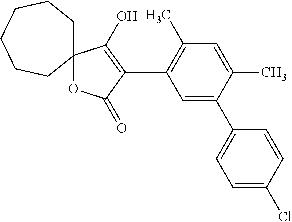 | WO 99/48869 T 26 | |
| | 3-(4′-chloro-4,6-dimethylbiphenyl-3- | | |
| | yl)-4-hydroxy-1-oxaspiro[4.6]undec- | | |
| | 3-en-2-one | | |
| 2-44 | 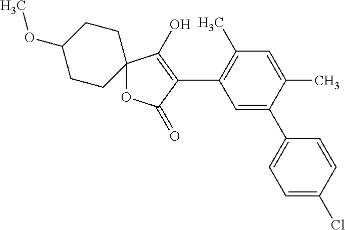 | WO 99/48869 T 26 | |
| | 3-(4′-chloro-4,6-dimethylbiphenyl-3- | | |
| | yl)-4-hydroxy-8-methoxy-1- | | |
| | oxaspiro[4.5]dec-3-en-2-one | | |
| 2-45 | 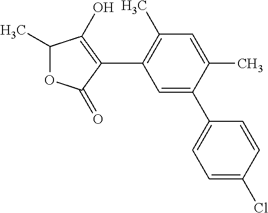 | WO 99/48869 T 26 | |
| | 3-(4′-chloro-4,6-dimethylbiphenyl-3- | | |
| | yl)-4-hydroxy-5-methylfuran-2(5H)- | | |
| | one | | |
| 2-46 | 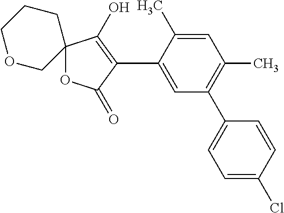 | WO 99/48869 T 26 | |
| | 3-(4′-chloro-4,6-dimethylbiphenyl-3- | | |
| | yl)-4-hydroxy-1,7- | | |
| | dioxaspiro[4.5]dec-3-en-2-one | | |
| 2-47 | 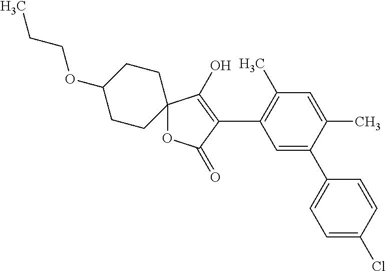 | WO 99/48869 T 26 | |
| | 3-(4′-chloro-4,6-dimethylbiphenyl-3- | | |
| | yl)-4-hydroxy-8-propoxy-1- | | |
| | oxaspiro[4.5]dec-3-en-2-one | | |
| 2-48 | 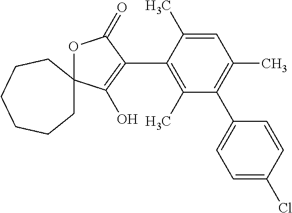 | WO 99/48869 T 27 | |
| | 3-(4′-chloro-2,4,6-trimethylbiphenyl- | | |
| | 3-yl)-4-hydroxy-1- | | |
| | oxaspiro[4.6]undec-3-en-2-one | | |
| 2-49 | 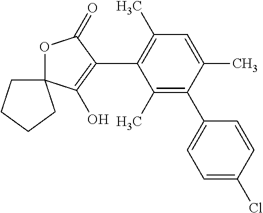 | WO 99/48869 T 27 | |
| | 3-(4′-chloro-2,4,6-trimethylbiphenyl- | | |
| | 3-yl)-4-hydroxy-1-oxaspiro[4.4]non- | | |
| | 3-en-2-one | | |
| 2-50 | 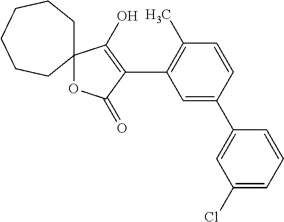 | WO 99/48869 T 28 | |
| | 3-(3′-chloro-4-methylbiphenyl-3-yl)- | | |
| | 4-hydroxy-1-oxaspiro[4.6]undec-3- | | |
| | en-2-one | | |

### Method 1 (UPLC-MS):

instrument: Waters Acquity UPLC-MS SQD 3001; column: Acquity UPLC BEH C18 1.7 50×2.1 mm; mobile phase A: water+0.1% formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nM.

Table 3 lists the structures of the formulae I-3, I-6, I-7, I-8, I-9, I-10 and I-11 of the prior art and indicates which patent discloses the preparation.

TABLE 3

| | | | Analysis |
|---|---|---|---|
| | | | ¹H-NMR: δ [ppm] |
| Ex. | Structure/Name | disclosed in | retention time, [M + H]⁺, Method |
| 3-1 | 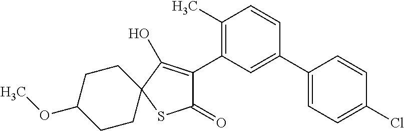 | WO 03/059065 I-3-a-5 | |
| | 3-(4′-chloro-4-methylbiphenyl-3-yl)-4- | | |
| | hydroxy-8-methoxy-1-thiaspiro[4.5]dec- | | |
| | 3-en-2-one | | |
| 3-2 | 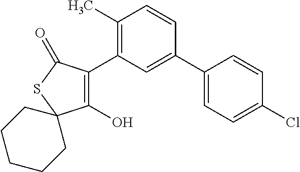 | WO 99/48869 I-3-a-1 | |
| | 3-(4′-chloro-4-methylbiphenyl-3-yl)-4- | | |
| | hydroxy-1-thiaspiro[4.5]dec-3-en-2-one | | |
| 3-3 | 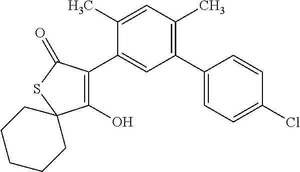 | WO 99/48869 I-3-a-2 | |
| | 3-(4′-chloro-4,6-dimethylbiphenyl-3-yl)- | | |
| | 4-hydroxy-1-thiaspiro[4.5]dec-3-en-2- | | |
| | one | | |
| 3-4 | 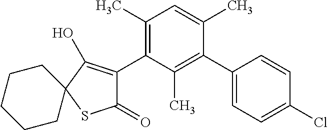 | WO 99/48869 I-3-a-3 | |
| | 3-(4′-chloro-2,4,6-trimethylbiphenyl-3- | | |
| | yl)-4-hydroxy-1-thiaspiro[4.5]dec-3-en- | | |
| | one | | |
| 6-1 | 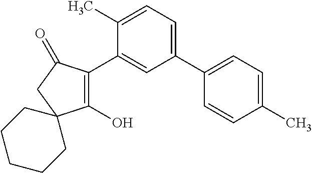 | WO 03/059065 I-4-a-11 | |
| | 3-(4,4′-dimethylbiphenyl-3-yl)-4- | | |
| | hydroxyspiro[4.5]dec-3-en-2-one | | |
| 6-2 | 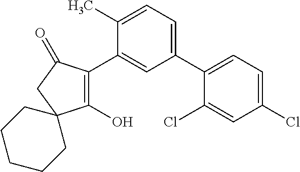 | WO 03/059065 I-4-a-13 | (400 MHz, DMSO-d6): 1.13- 1.26 (m, 1H), 1.27-1.41 (m, 4H), 1.45-1.74 (m, 5H), 2.14 (s, 3H), 2.45-2.55 (m, 2H), 7.05 (d, 1H), 7.23-7.31 (m, 2H), 7.39 (d, 1H), 7.49 (dd, 1H), 7.71 (d, 1H), 12.03 (br. s., 1H). 1.46 min, 401, Method |
| | 3-(2′,4′-dichloro-4-methylbiphenyl-3- | | |
| | yl)-4-hydroxyspiro[4.5]dec-3-en-2-one | | |
| 6-3 | 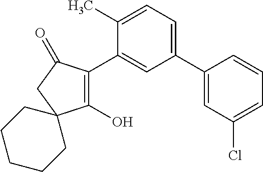 | WO 03/059065 I-4-a-5 | |
| | 3-(3′-chloro-4-methylbiphenyl-3-yl)-4- | | |
| | hydroxyspiro[4.5]dec-3-en-2-one | | |
| 6-4 | 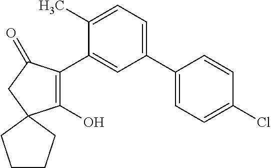 | WO 03/059065 I-4-a-6 | |
| | 3-(4′-chloro-4-methylbiphenyl-3-yl)-4- | | |
| | hydroxyspiro[4.4]non-3-en-2-one | | |
| 6-5 | 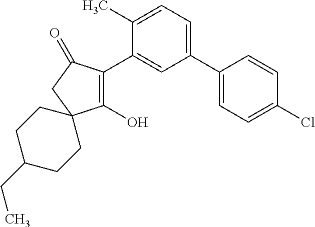 | WO 03/059065 I-4-a-7 | |
| | 3-(4′-chloro-4-methylbiphenyl-3-yl)-8- | | |
| | ethyl-4-hydroxyspiro[4.5]dec-3-en-2- | | |
| | one | | |
| 6-6 | 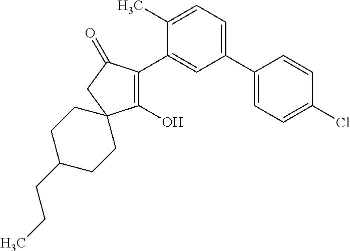 | WO 03/059065 I-4-a-8 | |
| | 3-(4′-chloro-4-methylbiphenyl-3-yl)-4- | | |
| | hydroxy-8-propylspiro[4.5]dec-3-en-2- | | |
| | one | | |
| 6-7 | 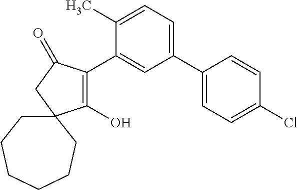 | WO 03/059065 I-4-a-9 | |
| | 3-(4′-chloro-4-methylbiphenyl-3-yl)-4- | | |
| | hydroxyspiro[4.6]undec-3-en-2-one | | |
| 6-8 | 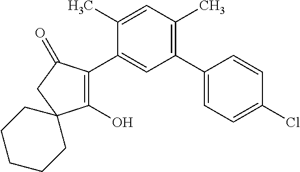 | WO 99/48869 I-7-a-1 | (300 MHz, DMSO-d₆): 1.13- 1.42 (m, 5H), 1.46-1.74 (m, 5H), 2.08 (s, 3H), 2.20 (s, 3H), 2.45-2.55 (m, 2H), 6.82 (s, 1H), 7.12 (s, 1H), 7.30-7.36 (m, 2H), 7.44-7.50 (m, 2H), 11.86 (br. s., 1H). 1.42 min, 381, Method 1 |
| | 3-(4′-chloro-4,6-dimethylbiphenyl-3-yl)- | | |
| | 4-hydroxyspiro[4.5]dec-3-en-2-one | | |
| 6-9 | 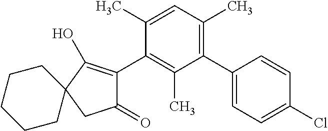 | WO 99/48869 I-7-a-3 | |
| | 3-(4′-chloro-2,4,6-trimethylbiphenyl-3- | | |
| | yl)-4-hydroxyspiro[4.5]dec-3-en-2-one | | |
| 7-1 | 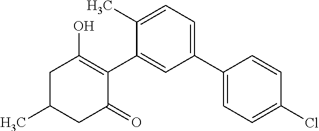 | WO 03/059065 I-5-a-12 | |
| | 2-(4′-chloro-4-methylbiphenyl-3-yl)-3- | | |
| | hydroxy-5-methylcyclohex-2-en-1-one | | |
| 7-2 | 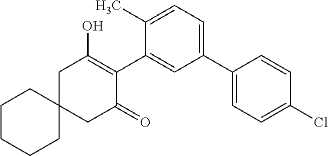 | WO 03/059065 I-5-a-8 | |
| | 3-(4′-chloro-4-methylbiphenyl-3-yl)-4- | | |
| | hydroxyspiro[5.5]undec-3-en-2-one | | |
| 7-3 | 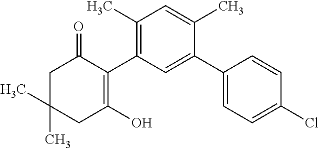 | WO 99/48869 I-8-a-2 | |
| | 2-(4′-chloro-4,6-dimethylbipheny)-3-yl)- | | |
| | 3-hydroxy-5,5-dimethylcyclohex-2-en- | | |
| | 1-one | | |
| 7-4 | 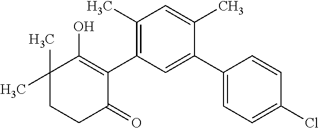 | WO 99/48869 I-8-a-3 | |
| | 2-(4′-chloro-4,6-dimethylbiphenyl-3-yl)- | | |
| | 3-hydroxy-4,4-dimethylcyclohex-2-en- | | |
| | 1-one | | |
| 7-5 | 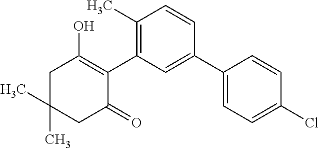 | WO 99/48869 I-8-a-4 | |
| | 2-(4′-chloro-4-methylbiphenyl-3-yl)-3- | | |
| | hydroxy-5,5-dimethylcyclohex-2-en-1- | | |
| | one | | |
| 8-1 | 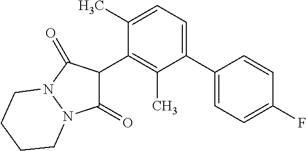 | WO 05/016873 I-1-a-34 | |
| | 2-(4′-fluoro-2,4-dimethylbipheny)-3- | | |
| | yl)tetrahydro-1H-pyrazolo[1,2- | | |
| | a]pyridazine-1,3(2H)-dione | | |
| 8-2 | 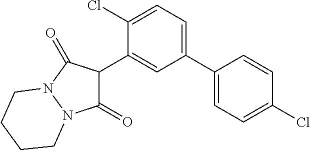 | WO 05/016873 I-1-a-38 | |
| | 2-(4,4′-dichlorobiphenyl-3- | | |
| | yl)tetrahydro-1H-pyrazolo[1,2- | | |
| | a]pyridazine-1,3(2H)-dione | | |
| 8-3 | 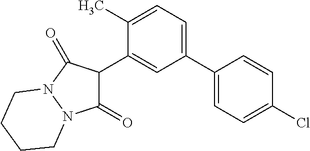 | WO 05/016873 I-1-a-4 | |
| | 2-(4′-chloro-4-methylbiphenyl-3- | | |
| | yl)tetrahydro-1H-pyrazolo[1,2- | | |
| | a]pyridazine-1,3(2H)-dione | | |
| 8-4 | 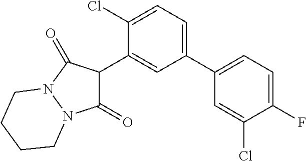 | WO 05/016873 I-1-a-44 | |
| | 2-(3′,4-dichloro-4′-fluorobiphenyl-3- | | |
| | yl)tetrahydro-1H-pyrazolo[1,2- | | |
| | a]pyridazine-1,3(2H)-dione | | |
| 8-5 | 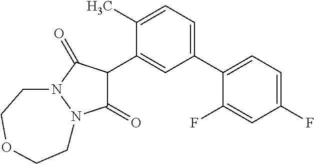 | WO 05/016873 I-2-a-15 | |
| | 8-(2′,4′-difluoro-4-methylbiphenyl-3- | | |
| | yl)tetrahydro-7H-pyrazolo[1,2- | | |
| | d][1,4,5]oxadiazepine-7,9(8H)-dione | | |
| 8-6 | 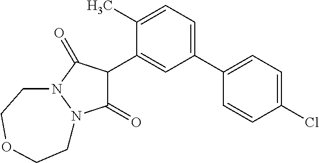 | WO 05/016873 I-2-a-3 | |
| | 8-(4′-chloro-4-methylbiphenyl-3- | | |
| | yl)tetrahydro-7H-pyrazolo[1,2- | | |
| | d][1,4,5]oxadiazepine-7,9(8H)-dione | | |
| 8-7 | 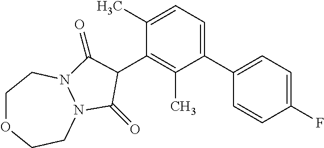 | WO 05/016873 I-2-a-34 | |
| | 8-(4′-fluoro-2,4-dimethylbiphenyl-3- | | |
| | yl)tetrahydro-7H-pyrazolo[1,2- | | |
| | d][1,4,5]oxadiazepine-7,9(8H)-dione | | |
| 8-8 | 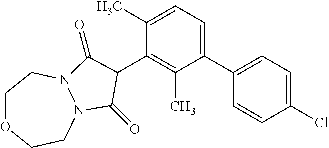 | WO 05/016873 I-2-a-35 | |
| | 8-(4′-chloro-2,4-dimethylbiphenyl-3- | | |
| | yl)tetrahydro-7H-pyrazolo[1,2- | | |
| | d][1,4,5]oxadiazepine-7,9(8H)-dione | | |
| 8-9 | 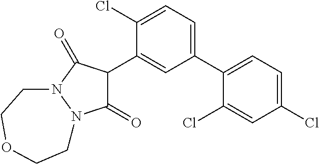 | WO 05/016873 I-2-a-40 | |
| | 8-(2′,4,4′-trichlorobiphenyl-3- | | |
| | yl)tetrahydro-7H-pyrazolo[1,2- | | |
| | d][1,4,5]oxadiazepine-7,9(8H)-dione | | |
| 8-10 | 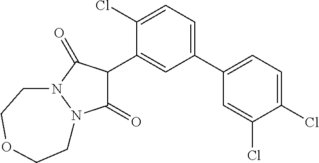 | WO 05/016873 I-2-a-41 | |
| | 8-(3′,4,4′-trichlorobiphenyl-3- | | |
| | yl)tetrahydro-7H-pyrazolo[1,2- | | |
| | d][1,4,5]oxadiazepine-7,9(8H)-dione | | |
| 8-11 | 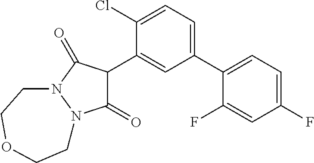 | WO 05/016873 I-2-a-47 | |
| | 8-(4-chloro-2′,4′-difluorobiphenyl-3- | | |
| | yl)tetrahydro-7H-pyrazolo[1,2- | | |
| | d][1,4,5]oxadiazepine-7,9(8H)-dione | | |
| 8-12 | 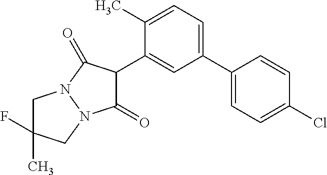 | WO 05/016873 I-5-a-2 | |
| | 2-(4′-chloro-4-methylbiphenyl-3-yl)-6- | | |
| | fluoro-6-methyldihydro-1H,5H- | | |
| | pyrazolo[1,2-a]pyrazole-1,3(2H)-dione | | |
| 8-13 | 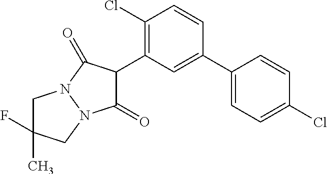 | WO 05/016873 I-5-a-4 | |
| | 2-(4,4′-dichlorobiphenyl-3-yl)-6-fluoro- | | |
| | 6-methyldihydro-1H,5H-pyrazolo [1,2- | | |
| | a]pyrazole-1,3(2H)-dione | | |
| 8-14 | 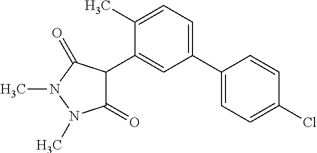 | WO 05/016873 I-6-a-2 | |
| | 4-(4′-chloro-4-methylbiphenyl-3-yl)-1,2- | | |
| | dimethyl-1H-pyrazole-3,5(2H,4H)- | | |
| | dione | | |
| 8-15 | 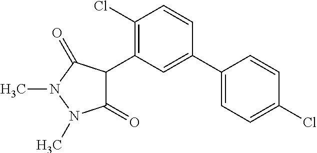 | WO 05/016873 I-6-a-3 | |
| | 4-(4,4′-dichlorobiphenyl-3-yl)-1,2- | | |
| | dimethyl-1H-pyrazole-3,5(2H,4H)- | | |
| | dione | | |
| 8-16 | 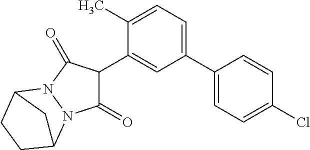 | WO 05/016873 I-8-a-1 | |
| | 2-(4′-chloro-4-methylbiphenyl-3- | | |
| | yl)tetrahydro-1H-5,8- | | |
| | methanopyrazolo[1,2-a]pyridazine- | | |
| | 1,3(2H)-dione | | |
| 8-17 | 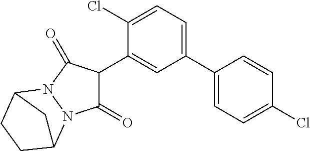 | WO 05/016873 I-8-a-2 | |
| | 2-(4,4′-dichlorobiphenyl-3- | | |
| | yl)tetrahydro-1H-5,8- | | |
| | methanopyrazolo[1,2-a]pyridazine- | | |
| | 1,3(2H)-dione | | |
| 9-1 | 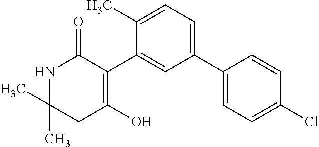 | WO 01/79204 I-a-05 | |
| | 3-(4′-chloro-4-methylbiphenyl-3-yl)-4- | | |
| | hydroxy-6,6-dimethyl-5,6- | | |
| | dihydropyridin-2(1H)-one | | |
| 9-2 | 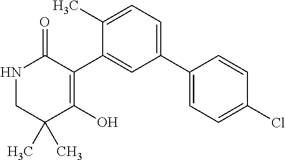 | WO 01/79204 I-a-14 | |
| | 3-(4′-chloro-4-methylbiphenyl-3-yl)-4- | | |
| | hydroxy-5,5-dimethyl-5,6- | | |
| | dihydropyridin-2(1H)-one | | |
| 9-3 | 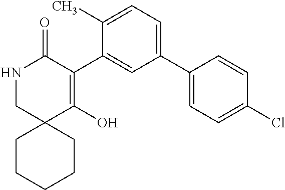 | WO 01/79204 I-a-18 | |
| | 4-(4′-chloro-4-methylbiphenyl-3-yl)-5- | | |
| | hydroxy-2-azaspiro[5.5]undec-4-en-3- | | |
| | one | | |
| 9-4 | 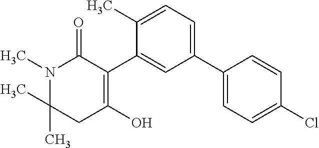 | WO 01/79204 I-a-25 | |
| | 3-(4′-chloro-4-methylbiphenyl-3-yl)-4- | | |
| | hydroxy-1,6,6-trimethyl-5,6- | | |
| 9-5 | 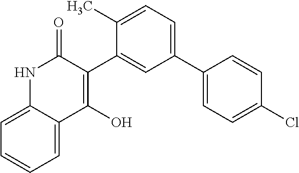 | WO 03/010145 I-a-1 | |
| | 3-(4′-chloro-4-methylbiphenyl-3-yl)-4- | | |
| | hydroxyquinolin-2(1H)-one | | |
| 9-6 | 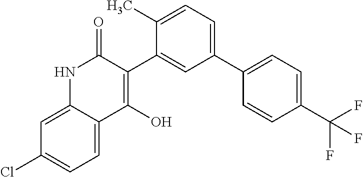 | WO 03/010145 I-a-10 | |
| | 7-chloro-4-hydroxy-3-[4-methyl-4′- | | |
| | (trifluoromethyl)biphenyl-3-yl]quinolin- | | |
| | 2(1H)-one | | |
| 9-7 | 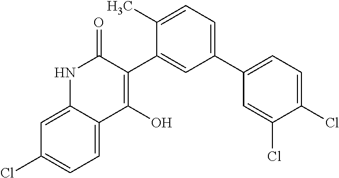 | WO 03/010145 I-a-12 | |
| | 7-chloro-3-(3′,4′-dichloro-4-methyl- | | |
| | biphenyl-3-yl)-4-hydroxyquinolin- | | |
| | 2(1H)-one | | |
| 9-8 | 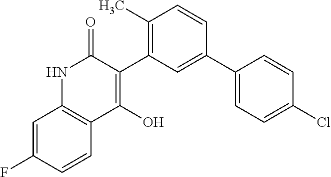 | WO 03/010145 I-a-13 | |
| | 3-(4′-chloro-4-methylbiphenyl-3-yl)-7- | | |
| | fluoro-4-hydroxyquinolin-2(1H)-one | | |
| 9-9 | 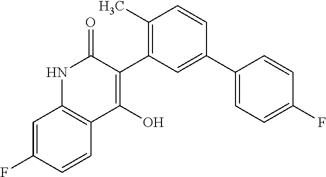 | WO 03/010145 I-a-18 | |
| | 7-fluoro-3-(4′-fluoro-4-methylbiphenyl- | | |
| | 3-yl)-4-hydroxyquinolin-2(1H)-one | | |
| 9-10 | 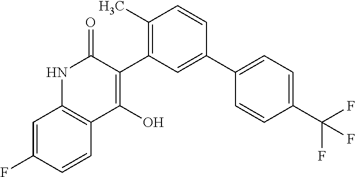 | WO 03/010145 I-a-19 | |
| | 7-fluoro-4-hydroxy-3-[4-methyl-4′- | | |
| | (trifluoromethyl)biphenyl-3-yl]quinolin- | | |
| | 2(1H)-one | | |
| 9-11 | 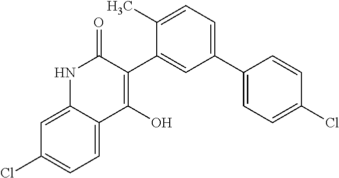 | WO 03/010145 I-a-3 | (300 MHz, DMSO-d6): 2.13 (s, 3H), 7.21 (dd, 1H), 7.32-7.43 (m, 3H), 7.45-7.52 (m, 2H), 7.57 (dd, 1H), 7.64-7.72 (m, 2H), 7.94 (d, 1H), 10.33 (br. s., 1H), 11.53 (s, 1H). 1.34 min, 396, Method 1 |
| | 7-chloro-3-(4′-chloro-4-methylbiphenyl- | | |
| | 3-yl)-4-hydroxyquinolin-2(1H)-one | | |
| 9-12 | 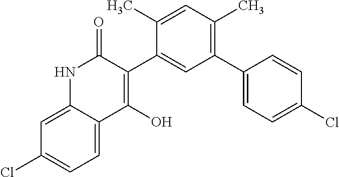 | WO 03/010145 I-a-7 | |
| | 7-chloro-3-(4′-chloro-4,6-dimethyl- | | |
| | biphenyl-3-yl)-4-hydroxyquinolin- | | |
| | 2(1H)-one | | |
| 9-13 | 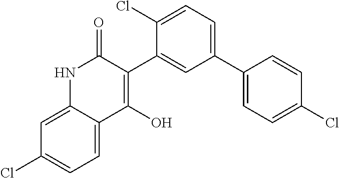 | WO 03/010145 I-a-8 | |
| | 7-chloro-3-(4,4′-dichlorobiphenyl-3-yl)- | | |
| | 4-hydroxyquinolin-2(1H)-one | | |
| 9-14 | 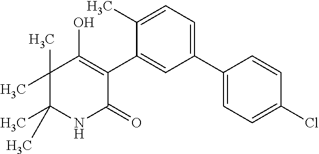 | WO 07/068428 I-9-a-1 | |
| | 3-(4′-chloro-4-methylbiphenyl-3-yl)-4- | | |
| | hydroxy-5,5,6,6-tetramethyl-5,6- | | |
| | dihydropyridin-2(1H)-one | | |
| 10-1 | 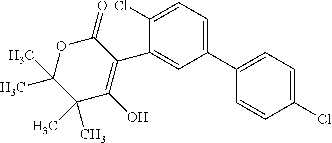 | WO 01/98288 I-a-31 | |
| | 3-(4,4′-dichlorobiphenyl-3-yl)-4- | | |
| | hydroxy-5,5,6,6-tetramethyl-5,6- | | |
| | dihydro-2H-pyran-2-one | | |
| 10-2 | 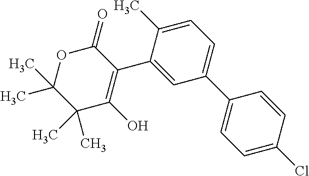 | WO 01/98288 I-a-6 | |
| | 3-(4′-chloro-4-methylbiphenyl-3-yl)-4- | | |
| | hydroxy-5,5,6,6-tetramethyl-5,6- | | |
| | dihydro-2H-pyran-2-one | | |
| 11-1 | 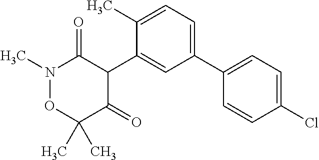 | WO 03/048138 I-a-7 | |
| | 4-(4′-chloro-4-methylbiphenyl-3-yl)- | | |
| | 2,6,6-trimethyl-1,2-oxazinane-3,5-dione | | |

### Method 1 (UPLC-MS):

instrument: Waters Acquity UPLC-MS SQD 3001; column: Acquity UPLC BEH C18 1.7 50×2.1 mm; mobile phase A: water+0.1% formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nM.

### Method 2 (UPLC-MS):

instrument: Waters Acquity UPLC-MS ZQ4000; column: Acquity UPLC BEH C18 1.7 50×2.1 mm; mobile phase A: water+0.05% formic acid, mobile phase B: acetonitrile+0.05% formic acid; gradient: 0-1.6 min 1-99% B, 1.6-2.0 min 99% B; flow rate 0.8 ml/min; temperature: 60° C.; injection: 2 μl; DAD scan: 210-400 nM.

### Assays

### Human ACC1 Enzyme Assay

The inhibitory activity of the substances of this invention with regard to acetyl-CoA carboxylase 1 (ACC1) was measured using the ACC1 assay described in the paragraphs below. The basic principle of the assay is the measurement of adenosine diphosphate (ADP), which is formed as a by-product, by means of an HTRF®-based competitive immunoassay (HTRF=Homogeneous Time Resolved Fluorescence).

The enzyme used was C-terminally FLAG-tagged recombinant human ACC1 (GenBank Accession no. NM_{—}198834, amino acids 39—end), expressed in baculovirus-transfected insect cells (Hi5) and purified by affinity chromatography on Anti-FLAG®M2 affinity gel (Sigma-Aldrich). Alternatively, it is possible to use commercial C-terminally His-tagged ACC1 from BPS Bioscience (San Diego, Calif., catalogue no. 50200, amino acids 39—end). For the assay, 50 n1 of a 100-fold concentrated solution of the test substance in DMSO were pipetted into a black low-volume 384-well microtitre plate (Greiner Bio-One, Frickenhausen, Germany), 2 μl of a solution of ACC1 in assay buffer [50 mM HEPES/NaOH pH 7.5, 12 mM sodium bicarbonate, 2 mM MgCl₂, 2 mM potassium citrate, 0.005% (w/v) bovine serum albumin (BSA)] were added and the mixture was incubated for 15 min to allow pre-binding of the substances to the enzyme prior to the enzyme reaction. The enzyme reaction was then started by addition of 3 μl of a solution of adenosine triphosphate (ATP, 83.5 μM=>the final concentration in 5 μl assay volume is 50 μM, Amersham Pharmacia Biotech #27-2056-01) and acetyl-CoA (33.4 μM=>the final concentration in 5 μl assay volume is 20 μM, Roche Bioscience #10101893001) in assay buffer, and the resulting mixture was incubated at 22° C. for a reaction time of 20 min. The concentration of the ACC1 was adjusted to the respective activity of the enzyme and set such that the assay was carried out in the linear range. Typical concentrations were in the range of 2.5 ng/μl.

The reaction was stopped by successive addition of 2.5 μl of a solution of d2-labelled ADP (HTRF® Transscreener™ ADP kit, C is biointernational, Marcoule, France) in EDTA-containing HTRF® Transscreener™ ADP detection buffer (contained in the HTRF® Transscreener™ ADP kit, 50 mM HEPES pH 7.0, 60 mM EDTA, 0.1% (w/v) BSA, 0.02% sodium azide, 400 mM potassium fluoride) and 2.5 μl of a solution of europium cryptate-labelled anti-ADP antibody (HTRF® Transscreener™ ADP kit) in HTRF® Transscreener™ ADP detection buffer.

The resulting mixture was incubated at 22° C. for 1 h to allow binding of the europium cryptate-labelled anti-ADP antibody to the ADP formed by the enzyme reaction and the d2-labelled ADP. The amount of complex of d2-labelled ADP and europium cryptate-labelled anti-ADP antibody was then determined by measuring the resonance energy transfer of europium cryptate to d2. To this end, the fluorescence emissions at 620 nm and 665 nm after excitation at 350 nm were measured in an HTRF measuring instrument, for example a Rubystar or Pherastar (both BMG Labtechnologies, Offenburg, Germany). The ratio of the emissions at 665 nm and at 622 nm was taken as a measure of the amount of the complex of d2-labelled ADP and europium cryptate-labelled anti-ADP antibody and thus indirectly as a measure for the amount of unlabelled ADP formed in the enzyme reaction (higher ratio of the emissions at 665 nm and at 622 nm [symbol] more complex of d2-labelled ADP and europium cryptate-labelled anti-ADP antibody [symbol] less ADP). The data were normalized (enzyme reaction without inhibitor=0% inhibition, all other assay components but no enzyme=100% inhibition). The test substances were usually tested on the same microtitre plates at 10 different concentrations in the range from 20 μM to 1 nM (20 μM, 6.7 μM, 2.2 μM, 0.74 μM, 0.25 μM, 82 nM, 27 nM, 9.2 nM, 3.1 nM and 1 nM, the dilution series were prepared prior to the assay based on the 100-times concentrated solution by serial 1:3 dilutions) in two replications for each concentration, and IC50 values were calculated with a 4-parameter fit using an inhouse software.

### Human ACC2 Enzyme Assay

The inhibitory activity of the substances of this invention with regard to acetyl-CoA carboxylase 2 (ACC2) was measured using the ACC2 assay described in the paragraphs below. The basic principle of the assay is the measurement of adenosine diphosphate (ADP), which is formed as a by-product, by means of an HTRF®-based competitive immunoassay (HTRF=Homogeneous Time Resolved Fluorescence).

The enzyme used was commercially available C-terminally His-tagged ACC2 from BPS Bioscience (San Diego, Calif., catalogue no. 50201, amino acids 39—end, expressed in baculovirus-transfected Sf9 insect cells and purified by Ni-NTA affinity chromatography).

For the assay, 50 n1 of a 100-fold concentrated solution of the test substance in DMSO were pipetted into a black low-volume 384-well microtitre plate (Greiner Bio-One, Frickenhausen, Germany), 2 μl of a solution of ACC2 in assay buffer [50 mM HEPES/NaOH pH 7.5, 12 mM sodium bicarbonate, 2 mM MgCl₂, 2 mM potassium citrate, 0.005% (w/v) bovine serum albumin (BSA)] were added and the mixture was incubated for 15 min to allow pre-binding of the substances to the enzyme prior to the enzyme reaction. The enzyme reaction was then started by addition of 3 μl of a solution of adenosine triphosphate (ATP, 83.5 μM=>the final concentration in 5 μl assay volume is 50 Amersham Pharmacia Biotech #27-2056-01) and acetyl-CoA (33.4 μM=>the final concentration in 5 μl assay volume is 20 μM, Roche Bioscience #10101893001) in assay buffer, and the resulting mixture was incubated at 22° C. for a reaction time of 20 min. The concentration of the ACC2 was adjusted to the respective activity of the enzyme and set such that the assay was carried out in the linear range. Typical concentrations were in the range of 0.6 ng/μl.

The reaction was stopped by successive addition of 2.5 μl of a solution of d2-labelled ADP (HTRF® Transscreener™ ADP kit, C is biointernational, Marcoule, France) in EDTA-containing HTRF® Transscreener™ ADP detection buffer (contained in the HTRF® Transscreener™ ADP kit, 50 mM HEPES pH 7.0, 60 mM EDTA, 0.1% (w/v) BSA, 0.02% sodium azide, 400 mM potassium fluoride) and 2.5 μl of a solution of europium cryptate-labelled anti-ADP antibody (HTRF® Transscreener™ ADP kit) in HTRF® Transscreener™ ADP detection buffer.

The resulting mixture was incubated at 22° C. for 1 h to allow binding of the europium cryptate-labelled anti-ADP antibody to the ADP formed by the enzyme reaction and the d2-labelled ADP. The amount of complex of d2-labelled ADP and europium cryptate-labelled anti-ADP antibody was then determined by measuring the resonance energy transfer of europium cryptate to d2. To this end, the fluorescence emissions at 620 nm and 665 nm after excitation at 350 nm were measured in an HTRF measuring instrument, for example a Rubystar or Pherastar (both BMG Labtechnologies, Offenburg, Germany). The ratio of the emissions at 665 nm and at 622 nm was taken as a measure of the amount of the complex of d2-labelled ADP and europium cryptate-labelled anti-ADP antibody and thus indirectly as a measure for the amount of unlabelled ADP formed in the enzyme reaction (higher ratio of the emissions at 665 nm and at 622 nm [symbol] more complex of d2-labelled ADP and europium cryptate-labelled anti-ADP antibody [symbol] less ADP). The data were normalized (enzyme reaction without inhibitor=0% inhibition, all other assay components but no enzyme=100% inhibition). The test substances were usually tested on the same microtitre plates at 10 different concentrations in the range from 20 μM to 1 nM (20 μM, 6.7 μM, 2.2 μM, 0.74 μM, 0.25 μM, 82 nM, 27 nM, 9.2 nM, 3.1 nM and 1 nM, the dilution series were prepared prior to the assay based on the 100-times concentrated solution by serial 1:3 dilutions) in two replications for each concentration, and 1050 values were calculated with a 4-parameter fit using an inhouse software.

### Non-Human ACCase Assay

The assay was carried out at room temperature in a transparent 384-well microtitre plate. It determined the inorganic phosphate released from the ATP in the ACCase reaction.

The test mixture contained 50 mM Tris-HCl pH 8.3, 50 mM KCl, 2.5 mM MgCl₂, 0.5 mM ATP, 0.8 mM dithiothreitol (DTT), 30 mM NaHCO₃, 0.1 mM acetyl-CoA, 0.04% bovine serum albumin and 0.4 μg partially purified ACCase enzyme in a final volume of 40 μl. After 45 minutes of incubation, the reaction was stopped with 150 μl of malachite green solution, and the absorption at 620 was read after 30 minutes.

The malachite green (MG) solution was prepared by mixing 3 parts of 0.6 mM MG-HCl solution in distilled water with 1 part of 8.5 mM ammonium molybdate in 4 M HCl. The solution was allowed to stand for 30 minutes. After filtration through a 0.45 μm polytetrafluoroethylene (PTFE) filter, 0.1 part of Triton X-100 (1.5%) in distilled water was added.

ACCase enzyme was extracted from oat seedlings 9 days after sowing and partially purified by precipitation with 0-40% ammonium sulphate followed by ion exchange chromatography on Q-Sepharose.

### Results:

Table 4 summarizes the results of the enzyme assays and the mode-of-action test for the compounds of the formula I-1.

TABLE 4

| | ACC 1 | ACC 2 |
|---|---|---|
| Example No. | IC50 [μmol/l] | IC50 [μmol/l] |
| 1-1 | 0.46 | 8.8 |
| 1-2 | 0.28 | 0.37 |
| 1-3 | 0.45 | 5.4 |
| 1-4 | 0.46 | 0.33 |
| 1-5 | 0.287 | 8.242 |
| 1-6 | 0.30 | 5.2 |
| 1-7 | 1.7 | 0.087 |
| 1-8 | 0.79 | 0.81 |
| 1-9 | 16.605 | 0.626 |
| 1-10 | 0.550 | 6.402 |
| 1-11 | 0.24 | 1.0 |
| 1-12 | 0.772 | 18.219 |
| 1-13 | 0.353 | 18.936 |
| 1-14 | 0.990 | >20 |
| 1-15 | 0.742 | >20 |
| 1-16 | 0.329 | >20 |
| 1-17 | 0.334 | 4.021 |
| 1-18 | 0.797 | 7.608 |
| 1-19 | 0.874 | 6.965 |
| 1-20 | 0.240 | 0.214 |
| 1-21 | 0.533 | 15.131 |
| 1-22 | 0.969 | 0.695 |
| 1-23 | 0.584 | 0.485 |
| 1-24 | 0.34 | 0.21 |
| 1-25 | 0.495 | 3.356 |
| 1-26 | 0.540 | 1.254 |
| 1-27 | 0.472 | >20 |
| 1-28 | 0.443 | 0.192 |
| 1-29 | 0.27 | 1.9 |
| 1-30 | 0.542 | 0.300 |
| 1-31 | 0.963 | 5.956 |
| 1-32 | 0.86 | 14 |
| 1-33 | 0.359 | 0.535 |
| 1-34 | 0.47 | 9.4 |
| 1-35 | 0.298 | 3.998 |
| 1-36 | 0.792 | >20 |
| 1-37 | 0.810 | >20 |
| 1-38 | 0.497 | >20 |
| 1-39 | 0.663 | >20 |
| 1-40 | 1.2 | 15 |
| 1-41 | 0.751 | >20 |
| 1-42 | >20 | 0.669 |
| 1-43 | 0.304 | 10.240 |
| 1-44 | 0.915 | >20 |
| 1-45 | 0.776 | 3.608 |
| 1-46 | 0.812 | 15.779 |
| 1-47 | 0.923 | >20 |
| 1-48 | 1.006 | 8.598 |
| 1-49 | 0.24 | 9.9 |
| 1-50 | 0.472 | 6.267 |
| 1-51 | 0.473 | 4.913 |
| 1-52 | 1.268 | >20 |
| 1-53 | 0.506 | 0.214 |
| 1-54 | 0.22 | 1.1 |
| 1-55 | 0.918 | 0.465 |
| 1-56 | 0.43 | 0.32 |
| 1-57 | 0.79 | 0.22 |
| 1-58 | 0.362 | 11.486 |
| 1-59 | 0.427 | 5.205 |
| 1-60 | 0.237 | 2.334 |
| 1-61 | 0.227 | 3.910 |
| 1-62 | 0.690 | |
| 1-63 | 0.391 | 4.216 |
| 1-64 | 0.985 | 16.443 |
| 1-65 | 0.934 | 9.465 |
| 1-66 | 0.409 | 1.542 |
| 1-67 | 0.668 | 1.443 |
| 1-68 | 0.489 | 4.860 |
| 1-69 | 0.647 | 10.997 |
| 1-70 | 0.675 | 1.839 |
| 1-71 | 0.661 | >20 |
| 1-72 | 1.179 | 2.340 |
| 1-73 | 0.912 | 4.971 |
| 1-74 | 0.656 | 16.428 |
| 1-75 | 7.975 | 0.376 |
| 1-76 | 0.201 | 0.233 |
| 1-77 | 0.327 | 0.860 |
| 1-78 | 0.795 | 1.729 |
| 1-79 | 0.848 | >20 |
| 1-80 | 0.695 | >20 |
| 1-81 | 0.327 | 1.414 |
| 1-82 | 0.542 | 0.433 |
| 1-83 | 0.459 | 0.349 |
| 1-84 | 0.455 | |
| 1-85 | 0.34 | 4.0 |
| 1-86 | 0.541 | 3.302 |
| 1-87 | 0.54 | 7.3 |
| 1-88 | 0.825 | 6.898 |
| 1-89 | 0.858 | >20 |

Table 5 summarizes the results of the enzyme assays and the mode-of-action test for the compounds of the formula I-2.

TABLE 5

| | ACC 1 | ACC 2 |
|---|---|---|
| Example No. | IC50 [μmol/l] | IC50 [μmol/l] |
| 2-1 | 0.243 | 4.150 |
| 2-2 | 0.260 | 5.285 |
| 2-3 | 0.581 | 11.696 |
| 2-4 | 0.19 | 1.5 |
| 2-5 | 1.478 | 5.715 |
| 2-6 | 2.151 | 0.139 |
| 2-7 | 0.500 | >20 |
| 2-8 | 0.420 | >20 |
| 2-9 | 0.334 | >20 |
| 2-10 | 0.161 | 2.354 |
| 2-11 | 0.666 | 1.547 |
| 2-12 | 4.173 | 1.152 |
| 2-13 | 0.816 | >20 |
| 2-14 | 0.562 | 19.353 |
| 2-15 | 0.204 | 3.676 |
| 2-16 | 0.180 | 3.336 |
| 2-17 | 0.158 | 0.652 |
| 2-18 | 0.479 | 3.409 |
| 2-19 | 0.530 | 10.007 |
| 2-20 | 0.137 | 1.929 |
| 2-21 | 0.40 | >20 |
| 2-22 | 0.824 | >20 |
| 2-23 | 1.369 | >20 |
| 2-24 | 0.997 | >20 |
| 2-25 | 0.442 | >20 |
| 2-26 | 0.26 | 4.8 |
| 2-27 | 0.822 | 4.146 |
| 2-28 | 0.14 | 2.5 |
| 2-29 | 0.321 | 0.789 |
| 2-30 | 0.281 | 0.032 |
| 2-31 | 0.88 | 0.048 |
| 2-32 | 0.45 | 1.6 |
| 2-33 | 0.229 | 3.586 |
| 2-34 | 0.286 | 1.274 |
| 2-35 | 1.024 | >20 |
| 2-36 | n.d. | 1.936 |
| 2-37 | 0.342 | >20 |
| 2-38 | 0.416 | 3.785 |
| 2-39 | 0.216 | 3.533 |
| 2-40 | 1.216 | >20 |
| 2-41 | 0.791 | 4.217 |
| 2-42 | 0.357 | 1.021 |
| 2-43 | 0.504 | 0.330 |
| 2-44 | 0.297 | 0.160 |
| 2-45 | 0.568 | 17 |
| 2-46 | 0.580 | 7.610 |
| 2-48 | 1.800 | 0.204 |
| 2-49 | 5.776 | 1.281 |
| 2-50 | 0.903 | 8.593 |

Table 6 summarizes the results of the enzyme assays for the compounds of the formulae I-3, I-6, I-7, I-8, I-9, I-10 and I-11.

TABLE 6

| | ACC 1 | ACC 2 |
|---|---|---|
| Example No. | IC50 [μmol/l] | IC50 [μmol/l] |
| 3-1 | 0.853 | |
| 3-2 | 0.097 | 1.675 |
| 3-3 | 0.34 | 0.25 |
| 3-4 | 1.311 | 0.228 |
| 6-1 | 0.655 | 16.024 |
| 6-2 | 0.337 | 1.688 |
| 6-3 | 1.390 | 16.220 |
| 6-4 | 0.333 | 3.059 |
| 6-5 | 0.754 | 0.565 |
| 6-6 | 0.923 | 0.783 |
| 6-7 | 0.467 | 0.654 |
| 6-8 | 1.379 | 0.127 |
| 6-9 | >20 | 0.387 |
| 7-1 | 0.678 | >20 |
| 7-2 | 0.578 | >20 |
| 7-3 | 1.589 | 5.337 |
| 7-4 | 3.673 | 3.711 |
| 7-5 | 0.610 | >20 |
| 8-1 | 2.459 | >20 |
| 8-2 | 0.891 | >20 |
| 8-3 | 1.142 | >20 |
| 8-4 | 3.180 | >20 |
| 8-5 | 2.076 | >20 |
| 8-6 | 1.382 | >20 |
| 8-7 | 2.381 | >20 |
| 8-8 | 0.936 | 16.858 |
| 8-9 | 0.457 | >20 |
| 8-10 | 1.126 | >20 |
| 8-11 | 0.713 | >20 |
| 8-12 | 0.587 | >20 |
| 8-13 | 0.640 | >20 |
| 8-14 | 2.399 | >20 |
| 8-15 | 1.966 | >20 |
| 8-16 | 0.662 | >20 |
| 8-17 | 0.913 | >20 |
| 9-1 | 0.319 | >20 |
| 9-2 | 0.806 | 9.080 |
| 9-3 | 1.063 | 4.281 |
| 9-4 | 5.379 | >20 |
| 9-5 | 0.625 | >20 |
| 9-6 | 1.092 | 18.943 |
| 9-7 | 0.000 | 8.289 |
| 9-8 | 0.418 | >20 |
| 9-9 | 0.863 | >20 |
| 9-10 | 1.693 | >20 |
| 9-11 | 0.420 | 4.117 |
| 9-12 | 0.580 | |
| 9-13 | 0.329 | 5.557 |
| 9-14 | 1.639 | 5.622 |
| 10-1 | 1.367 | >20 |
| 10-2 | 0.586 | >20 |
| 11-1 | 3.996 | >20 |

Table C.2 summarizes the results of the enzyme assays for the comparative compounds.

TABLE C.2

| Example | ACC 1 | ACC 2 | ACC-non human |
|---|---|---|---|
| No. | IC50 [μmol/l] | IC50 [μmol/l] | IC50 [μmol/l] |
| C-1 | >20 | >20 | 0.0005 |
| C-2 | >20 | >20 | 0.04 |
| C-3 | >20 | >20 | 0.002 |
| C-4 | 5.4 | >20 | 0.02 |

The results show very clearly that, in spite of close structural similarity, it is not possible to predict whether structures inhibiting non-human ACC are also inhibitors of human ACCs.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of U.S. Provisional Application Ser. No. 61/375,375, filed Aug. 20, 2010, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. (5s,8s)-3-(4,4′-dichlorobiphenyl-3-yl)-4-hydroxy-8-(2,2,2-trifluoroethoxy)-1-azaspiro[4.5]dec-3-en-2-one,
(5s,8s)-3-(4′-chloro-2,4-dimethylbiphenyl-3-yl)-4-hydroxy-8-(2,2,2-trifluoroethoxy)-1-azaspiro[4.5]dec-3-en-2-one,
3-(4′-fluoro-2,4-dimethylbiphenyl-3-yl)-4-hydroxy-8-(2,2,2-trifluoroethoxy)-1-azaspiro[4.5]dec-3-en-2-one,
3-(4′-fluoro-4-methylbiphenyl-3-yl)-4-hydroxy-8-(2,2,2-trifluoroethoxy)-1-azaspiro[4.5]dec-3-en-2-one,
3-(4′-chloro-4-methylbiphenyl-3-yl)-4-hydroxy-8-(2,2,2-trifluoroethoxy)-1-azaspiro[4.5]dec-3-en-2-one,
11-(4′-chloro-2,4-dimethylbiphenyl-3-yl)-12-hydroxy-1-oxa-9-azadispiro[4.2.4.2]tetradec-11-en-10-one,
11-(3′,4′-difluoro-4-methylbiphenyl-3-yl)-12-hydroxy-1-oxa-9-azadispiro[4.2.4.2]tetradec-11-en-10-one,
4-hydroxy-3-(3′,4′,5′-trifluoro-4-methylbiphenyl-3-yl)-8-oxa-1-azaspiro[4.5]dec-3-en-2-one,
3-(3′,4′-difluoro-4-methylbiphenyl-3-yl)-4-hydroxy-8-oxa-1-azaspiro[4.5]dec-3-en-2-one,
3-(4-chloro-3′,4′,5′-trifluorobiphenyl-3-yl)-4-hydroxy-8-oxa-1-azaspiro[4.5]dec-3-en-2-one,
3-(4-chloro-3′-fluoro-4′-methylbiphenyl-3-yl)-4-hydroxy-8-oxa-1-azaspiro[4.5]dec-3-en-2-one,
3-(4′-fluoro-2,4-dimethylbiphenyl-3-yl)-4-hydroxy-1-azaspiro[4.5]dec-3-en-2-one,
3-(4-chloro-4′-fluorobiphenyl-3-yl)-4-hydroxy-8-oxa-1-azaspiro[4.5]dec-3-en-2-one,
(5s,8s)-3-(4′-fluoro-2,4,6-trimethylbiphenyl-3-yl)-4-hydroxy-8-methoxy-1-azaspiro[4.5]dec-3-en-2-one,
(5s,8s)-3-(3′-chloro-4′-fluoro-4-methylbiphenyl-3-yl)-4-hydroxy-8-methoxy-1-azaspiro[4.5]dec-3-en-2-one
(5s,8s)-3-(4-chloro-4′-fluorobiphenyl-3-yl)-4-hydroxy-8-methoxy-1-azaspiro[4.5]dec-3-en-2-one,
(5s,8s)-3-(4′-fluoro-2,4-dimethylbiphenyl-3-yl)-4-hydroxy-8-methoxy-1-azaspiro[4.5]dec-3-en-2-one,
3-(4′-chloro-4-methylbiphenyl-3-yl)-4-hydroxy-5-(methoxymethyl)-5-methyl-1,5-dihydro-2H-pyrrol-2-one,
3-(4′-chloro-4-methylbiphenyl-3-yl)-4-hydroxy-5-(2-methoxyethyl)-5-methyl-1,5-dihydro-2H-pyrrol-2-one,
3-(4′-chloro-2,4-dimethylbiphenyl-3-yl)-4-hydroxy-7-methoxy-1-azaspiro[4.4]non-3-en-2-one,
rel-(5R,7R)-3-(4′-chloro-4-methylbiphenyl-3-yl)-4-hydroxy-7-(2-methylpropoxy)-1-azaspiro[4.4]non-3-en-2-one,
rel-(5R,7S)-3-(4′-chloro-4-methylbiphenyl-3-yl)-7-(2-ethoxyethoxy)-4-hydroxy-1-azaspiro[4.4]non-3-en-2-one,
3-(4′-chloro-4-methylbiphenyl-3-yl)-4-hydroxy-7-(methoxymethyl)-1-azaspiro[4.5]dec-3-en-2-one,
3-(4,4′-dichlorobiphenyl-3-yl)-4-hydroxy-7-(methoxymethyl)-1-azaspiro[4.5]dec-3-en-2-one,
3-(4′-chloro-2,4-dimethylbiphenyl-3-yl)-4-hydroxy-8-(methoxymethyl)-1-azaspiro[4.5]dec-3-en-2-one,
3-(4,4′-dichlorobiphenyl-3-yl)-4-hydroxy-8-(methoxymethyl)-1-azaspiro[4.5]dec-3-en-2-one,
3-(4′-chloro-2,4-dimethylbiphenyl-3-yl)-4-hydroxy-7-(2-methoxyethyl)-1-azaspiro[4.5]dec-3-en-2-one,
3-(4′-chloro-4-methylbiphenyl-3-yl)-4-hydroxy-8-(methoxymethyl)-1-azaspiro[4.5]dec-3-en-2-one,
3-(4′-chloro-2,4-dimethylbiphenyl-3-yl)-4-hydroxy-8-(2-methoxyethyl)-1-azaspiro[4.5]dec-3-en-2-one,
3-(4,4′-dichlorobiphenyl-3-yl)-4-hydroxy-9,13-dioxa-1-azadispiro[4.2.5.2]pentadec-3-en-2-one,
3-(4′-chloro-4-methylbiphenyl-3-yl)-4-hydroxy-9,13-dioxa-1-azadispiro[4.2.5.2]pentadec-3-en-2-one,
11-(4,4′-dichlorobiphenyl-3-yl)-12-hydroxy-1,4-dioxa-9-azadispiro[4.2.4.2]tetradec-11-en-10-one,
11-(4′-chloro-2,4-dimethylbiphenyl-3-yl)-12-hydroxy-2-methyl-1,4-dioxa-9-azadispiro[4.2.4.2]tetradec-11-en-10-one,
11-(4′-chloro-4-methylbiphenyl-3-yl)-12-hydroxy-2-methyl-1,4-dioxa-9-azadispiro[4.2.4.2]tetradec-11-en-10-one,
11-(4′-chloro-4-methylbiphenyl-3-yl)-12-hydroxy-2,3-dimethyl-1,4-dioxa-9-azadispiro[4.2.4.2]tetradec-11-en-10-one,
3-(4,4′-dichlorobiphenyl-3-yl)-4-hydroxy-11,11-dimethyl-9,13-dioxa-1-azadispiro[4.2.5.2]pentadec-3-en-2-one,
3-(4′-chloro-4-methylbiphenyl-3-yl)-4-hydroxy-1′-methyl-9,13-dioxa-1-azadispiro[4.2.5.2]pentadec-3-en-2-one,
3-(4′-chloro-4-methylbiphenyl-3-yl)-4-hydroxy-11,11-dimethyl-9,13-dioxa-1-azadispiro[4.2.5.2]pentadec-3-en-2-one,
11-(4,4′-dichlorobiphenyl-3-yl)-12-hydroxy-2-methyl-1,4-dioxa-9-azadispiro[4.2.4.2]tetradec-1′-en-10-one,
3-(4,4′-dichlorobiphenyl-3-yl)-4-hydroxy-1′-methyl-9,13-dioxa-1-azadispiro[4.2.5.2]pentadec-3-en-2-one,
11-(4′-chloro-2,4-dimethylbiphenyl-3-yl)-12-hydroxy-1,4-dioxa-9-azadispiro[4.2.4.2]tetradec-1′-en-10-one,
11-(4,4′-dichlorobiphenyl-3-yl)-12-hydroxy-2,3-dimethyl-1,4-dioxa-9-azadispiro[4.2.4.2]tetradec-11-en-10-one,
3-(4′-chloro-2,4-dimethylbiphenyl-3-yl)-4-hydroxy-7-methoxy-1-azaspiro[4.5]dec-3-en-2-one,
3-(4′-chloro-2,4-dimethylbiphenyl-3-yl)-7-ethoxy-4-hydroxy-1-azaspiro[4.5]dec-3-en-2-one,
rel-(5R,7R)-3-(4,4′-dichlorobiphenyl-3-yl)-4-hydroxy-7-(2-methoxyethoxy)-1-azaspiro[4.5]dec-3-en-2-one,
3-(4′-chloro-2,4,6-trimethylbiphenyl-3-yl)-8-ethoxy-4-hydroxy-1-azaspiro[4.5]dec-3-en-2-one,
(5r,8r)-3-(4′-chloro-2,4-dimethylbiphenyl-3-yl)-4-hydroxy-8-methoxy-8-methyl-1-azaspiro[4.5]dec-3-en-2-one,
(5r,8r)-3-(4′-chloro-2,4-dimethylbiphenyl-3-yl)-8-ethyl-4-hydroxy-8-methoxy-1-azaspiro[4.5]dec-3-en-2-one,
(5r,8r)-8-ethyl-3-(4′-fluoro-2,4-dimethylbiphenyl-3-yl)-4-hydroxy-8-methoxy-1-azaspiro[4.5]dec-3-en-2-one,
3-(4′-chloro-4,6-dimethylbiphenyl-3-yl)-4-hydroxy-7-methoxy-1-oxaspiro[4.5]dec-3-en-2-one,
3-(4′-chloro-2,4,6-trimethylbiphenyl-3-yl)-4-hydroxy-7-methoxy-1-oxaspiro[4.5]dec-3-en-2-one,
3-(4,4′-dichlorobiphenyl-3-yl)-4-hydroxy-7-methoxy-1-oxaspiro[4.5]dec-3-en-2-one,
3-(4′-chloro-4-methylbiphenyl-3-yl)-4-hydroxy-7-methoxy-1-oxaspiro[4.5]dec-3-en-2-one,
11-(4′-chloro-4-methylbiphenyl-3-yl)-12-hydroxy-1,4,9-trioxadispiro[4.2.4.2]tetradec-11-en-10-one,
3-(4,4′-dichlorobiphenyl-3-yl)-4-hydroxy-8-(methoxymethyl)-1-oxaspiro[4.5]dec-3-en-2-one,
3-(4′-chloro-2,4-dimethylbiphenyl-3-yl)-4-hydroxy-8-(methoxymethyl)-1-oxaspiro[4.5]dec-3-en-2-one,
3-(4′-chloro-4-methylbiphenyl-3-yl)-4-hydroxy-7-(2-methoxyethyl)-1-oxaspiro[4.5]dec-3-en-2-one,
3-(4,4′-dichlorobiphenyl-3-yl)-4-hydroxy-7-(2-methoxyethyl)-1-oxaspiro[4.5]dec-3-en-2-one,
3-(4′-chloro-4-methylbiphenyl-3-yl)-4-hydroxy-8-(methoxymethyl)-1-oxaspiro[4.5]dec-3-en-2-one,
3-(4′-fluoro-4-methylbiphenyl-3-yl)-4-hydroxy-1-oxaspiro[4.5]dec-3-en-2-one,
3-(3′,4′-difluoro-4-methylbiphenyl-3-yl)-4-hydroxy-1-oxaspiro[4.4]non-3-en-2-one,
3-(3′-chloro-4′-fluoro-4-methylbiphenyl-3-yl)-4-hydroxy-1-oxaspiro[4.4]non-3-en-2-one,
3-(4-chloro-3′,4′,5′-trifluorobiphenyl-3-yl)-4-hydroxy-1-oxaspiro[4.4]non-3-en-2-one,
3-(4-chloro-3′,4′-difluorobiphenyl-3-yl)-4-hydroxy-1-oxaspiro[4.5]dec-3-en-2-one,
(5s,8r)-3-(4′-chloro-2,4-dimethylbiphenyl-3-yl)-4-hydroxy-1,9-dioxadispiro[4.2.4.2]tetradec-3-en-2-one,
3-(4,4′-dichlorobiphenyl-3-yl)-4-hydroxy-1,9-dioxadispiro[4.2.4.2]tetradec-3-en-2-one,
(5r,8s)-3-(4′-fluoro-4-methylbiphenyl-3-yl)-4-hydroxy-1,9-dioxadispiro[4.2.4.2]tetradec-3-en-2-one,
3-(4′-chloro-4-methylbiphenyl-3-yl)-4-hydroxy-1,9-dioxadispiro[4.2.4.2]tetradec-3-en-2-one,
(5r,8s)-3-(4′-chloro-4,6-dimethylbiphenyl-3-yl)-4-hydroxy-1,9-dioxadispiro[4.2.4.2]tetradec-3-en-2-one,
(5r,8s)-3-(4′-chloro-2,4,6-trimethylbiphenyl-3-yl)-4-hydroxy-1,9-dioxadispiro[4.2.4.2]tetradec-3-en-2-one,
3-(4′-chloro-2,4-dimethylbiphenyl-3-yl)-4-hydroxy-8-(2,2,2-trifluoroethoxy)-1-oxaspiro[4.5]dec-3-en-2-one,
3-(4′-chloro-4-methylbiphenyl-3-yl)-4-hydroxy-8-(2,2,2-trifluoroethoxy)-1-oxaspiro[4.5]dec-3-en-2-one,
2-(4′-fluoro-2,4-dimethylbiphenyl-3-yl)tetrahydro-1H-pyrazolo[1,2-a]pyridazin-1,3(2H)-dione
2-(4,4′-dichlorobiphenyl-3-yl)tetrahydro-1H-pyrazolo[1,2-a]pyridazine-1,3(2H)-dione
2-(4′-chloro-4-methylbiphenyl-3-yl)tetrahydro-1H-pyrazolo[1,2-a]pyridazine-1,3(2H)-dione
2-(3′,4-dichloro-4′-fluorobiphenyl-3-yl)tetrahydro-1H-pyrazolo[1,2-a]pyridazine-1,3(2H)-dione
8-(2′,4′-difluoro-4-methylbiphenyl-3-yl)tetrahydro-7H-pyrazolo[1,2-d][1,4,5]oxadiazepine-7,9(8H)-dione
8-(4′-chloro-4-methylbiphenyl-3-yl)tetrahydro-7H-pyrazolo[1,2-d][1,4,5]oxadiazepine-7,9(8H)-dione
8-(4′-fluoro-2,4-dimethylbiphenyl-3-yl)tetrahydro-7H-pyrazolo[1,2-d][1,4,5]oxadiazepine-7,9(8H)-dione
8-(4′-chloro-2,4-dimethylbiphenyl-3-yl)tetrahydro-7H-pyrazolo[1,2-d][1,4,5]oxadiazepine-7,9(8H)-dione
8-(2′,4,4′-trichlorobiphenyl-3-yl)tetrahydro-7H-pyrazolo[1,2-d][1,4,5]oxadiazepine-7,9(8H)-dione
8-(3′,4,4′-trichlorobiphenyl-3-yl)tetrahydro-7H-pyrazolo[1,2-d][1,4,5]oxadiazepine-7,9(8H)-dione
8-(4-chloro-2′,4′-difluorobiphenyl-3-yl)tetrahydro-7H-pyrazolo[1,2-d][1,4,5]oxadiazepine-7,9(8H)-dione
2-(4′-chloro-4-methylbiphenyl-3-yl)-6-fluoro-6-methyldihydro-1H,5H-pyrazolo[1,2-a]pyrazole-1,3(2H)-dione
2-(4,4′-dichlorobiphenyl-3-yl)-6-fluoro-6-methyldihydro-1H,5H-pyrazolo[1,2-a]pyrazole-1,3(2H)-dione
4-(4′-chloro-4-methylbiphenyl-3-yl)-1,2-dimethyl-1H-pyrazole-3,5(2H,4H)-dione
4-(4,4′-dichlorobiphenyl-3-yl)-1,2-dimethyl-1H-pyrazole-3,5(2H,4H)-dione
2-(4′-chloro-4-methylbiphenyl-3-yl)tetrahydro-1H-5,8-methanopyrazolo[1,2-a]pyridazine-1,3(2H)-dione
2-(4,4′-dichlorobiphenyl-3-yl)tetrahydro-1H-5,8-methanopyrazolo[1,2-a]pyridazine-1,3(2H)-dione
for preparing a medicament for the prophylaxis and therapy of diabetes, obesity, hyperlipidaemia, fatty livers and of disorders of the cardiovascular system.

2. A method for prophylaxis and therapy of diabetes, obesity, hyperlipidaemia, fatty livers and of disorders of the cardiovascular system which comprises administering one or more of the following compounds:
(5s,8s)-3-(4,4′-dichlorobiphenyl-3-yl)-4-hydroxy-8-(2,2,2-trifluoroethoxy)-1-azaspiro[4.5]dec-3-en-2-one,
(5s,8s)-3-(4′-chloro-2,4-dimethylbiphenyl-3-yl)-4-hydroxy-8-(2,2,2-trifluoroethoxy)-1-azaspiro[4.5]dec-3-en-2-one,
3-(4′-fluoro-2,4-dimethylbiphenyl-3-yl)-4-hydroxy-8-(2,2,2-trifluoroethoxy)-1-azaspiro[4.5]dec-3-en-2-one,
3-(4′-fluoro-4-methylbiphenyl-3-yl)-4-hydroxy-8-(2,2,2-trifluoroethoxy)-1-azaspiro[4.5]dec-3-en-2-one,
3-(4′-chloro-4-methylbiphenyl-3-yl)-4-hydroxy-8-(2,2,2-trifluoroethoxy)-1-azaspiro[4.5]dec-3-en-2-one,
11-(4′-chloro-2,4-dimethylbiphenyl-3-yl)-12-hydroxy-1-oxa-9-azadispiro[4.2.4.2]tetradec-11-en-10-one,
11-(3′,4′-difluoro-4-methylbiphenyl-3-yl)-12-hydroxy-1-oxa-9-azadispiro[4.2.4.2]tetradec-11-en-10-one,
4-hydroxy-3-(3′,4′,5′-trifluoro-4-methylbiphenyl-3-yl)-8-oxa-1-azaspiro[4.5]dec-3-en-2-one,
3-(3′,4′-difluoro-4-methylbiphenyl-3-yl)-4-hydroxy-8-oxa-1-azaspiro[4.5]dec-3-en-2-one,
3-(4-chloro-3′,4′,5′-trifluorobiphenyl-3-yl)-4-hydroxy-8-oxa-1-azaspiro[4.5]dec-3-en-2-one,
3-(4-chloro-3′-fluoro-4′-methylbiphenyl-3-yl)-4-hydroxy-8-oxa-1-azaspiro[4.5]dec-3-en-2-one,
3-(4′-fluoro-2,4-dimethylbiphenyl-3-yl)-4-hydroxy-1-azaspiro[4.5]dec-3-en-2-one,
3-(4-chloro-4′-fluorobiphenyl-3-yl)-4-hydroxy-8-oxa-1-azaspiro[4.5]dec-3-en-2-one,
(5s,8s)-3-(4′-fluoro-2,4,6-trimethylbiphenyl-3-yl)-4-hydroxy-8-methoxy-1-azaspiro[4.5]dec-3-en-2-one,
(5s,8s)-3-(3′-chloro-4′-fluoro-4-methylbiphenyl-3-yl)-4-hydroxy-8-methoxy-1-azaspiro[4.5]dec-3-en-2-one
(5s,8s)-3-(4-chloro-4′-fluorobiphenyl-3-yl)-4-hydroxy-8-methoxy-1-azaspiro[4.5]dec-3-en-2-one,
(5s,8s)-3-(4′-fluoro-2,4-dimethylbiphenyl-3-yl)-4-hydroxy-8-methoxy-1-azaspiro[4.5]dec-3-en-2-one,
3-(4′-chloro-4-methylbiphenyl-3-yl)-4-hydroxy-5-(methoxymethyl)-5-methyl-1,5-dihydro-2H-pyrrol-2-one,
3-(4′-chloro-4-methylbiphenyl-3-yl)-4-hydroxy-5-(2-methoxyethyl)-5-methyl-1,5-dihydro-2H-pyrrol-2-one,
3-(4′-chloro-2,4-dimethylbiphenyl-3-yl)-4-hydroxy-7-methoxy-1-azaspiro[4.4]non-3-en-2-one,
rel-(5R,7R)-3-(4′-chloro-4-methylbiphenyl-3-yl)-4-hydroxy-7-(2-methylpropoxy)-1-azaspiro[4.4]non-3-en-2-one,
rel-(5R,7S)-3-(4′-chloro-4-methylbiphenyl-3-yl)-7-(2-ethoxyethoxy)-4-hydroxy-1-azaspiro[4.4]non-3-en-2-one,
3-(4′-chloro-4-methylbiphenyl-3-yl)-4-hydroxy-7-(methoxymethyl)-1-azaspiro[4.5]dec-3-en-2-one,
3-(4,4′-dichlorobiphenyl-3-yl)-4-hydroxy-7-(methoxymethyl)-1-azaspiro[4.5]dec-3-en-2-one,
3-(4′-chloro-2,4-dimethylbiphenyl-3-yl)-4-hydroxy-8-(methoxymethyl)-1-azaspiro[4.5]dec-3-en-2-one,
3-(4,4′-dichlorobiphenyl-3-yl)-4-hydroxy-8-(methoxymethyl)-1-azaspiro[4.5]dec-3-en-2-one,
3-(4′-chloro-2,4-dimethylbiphenyl-3-yl)-4-hydroxy-7-(2-methoxyethyl)-1-azaspiro[4.5]dec-3-en-2-one,
3-(4′-chloro-4-methylbiphenyl-3-yl)-4-hydroxy-8-(methoxymethyl)-1-azaspiro[4.5]dec-3-en-2-one,
3-(4′-chloro-2,4-dimethylbiphenyl-3-yl)-4-hydroxy-8-(2-methoxyethyl)-1-azaspiro[4.5]dec-3-en-2-one,
3-(4,4′-dichlorobiphenyl-3-yl)-4-hydroxy-9,13-dioxa-1-azadispiro[4.2.5.2]pentadec-3-en-2-one,
3-(4′-chloro-4-methylbiphenyl-3-yl)-4-hydroxy-9,13-dioxa-1-azadispiro[4.2.5.2]pentadec-3-en-2-one,
11-(4,4′-dichlorobiphenyl-3-yl)-12-hydroxy-1,4-dioxa-9-azadispiro[4.2.4.2]tetradec-11-en-10-one,
11-(4′-chloro-2,4-dimethylbiphenyl-3-yl)-12-hydroxy-2-methyl-1,4-dioxa-9-azadispiro[4.2.4.2]tetradec-11-en-10-one,
11-(4′-chloro-4-methylbiphenyl-3-yl)-12-hydroxy-2-methyl-1,4-dioxa-9-azadispiro[4.2.4.2]tetradec-11-en-10-one,
11-(4′-chloro-4-methylbiphenyl-3-yl)-12-hydroxy-2,3-dimethyl-1,4-dioxa-9-azadispiro[4.2.4.2]tetradec-11-en-10-one,
3-(4,4′-dichlorobiphenyl-3-yl)-4-hydroxy-11,11-dimethyl-9,13-dioxa-1-azadispiro[4.2.5.2]pentadec-3-en-2-one,
3-(4′-chloro-4-methylbiphenyl-3-yl)-4-hydroxy-1′-methyl-9,13-dioxa-1-azadispiro[4.2.5.2]pentadec-3-en-2-one,
3-(4′-chloro-4-methylbiphenyl-3-yl)-4-hydroxy-11,11-dimethyl-9,13-dioxa-1-azadispiro[4.2.5.2]pentadec-3-en-2-one,
11-(4,4′-dichlorobiphenyl-3-yl)-12-hydroxy-2-methyl-1,4-dioxa-9-azadispiro[4.2.4.2]tetradec-1′-en-10-one,
3-(4,4′-dichlorobiphenyl-3-yl)-4-hydroxy-1′-methyl-9,13-dioxa-1-azadispiro[4.2.5.2]pentadec-3-en-2-one,
11-(4′-chloro-2,4-dimethylbiphenyl-3-yl)-12-hydroxy-1,4-dioxa-9-azadispiro[4.2.4.2]tetradec-1′-en-10-one,
11-(4,4′-dichlorobiphenyl-3-yl)-12-hydroxy-2,3-dimethyl-1,4-dioxa-9-azadispiro[4.2.4.2]tetradec-11-en-10-one,
3-(4′-chloro-2,4-dimethylbiphenyl-3-yl)-4-hydroxy-7-methoxy-1-azaspiro[4.5]dec-3-en-2-one,
3-(4′-chloro-2,4-dimethylbiphenyl-3-yl)-7-ethoxy-4-hydroxy-1-azaspiro[4.5]dec-3-en-2-one,
rel-(5R,7R)-3-(4,4′-dichlorobiphenyl-3-yl)-4-hydroxy-7-(2-methoxyethoxy)-1-azaspiro[4.5]dec-3-en-2-one,
3-(4′-chloro-2,4,6-trimethylbiphenyl-3-yl)-8-ethoxy-4-hydroxy-1-azaspiro[4.5]dec-3-en-2-one,
(5r,8r)-3-(4′-chloro-2,4-dimethylbiphenyl-3-yl)-4-hydroxy-8-methoxy-8-methyl-1-azaspiro[4.5]dec-3-en-2-one,
(5r,8r)-3-(4′-chloro-2,4-dimethylbiphenyl-3-yl)-8-ethyl-4-hydroxy-8-methoxy-1-azaspiro[4.5]dec-3-en-2-one,
(5r,8r)-8-ethyl-3-(4′-fluoro-2,4-dimethylbiphenyl-3-yl)-4-hydroxy-8-methoxy-1-azaspiro[4.5]dec-3-en-2-one,
3-(4′-chloro-4,6-dimethylbiphenyl-3-yl)-4-hydroxy-7-methoxy-1-oxaspiro[4.5]dec-3-en-2-one,
3-(4′-chloro-2,4,6-trimethylbiphenyl-3-yl)-4-hydroxy-7-methoxy-1-oxaspiro[4.5]dec-3-en-2-one,
3-(4,4′-dichlorobiphenyl-3-yl)-4-hydroxy-7-methoxy-1-oxaspiro[4.5]dec-3-en-2-one,
3-(4′-chloro-4-methylbiphenyl-3-yl)-4-hydroxy-7-methoxy-1-oxaspiro[4.5]dec-3-en-2-one,
11-(4′-chloro-4-methylbiphenyl-3-yl)-12-hydroxy-1,4,9-trioxadispiro[4.2.4.2]tetradec-11-en-10-one,
3-(4,4′-dichlorobiphenyl-3-yl)-4-hydroxy-8-(methoxymethyl)-1-oxaspiro[4.5]dec-3-en-2-one,
3-(4′-chloro-2,4-dimethylbiphenyl-3-yl)-4-hydroxy-8-(methoxymethyl)-1-oxaspiro[4.5]dec-3-en-2-one,
3-(4′-chloro-4-methylbiphenyl-3-yl)-4-hydroxy-7-(2-methoxyethyl)-1-oxaspiro[4.5]dec-3-en-2-one,
3-(4,4′-dichlorobiphenyl-3-yl)-4-hydroxy-7-(2-methoxyethyl)-1-oxaspiro[4.5]dec-3-en-2-one,
3-(4′-chloro-4-methylbiphenyl-3-yl)-4-hydroxy-8-(methoxymethyl)-1-oxaspiro[4.5]dec-3-en-2-one,
3-(4′-fluoro-4-methylbiphenyl-3-yl)-4-hydroxy-1-oxaspiro[4.5]dec-3-en-2-one,
3-(3′,4′-difluoro-4-methylbiphenyl-3-yl)-4-hydroxy-1-oxaspiro[4.4]non-3-en-2-one,
3-(3′-chloro-4′-fluoro-4-methylbiphenyl-3-yl)-4-hydroxy-1-oxaspiro[4.4]non-3-en-2-one,
3-(4-chloro-3′,4′,5′-trifluorobiphenyl-3-yl)-4-hydroxy-1-oxaspiro[4.4]non-3-en-2-one,
3-(4-chloro-3′,4′-difluorobiphenyl-3-yl)-4-hydroxy-1-oxaspiro[4.5]dec-3-en-2-one,
(5s,8r)-3-(4′-chloro-2,4-dimethylbiphenyl-3-yl)-4-hydroxy-1,9-dioxadispiro[4.2.4.2]tetradec-3-en-2-one,
3-(4,4′-dichlorobiphenyl-3-yl)-4-hydroxy-1,9-dioxadispiro[4.2.4.2]tetradec-3-en-2-one,
(5r,8s)-3-(4′-fluoro-4-methylbiphenyl-3-yl)-4-hydroxy-1,9-dioxadispiro[4.2.4.2]tetradec-3-en-2-one,
3-(4′-chloro-4-methylbiphenyl-3-yl)-4-hydroxy-1,9-dioxadispiro[4.2.4.2]tetradec-3-en-2-one,
(5r,8s)-3-(4′-chloro-4,6-dimethylbiphenyl-3-yl)-4-hydroxy-1,9-dioxadispiro[4.2.4.2]tetradec-3-en-2-one,
(5r,8s)-3-(4′-chloro-2,4,6-trimethylbiphenyl-3-yl)-4-hydroxy-1,9-dioxadispiro[4.2.4.2]tetradec-3-en-2-one,
3-(4′-chloro-2,4-dimethylbiphenyl-3-yl)-4-hydroxy-8-(2,2,2-trifluoroethoxy)-1-oxaspiro[4.5]dec-3-en-2-one,
3-(4′-chloro-4-methylbiphenyl-3-yl)-4-hydroxy-8-(2,2,2-trifluoroethoxy)-1-oxaspiro[4.5]dec-3-en-2-one,
2-(4′-fluoro-2,4-dimethylbiphenyl-3-yl)tetrahydro-1H-pyrazolo[1,2-a]pyridazin-1,3(2H)-dione
2-(4,4′-dichlorobiphenyl-3-yl)tetrahydro-1H-pyrazolo[1,2-a]pyridazine-1,3(2H)-dione
2-(4′-chloro-4-methylbiphenyl-3-yl)tetrahydro-1H-pyrazolo[1,2-a]pyridazine-1,3(2H)-dione
2-(3′,4-dichloro-4′-fluorobiphenyl-3-yl)tetrahydro-1H-pyrazolo[1,2-a]pyridazine-1,3(2H)-dione
8-(2′,4′-difluoro-4-methylbiphenyl-3-yl)tetrahydro-7H-pyrazolo[1,2-d][1,4,5]oxadiazepine-7,9(8H)-dione
8-(4′-chloro-4-methylbiphenyl-3-yl)tetrahydro-7H-pyrazolo[1,2-d][1,4,5]oxadiazepine-7,9(8H)-dione
8-(4′-fluoro-2,4-dimethylbiphenyl-3-yl)tetrahydro-7H-pyrazolo[1,2-d][1,4,5]oxadiazepine-7,9(8H)-dione
8-(4′-chloro-2,4-dimethylbiphenyl-3-yl)tetrahydro-7H-pyrazolo[1,2-d][1,4,5]oxadiazepine-7,9(8H)-dione
8-(2′,4,4′-trichlorobiphenyl-3-yl)tetrahydro-7H-pyrazolo[1,2-d][1,4,5]oxadiazepine-7,9(8H)-dione
8-(3′,4,4′-trichlorobiphenyl-3-yl)tetrahydro-7H-pyrazolo[1,2-d][1,4,5]oxadiazepine-7,9(8H)-dione
8-(4-chloro-2′,4′-difluorobiphenyl-3-yl)tetrahydro-7H-pyrazolo[1,2-d][1,4,5]oxadiazepine-7,9(8H)-dione
2-(4′-chloro-4-methylbiphenyl-3-yl)-6-fluoro-6-methyldihydro-1H,5H-pyrazolo[1,2-a]pyrazole-1,3(2H)-dione
2-(4,4′-dichlorobiphenyl-3-yl)-6-fluoro-6-methyldihydro-1H,5H-pyrazolo[1,2-a]pyrazole-1,3(2H)-dione
4-(4′-chloro-4-methylbiphenyl-3-yl)-1,2-dimethyl-1H-pyrazole-3,5(2H,4H)-dione
4-(4,4′-dichlorobiphenyl-3-yl)-1,2-dimethyl-1H-pyrazole-3,5(2H,4H)-dione
2-(4′-chloro-4-methylbiphenyl-3-yl)tetrahydro-1H-5,8-methanopyrazolo[1,2-a]pyridazine-1,3(2H)-dione
2-(4,4′-dichlorobiphenyl-3-yl)tetrahydro-1H-5,8-methanopyrazolo[1,2-a]pyridazine-1,3(2H)-dione
